SEQUENCE LISTING (271 sequences)

SEQ ID NO: 1 - protein, 9 aa, synthetic construct
YTFTSYWIH

SEQ ID NO: 2 - protein, 17 aa, synthetic construct
LIDPSDSYTNYNQKFRG

SEQ ID NO: 3 - protein, 8 aa, synthetic construct
ASSDPTGG

SEQ ID NO: 4 - protein, 16 aa, synthetic construct
RSSKSLLHSSGITYLS

SEQ ID NO: 5 - protein, 7 aa, synthetic construct
QMSNLAS

SEQ ID NO: 6 - protein, 9 aa, synthetic construct
AQNLELPLT

SEQ ID NO: 7 - protein, 115 aa, synthetic construct
QVQLVQSGAEVKKPGASVKVSCKASGYTFTSYWIHWVRQAPGQGLEWMGLIDPSDSYTNYNQKFRGRVTMTVDTSTSTAYMELSSLRSEDTAVYYCASSDPTGGWGQGTTVTVSS

SEQ ID NO: 8 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 107 aa, synthetic construct [sequence omitted]

SEQ ID NO: 10 - protein, 112 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQNLELPLTFGGGTKVEIK

SEQ ID NO: 11 - protein, 444 aa, synthetic construct
QVQLVQSGAEVKKPGASVKVSCKASGYTFTSYWIHWVRQAPGQGLEWMGLIDPSDSYTNYNQKFRGRVTMTVDTSTSTAYMELSSLRSEDTAVYYCASSDPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 135 aa, synthetic construct
AEDVGVYYCAQNLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 9 aa, synthetic construct
YTFTSSWIH

SEQ ID NO: 14 - protein, 17 aa, synthetic construct
LIDPSDSYTNYNQKFRG

SEQ ID NO: 15 - protein, 8 aa, synthetic construct
ASSDPHGG

SEQ ID NO: 16 - protein, 16 aa, synthetic construct
RSSKSLLHSSGITMLS

SEQ ID NO: 17 - protein, 7 aa, synthetic construct
QMSNLAS

SEQ ID NO: 18 - protein, 9 aa, synthetic construct
AQSLELPLT

SEQ ID NO: 19 - protein, 115 aa, synthetic construct
QVQLVQSGAEVKKPGASVKVSCKASGYTFTSSWIHWVRQAPGQGLEWMGLIDPSDSYTNYNQKFRGRVTMTVDTSTSTAYMELSSLRSEDTAVYYCASSDPHGGWGQGTTVTVSS

SEQ ID NO: 20 - protein, 329 aa, synthetic construct; identical to SEQ ID NO: 8

SEQ ID NO: 21 - protein, 107 aa, synthetic construct [sequence omitted]

SEQ ID NO: 22 - protein, 112 aa, synthetic construct
DIVMTQSPLSLPVTPGEPASISCRSSKSLLHSSGITMLSWYLQKPGQSPQLLIYQMSNLASGVPDRFSGSGSGTDFTLKISRVEAEDVGVYYCAQSLELPLTFGGGTKVEIK

SEQ ID NO: 23 - protein, 444 aa, synthetic construct
QVQLVQSGAEVKKPGASVKVSCKASGYTFTSSWIHWVRQAPGQGLEWMGLIDPSDSYTNYNQKFRGRVTMTVDTSTSTAYMELSSLRSEDTAVYYCASSDPHGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 219 aa, synthetic construct
DIVMTQSPLSLPVTPGEPASISCRSSKSLLHSSGITMLSWYLQKPGQSPQLLIYQMSNLASGVPDRFSGSGSGTDFTLKISRVEAEDVGVYYCAQSLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 9 aa, synthetic construct
YTFTSSWIH

SEQ ID NO: 26 - protein, 17 aa, synthetic construct
LIDPSDSYTNYNQKFRG

SEQ ID NO: 27 - protein, 8 aa, synthetic construct
ASSAPTGG

SEQ ID NO: 28 - protein, 16 aa, synthetic construct
RSSKSLLHSSGITYLS

SEQ ID NO: 29 - protein, 7 aa, synthetic construct
QMSNLAS

SEQ ID NO: 30 - protein, 9 aa, synthetic construct
AQALELPLT

SEQ ID NO: 31 - protein, 115 aa, synthetic construct
QVQLVQSGAEVKKPGASVKVSCKASGYTFTSSWIHWVRQAPGQGLEWMGLIDPSDSYTNYNQKFRGRVTMTVDTSTSTAYMELSSLRSEDTAVYYCASSAPTGGWGQGTTVTVSS

SEQ ID NO: 32 - protein, 329 aa, synthetic construct; identical to SEQ ID NO: 8

SEQ ID NO: 33 - protein, 107 aa, synthetic construct [sequence omitted]

SEQ ID NO: 34 - protein, 112 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQALELPLTFGGGTKVEIK

SEQ ID NO: 35 - protein, 444 aa, synthetic construct
QVQLVQSGAEVKKPGASVKVSCKASGYTFTSSWIHWVRQAPGQGLEWMGLIDPSDSYTNYNQKFRGRVTMTVDTSTSTAYMELSSLRSEDTAVYYCASSAPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 219 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQALELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 9 aa, synthetic construct
YTFTSYWIH

SEQ ID NO: 38 - protein, 17 aa, synthetic construct
LIDPSDSYTNYNQKFRG

SEQ ID NO: 39 - protein, 8 aa, synthetic construct
ASSDATGG

SEQ ID NO: 40 - protein, 16 aa, synthetic construct
RSSKSLLHSSGITYLS

SEQ ID NO: 41 - protein, 7 aa, synthetic construct
QMSNLAS

SEQ ID NO: 42 - protein, 9 aa, synthetic construct
AQALELPLT

SEQ ID NO: 43 - protein, 115 aa, synthetic construct
QVQLVQSGAEVKKPGASVKVSCKASGYTFTSYWIHWVRQAPGQGLEWMGLIDPSDSYTNYNQKFRGRVTMTVDTSTSTAYMELSSLRSEDTAVYYCASSDATGGWGQGTTVTVSS

SEQ ID NO: 44 - protein, 329 aa, synthetic construct; identical to SEQ ID NO: 8

SEQ ID NO: 45 - protein, 107 aa, synthetic construct [sequence omitted]

SEQ ID NO: 46 - protein, 112 aa, synthetic construct; identical to SEQ ID NO: 34

SEQ ID NO: 47 - protein, 444 aa, synthetic construct
QVQLVQSGAEVKKPGASVKVSCKASGYTFTSYWIHWVRQAPGQGLEWMGLIDPSDSYTNYNQKFRGRVTMTVDTSTSTAYMELSSLRSEDTAVYYCASSDATGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 219 aa, synthetic construct; identical to SEQ ID NO: 36

SEQ ID NO: 49 - protein, 9 aa, synthetic construct
YTFTSSWIH

SEQ ID NO: 50 - protein, 17 aa, synthetic construct
LIDPSDSYTNYNQKFRG

SEQ ID NO: 51 - protein, 8 aa, synthetic construct
ASSDPHGG

SEQ ID NO: 52 - protein, 16 aa, synthetic construct
RSSKSLLHSSGITYLS

SEQ ID NO: 53 - protein, 7 aa, synthetic construct
QMSNLAS

SEQ ID NO: 54 - protein, 9 aa, synthetic construct
AQSLELPLT

SEQ ID NO: 55 - protein, 115 aa, synthetic construct; identical to SEQ ID NO: 19

SEQ ID NO: 56 - protein, 329 aa, synthetic construct; identical to SEQ ID NO: 8

SEQ ID NO: 57 - protein, 107 aa, synthetic construct [sequence omitted]

SEQ ID NO: 58 - protein, 112 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQSLELPLTFGGGTKVEIK

SEQ ID NO: 59 - protein, 444 aa, synthetic construct; identical to SEQ ID NO: 23

SEQ ID NO: 60 - protein, 219 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQSLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 9 aa, synthetic construct
YTFTSTWIH

SEQ ID NO: 62 - protein, 17 aa, synthetic construct
LIDPSDSYTNYNQKFRG

SEQ ID NO: 63 - protein, 8 aa, synthetic construct
ASSKPTGG

SEQ ID NO: 64 - protein, 16 aa, synthetic construct
RSSKSLLHSSGITYIS

SEQ ID NO: 65 - protein, 7 aa, synthetic construct
QMSNLAS

SEQ ID NO: 66 - protein, 9 aa, synthetic construct
AQNLELPLT

SEQ ID NO: 67 - protein, 115 aa, synthetic construct
QVQLVQSGAEVKKPGASVKVSCKASGYTFTSTWIHWVRQAPGQGLEWMGLIDPSDSYTNYNQKFRGRVTMTVDTSTSTAYMELSSLRSEDTAVYYCASSKPTGGWGQGTTVTVSS

SEQ ID NO: 68 - protein, 329 aa, synthetic construct; identical to SEQ ID NO: 8

SEQ ID NO: 69 - protein, 107 aa, synthetic construct [sequence omitted]

SEQ ID NO: 70 - protein, 112 aa, synthetic construct
DIVMTQSPLSLPVTPGEPASISCRSSKSLLHSSGITYISWYLQKPGQSPQLLIYQMSNLASGVPDRFSGSGSGTDFTLKISRVEAEDVGVYYCAQNLELPLTFGGGTKVEIK

SEQ ID NO: 71 - protein, 444 aa, synthetic construct
QVQLVQSGAEVKKPGASVKVSCKASGYTFTSTWIHWVRQAPGQGLEWMGLIDPSDSYTNYNQKFRGRVTMTVDTSTSTAYMELSSLRSEDTAVYYCASSKPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 219 aa, synthetic construct
DIVMTQSPLSLPVTPGEPASISCRSSKSLLHSSGITYISWYLQKPGQSPQLLIYQMSNLASGVPDRFSGSGSGTDFTLKISRVEAEDVGVYYCAQNLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 9 aa, synthetic construct
YTFTSTWIH

SEQ ID NO: 74 - protein, 17 aa, synthetic construct
LIDPSDSYTNYNQKFRG

SEQ ID NO: 75 - protein, 8 aa, synthetic construct
ASSKPTGG

SEQ ID NO: 76 - protein, 16 aa, synthetic construct
RSSKSLLHSSGITYLS

SEQ ID NO: 77 - protein, 7 aa, synthetic construct
QMSNLAS

SEQ ID NO: 78 - protein, 9 aa, synthetic construct
AQNLELPLT

SEQ ID NO: 79 - protein, 115 aa, synthetic construct; identical to SEQ ID NO: 67

SEQ ID NO: 80 - protein, 329 aa, synthetic construct; identical to SEQ ID NO: 8

SEQ ID NO: 81 - protein, 107 aa, synthetic construct [sequence omitted]

SEQ ID NO: 82 - protein, 112 aa, synthetic construct; identical to SEQ ID NO: 10

SEQ ID NO: 83 - protein, 444 aa, synthetic construct; identical to SEQ ID NO: 71

SEQ ID NO: 84 - protein, 219 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQNLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 9 aa, synthetic construct
YTFTSAWIH

SEQ ID NO: 86 - protein, 17 aa, synthetic construct
LIDPSDSYTNYNQKFRG

SEQ ID NO: 87 - protein, 8 aa, synthetic construct
ASSDPYGG

SEQ ID NO: 88 - protein, 16 aa, synthetic construct
RSSKSLLHSSGITYLS

SEQ ID NO: 89 - protein, 7 aa, synthetic construct
QMSNKAS

SEQ ID NO: 90 - protein, 9 aa, synthetic construct
AQSLELPLT

SEQ ID NO: 91 - protein, 115 aa, synthetic construct
QVQLVQSGAEVKKPGASVKVSCKASGYTFTSAWIHWVRQAPGQGLEWMGLIDPSDSYTNYNQKFRGRVTMTVDTSTSTAYMELSSLRSEDTAVYYCASSDPYGGWGQGTTVTVSS

SEQ ID NO: 92 - protein, 329 aa, synthetic construct; identical to SEQ ID NO: 8

SEQ ID NO: 93 - protein, 107 aa, synthetic construct [sequence omitted]

SEQ ID NO: 94 - protein, 112 aa, synthetic construct
DIVMTQSPLSLPVTPGEPASISCRSSKSLLHSSGITYLSWYLQKPGQSPQLLIYQMSNKASGVPDRFSGSGSGTDFTLKISRVEAEDVGVYYCAQSLELPLTFGGGTKVEIK

SEQ ID NO: 95 - protein, 444 aa, synthetic construct
QVQLVQSGAEVKKPGASVKVSCKASGYTFTSAWIHWVRQAPGQGLEWMGLIDPSDSYTNYNQKFRGRVTMTVDTSTSTAYMELSSLRSEDTAVYYCASSDPYGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 556 aa, synthetic construct
DIVMTQSPLSLPVTPGEPASISCRSSKSLLHSSGITYLSWYLQKPGQSPQLLIYQMSNKASGVPDRFSGSGSGTDFTLKISRVEAEDVGVYYCAQSLELPLTFGGGTKVEIKQVQLVQSGAEVKKPGASVKVSCKASGYTFTSAWIHWVRQAPGQGLEWMGLIDPSDSYTNYNQKFRGRVTMTVDTSTSTAYMELSSLRSEDTAVYYCASSDPYGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 9 aa, synthetic construct
YTFTSTWIH

SEQ ID NO: 98 - protein, 17 aa, synthetic construct
LIDPSDSYTNYNQKFRG

SEQ ID NO: 99 - protein, 8 aa, synthetic construct
ASSDLTGG

SEQ ID NO: 100 - protein, 16 aa, synthetic construct
RSSKSLLHSSGITYLS

SEQ ID NO: 101 - protein, 7 aa, synthetic construct
QMSNLAS

SEQ ID NO: 102 - protein, 9 aa, synthetic construct
AQALELPLT

SEQ ID NO: 103 - protein, 115 aa, synthetic construct
QVQLVQSGAEVKKPGASVKVSCKASGYTFTSTWIHWVRQAPGQGLEWMGLIDPSDSYTNYNQKFRGRVTMTVDTSTSTAYMELSSLRSEDTAVYYCASSDLTGGWGQGTTVTVSS

SEQ ID NO: 104 - protein, 329 aa, synthetic construct; identical to SEQ ID NO: 8

SEQ ID NO: 105 - protein, 107 aa, synthetic construct [sequence omitted]

SEQ ID NO: 106 - protein, 112 aa, synthetic construct
DIVMTQSPLSLPVTPGEPASISCRSSKSLLHSSGITYLSWYLQKPGQSPQLLIYQMSNLASGVPDRFSGSGSGTDFSLKISRVEAEDVGVYYCAQALELPLTFGGGTKVEIK

SEQ ID NO: 107 - protein, 444 aa, synthetic construct
QVQLVQSGAEVKKPGASVKVSCKASGYTFTSTWIHWVRQAPGQGLEWMGLIDPSDSYTNYNQKFRGRVTMTVDTSTSTAYMELSSLRSEDTAVYYCASSDLTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 219 aa, synthetic construct
DIVMTQSPLSLPVTPGEPASISCRSSKSLLHSSGITYLSWYLQKPGQSPQLLIYQMSNLASGVPDRFSGSGSGTDFSLKISRVEAEDVGVYYCAQALELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 9 aa, synthetic construct
YTFKSYWIH

SEQ ID NO: 110 - protein, 17 aa, synthetic construct
LIDPSDSYTNYNQIFRG

SEQ ID NO: 111 - protein, 8 aa, synthetic construct
ASSDATGG

SEQ ID NO: 112 - protein, 16 aa, synthetic construct
RSSKSLLHSSGITYLS

SEQ ID NO: 113 - protein, 7 aa, synthetic construct
QMSNLAS

SEQ ID NO: 114 - protein, 9 aa, synthetic construct
AQALELPLT

SEQ ID NO: 115 - protein, 115 aa, synthetic construct
QVQLVQSGAEVKKPGASVKVSCKASGYTFKSYWIHWVRQAPGQGLEWMGLIDPSDSYTNYNQIFRGRVTMTVDTSTSTAYMELSSLRSEDTAVYYCASSDATGGWGQGTTVTVSS

SEQ ID NO: 116 - protein, 329 aa, synthetic construct; identical to SEQ ID NO: 8

SEQ ID NO: 117 - protein, 107 aa, synthetic construct [sequence omitted]

SEQ ID NO: 118 - protein, 112 aa, synthetic construct; identical to SEQ ID NO: 34

SEQ ID NO: 119 - protein, 444 aa, synthetic construct
QVQLVQSGAEVKKPGASVKVSCKASGYTFKSYWIHWVRQAPGQGLEWMGLIDPSDSYTNYNQIFRGRVTMTVDTSTSTAYMELSSLRSEDTAVYYCASSDATGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 219 aa, synthetic construct; identical to SEQ ID NO: 36

SEQ ID NO: 121 - protein, 9 aa, synthetic construct
YTFTSTWIH

SEQ ID NO: 122 - protein, 17 aa, synthetic construct
LIDPSDSYTNYNQKFRG

SEQ ID NO: 123 - protein, 8 aa, synthetic construct
ASSKPTGG

SEQ ID NO: 124 - protein, 16 aa, synthetic construct
RSSKSLLHSSGITYLS

SEQ ID NO: 125 - protein, 7 aa, synthetic construct
QMSNLAS

SEQ ID NO: 126 - protein, 9 aa, synthetic construct
AQNLELPLT

SEQ ID NO: 127 - protein, 115 aa, synthetic construct
QVQLVQSGAEVKKPGASVKVSCKASGYTFTSYWIHWVRQAPGQGLEWIGLIDPSDSYTNYNQKFRGRATLTVDTSTSTAYMELSSLRSEDTAVYYCASSDPTGGWGQGTTVTVSS

SEQ ID NO: 128 - protein, 329 aa, synthetic construct; identical to SEQ ID NO: 8

SEQ ID NO: 129 - protein, 107 aa, synthetic construct [sequence omitted]

SEQ ID NO: 130 - protein, 112 aa, synthetic construct; identical to SEQ ID NO: 10

SEQ ID NO: 131 - protein, 444 aa, synthetic construct
QVQLVQSGAEVKKPGASVKVSCKASGYTFTSYWIHWVRQAPGQGLEWIGLIDPSDSYTNYNQKFRGRATLTVDTSTSTAYMELSSLRSEDTAVYYCASSDPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 219 aa, synthetic construct; identical to SEQ ID NO: 84

SEQ ID NO: 133 - protein, 9 aa, synthetic construct
YTFTSYWIH

SEQ ID NO: 134 - protein, 17 aa, synthetic construct
LIDPSDSYTNYNQKFRG

SEQ ID NO: 135 - protein, 8 aa, synthetic construct
ASSDATGG

SEQ ID NO: 136 - protein, 16 aa, synthetic construct
RSSKSLLHSSGHTYLS

SEQ ID NO: 137 - protein, 7 aa, synthetic construct
QMSNLAS

SEQ ID NO: 138 - protein, 9 aa, synthetic construct
AQALELPLT

SEQ ID NO: 139 - protein, 115 aa, synthetic construct
QVQLVQSGAEVKKPGASVKVSCKASGYTFTSYWIHWVRQAPGQGLEWIGLIDPSDSYTNYNQKFRGRATLTVDTSTSTAYMELSSLRSEDTAVYYCASSDATGGWGQGTTVTVSS

SEQ ID NO: 140 - protein, 329 aa, synthetic construct; identical to SEQ ID NO: 8

SEQ ID NO: 141 - protein, 107 aa, synthetic construct [sequence omitted]

SEQ ID NO: 142 - protein, 112 aa, synthetic construct
DIVMTQSPLSLPVTPGEPASISCRSSKSLLHSSGHTYLSWYLQKPGQPPQLLIYQMSNLASGVPDRFSGSGSGTDFTLKISRVEAEDVGVYYCAQALELPLTFGGGTKVEIK

SEQ ID NO: 143 - protein, 444 aa, synthetic construct
QVQLVQSGAEVKKPGASVKVSCKASGYTFTSYWIHWVRQAPGQGLEWIGLIDPSDSYTNYNQKFRGRATLTVDTSTSTAYMELSSLRSEDTAVYYCASSDATGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 219 aa, synthetic construct
DIVMTQSPLSLPVTPGEPASISCRSSKSLLHSSGHTYLSWYLQKPGQPPQLLIYQMSNLASGVPDRFSGSGSGTDFTLKISRVEAEDVGVYYCAQALELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 9 aa, synthetic construct
YTFTSVWIH

SEQ ID NO: 146 - protein, 17 aa, synthetic construct
LIDPSDSYTNYNQKFRG

SEQ ID NO: 147 - protein, 8 aa, synthetic construct
ASSRPTGG

SEQ ID NO: 148 - protein, 16 aa, synthetic construct
RSSKSLLHSSGITYLS

SEQ ID NO: 149 - protein, 7 aa, synthetic construct
QMSNLAS

SEQ ID NO: 150 - protein, 9 aa, synthetic construct
AQNLELPLT

SEQ ID NO: 151 - protein, 115 aa, synthetic construct
QVQLVQSGAEVKKPGASVKVSCKASGYTFTSVWIHWVRQAPGQGLEWIGLIDPSDSYTNYNQKFRGRATLTVDTSTSTAYMELSSLRSEDTAVYYCASSRPTGGWGQGTTVTVSS

SEQ ID NO: 152 - protein, 329 aa, synthetic construct; identical to SEQ ID NO: 8

SEQ ID NO: 153 - protein, 107 aa, synthetic construct [sequence omitted]

SEQ ID NO: 154 - protein, 112 aa, synthetic construct; identical to SEQ ID NO: 10

SEQ ID NO: 155 - protein, 444 aa, synthetic construct
QVQLVQSGAEVKKPGASVKVSCKASGYTFTSVWIHWVRQAPGQGLEWIGLIDPSDSYTNYNQKFRGRATLTVDTSTSTAYMELSSLRSEDTAVYYCASSRPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 219 aa, synthetic construct; identical to SEQ ID NO: 84

SEQ ID NO: 157 - protein, 9 aa, synthetic construct
YTFTSTWIH

SEQ ID NO: 158 - protein, 17 aa, synthetic construct
LIDPSDSYTNYNQKFRG

SEQ ID NO: 159 - protein, 8 aa, synthetic construct
ASSKPTGG

SEQ ID NO: 160 - protein, 16 aa, synthetic construct
RSSKSLLHSSGITYLS

SEQ ID NO: 161 - protein, 7 aa, synthetic construct
QMSNLAS

SEQ ID NO: 162 - protein, 9 aa, synthetic construct
AQNLELPLT

SEQ ID NO: 163 - protein, 115 aa, synthetic construct
QVQLVQSGAEVKKPGASVKVSCKASGYTFTSTWIHWVRQAPGQGLEWIGLIDPSDSYTNYNQKFRGRATLTVDTSTSTAYMELSSLRSEDTAVYYCASSKPTGGWGQGTTVTVSS

SEQ ID NO: 164 - protein, 329 aa, synthetic construct; identical to SEQ ID NO: 8

SEQ ID NO: 165 - protein, 107 aa, synthetic construct [sequence omitted]

SEQ ID NO: 166 - protein, 112 aa, synthetic construct; identical to SEQ ID NO: 10

SEQ ID NO: 167 - protein, 444 aa, synthetic construct
QVQLVQSGAEVKKPGASVKVSCKASGYTFTSTWIHWVRQAPGQGLEWIGLIDPSDSYTNYNQKFRGRATLTVDTSTSTAYMELSSLRSEDTAVYYCASSKPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 219 aa, synthetic construct; identical to SEQ ID NO: 84

SEQ ID NO: 169 - protein, 9 aa, synthetic construct
YTFTSTWIH

SEQ ID NO: 170 - protein, 17 aa, synthetic construct
LIDPSDSYTNYNQKFVG

SEQ ID NO: 171 - protein, 8 aa, synthetic construct
ASSLPTGG

SEQ ID NO: 172 - protein, 16 aa, synthetic construct
RSSKSLLHSSGITYLS

SEQ ID NO: 173 - protein, 7 aa, synthetic construct
QMSNLAS

SEQ ID NO: 174 - protein, 9 aa, synthetic construct
AQQLELPLT

SEQ ID NO: 175 - protein, 115 aa, synthetic construct
QVQLVQSGAEVKKPGASVKVSCKASGYTFTSTWIHWVRQAPGQGLEWIGLIDPSDSYTNYNQKFVGRATLTVDTSTSTAYMELSSLRSEDTAVYYCASSLPTGGWGQGTTVTVSS

SEQ ID NO: 176 - protein, 329 aa, synthetic construct; identical to SEQ ID NO: 8

SEQ ID NO: 177 - protein, 107 aa, synthetic construct [sequence omitted]

SEQ ID NO: 178 - protein, 112 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQQLELPLTFGGGTKVEIK

SEQ ID NO: 179 - protein, 444 aa, synthetic construct
QVQLVQSGAEVKKPGASVKVSCKASGYTFTSTWIHWVRQAPGQGLEWIGLIDPSDSYTNYNQKFVGRATLTVDTSTSTAYMELSSLRSEDTAVYYCASSLPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - protein, 219 aa, synthetic construct
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQQLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 9 aa, synthetic construct
YTFTSVWIH

SEQ ID NO: 182 - protein, 16 aa, synthetic construct
LIDPSDSYTNYNQKFR

SEQ ID NO: 183 - protein, 8 aa, synthetic construct
ASSQPTGG

SEQ ID NO: 184 - protein, 16 aa, synthetic construct
RSSKSLLHSSGITYLS

SEQ ID NO: 185 - protein, 7 aa, synthetic construct
QMSNLAS

SEQ ID NO: 186 - protein, 9 aa, synthetic construct
AQNLELPLT

SEQ ID NO: 187 - protein, 115 aa, synthetic construct
QVQLVQSGAEVKKPGASVKVSCKASGYTFTSVWIHWVRQAPGQGLEWIGLIDPSDSYTNYNQKFRGRATLTVDTSTSTAYMELSSLRSEDTAVYYCASSQPTGGWGQGTTVTVSS

SEQ ID NO: 188 - protein, 329 aa, synthetic construct; identical to SEQ ID NO: 8

SEQ ID NO: 189 - protein, 107 aa, synthetic construct [sequence omitted]

SEQ ID NO: 190 - protein, 112 aa, synthetic construct; identical to SEQ ID NO: 10

SEQ ID NO: 191 - protein, 444 aa, synthetic construct
QVQLVQSGAEVKKPGASVKVSCKASGYTFTSVWIHWVRQAPGQGLEWIGLIDPSDSYTNYNQKFRGRATLTVDTSTSTAYMELSSLRSEDTAVYYCASSQPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 219 aa, synthetic construct; identical to SEQ ID NO: 84

SEQ ID NO: 193 - protein, 9 aa, synthetic construct
YTFTSTWIH

SEQ ID NO: 194 - protein, 16 aa, synthetic construct
LIDPSDSYTNYNQKFR

SEQ ID NO: 195 - protein, 8 aa, synthetic construct
ASSDLTGG

SEQ ID NO: 196 - protein, 16 aa, synthetic construct
RSSKSLLHSSGITYLS

SEQ ID NO: 197 - protein, 7 aa, synthetic construct
QMSNLAS

SEQ ID NO: 198 - protein, 9 aa, synthetic construct
AQALELPLT

SEQ ID NO: 199 - protein, 115 aa, synthetic construct; identical to SEQ ID NO: 103

SEQ ID NO: 200 - protein, 329 aa, synthetic construct; identical to SEQ ID NO: 8

SEQ ID NO: 201 - protein, 107 aa, synthetic construct [sequence omitted]

SEQ ID NO: 202 - protein, 112 aa, synthetic construct; identical to SEQ ID NO: 106

SEQ ID NO: 203 - protein, 444 aa, synthetic construct; identical to SEQ ID NO: 107

SEQ ID NO: 204 - protein, 219 aa, synthetic construct; identical to SEQ ID NO: 108

SEQ ID NO: 205 - protein, 9 aa, synthetic construct
YRFTSYWIH

SEQ ID NO: 206 - protein, 17 aa, synthetic construct
LIDPSDSYTNYNQKFRG

SEQ ID NO: 207 - protein, 8 aa, synthetic construct
ASSDATGG

SEQ ID NO: 208 - protein, 16 aa, synthetic construct
VSSKSLLHSSGITYLS

SEQ ID NO: 209 - protein, 7 aa, synthetic construct
QMSNLGS

SEQ ID NO: 210 - protein, 9 aa, synthetic construct
AQALELPLT

SEQ ID NO: 211 - protein, 115 aa, synthetic construct
QVQLVQSGAEVKKPGASVKVSCKASGYRFTSYWIHWVRQAPGQGLEWIGLIDPSDSYTNYNQKFRGRATLTVDTSTSTAYMELSSLRSEDTAVYYCASSDATGGWGQGTTVTVSS

SEQ ID NO: 212 - protein, 329 aa, synthetic construct; identical to SEQ ID NO: 8

SEQ ID NO: 213 - protein, 107 aa, synthetic construct [sequence omitted]

SEQ ID NO: 214 - protein, 112 aa, synthetic construct
DIVMTQSPLSLPVTPGEPASISCVSSKSLLHSSGITYLSWYLQKPGQSPQLLIYQMSNLGSGVPDRFSGSGSGTDFTLKISRVEAEDVGVYYCAQALELPLTFGGGTKVEIK

SEQ ID NO: 215 - protein, 444 aa, synthetic construct
QVQLVQSGAEVKKPGASVKVSCKASGYRFTSYWIHWVRQAPGQGLEWIGLIDPSDSYTNYNQKFRGRATLTVDTSTSTAYMELSSLRSEDTAVYYCASSDATGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 216 - protein, 219 aa, synthetic construct
DIVMTQSPLSLPVTPGEPASISCVSSKSLLHSSGITYLSWYLQKPGQSPQLLIYQMSNLGSGVPDRFSGSGSGTDFTLKISRVEAEDVGVYYCAQALELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 9 aa, synthetic construct
YTHTSYWIH

SEQ ID NO: 218 - protein, 16 aa, synthetic construct
LIDPSDSYTNYNQKFR

SEQ ID NO: 219 - protein, 8 aa, synthetic construct
ASSRPTGG

SEQ ID NO: 220 - protein, 16 aa, synthetic construct
RSSKSLLHSSGITYLS

SEQ ID NO: 221 - protein, 7 aa, synthetic construct
QMSNLAS

SEQ ID NO: 222 - protein, 9 aa, synthetic construct
AQNLELPLT

SEQ ID NO: 223 - protein, 115 aa, synthetic construct
QVQLVQSGAEVKKPGASVKVSCKASGYTHTSYWIHWVRQAPGQGLEWIGLIDPSDSYTNYNQKFRGRATLTVDTSTSTAYMELSSLRSEDTAVYYCASSRPTGGWGQGTTVTVSS

SEQ ID NO: 224 - protein, 329 aa, synthetic construct; identical to SEQ ID NO: 8

SEQ ID NO: 225 - protein, 107 aa, synthetic construct [sequence omitted]

SEQ ID NO: 226 - protein, 112 aa, synthetic construct; identical to SEQ ID NO: 10

SEQ ID NO: 227 - protein, 444 aa, synthetic construct
QVQLVQSGAEVKKPGASVKVSCKASGYTHTSYWIHWVRQAPGQGLEWIGLIDPSDSYTNYNQKFRGRATLTVDTSTSTAYMELSSLRSEDTAVYYCASSRPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 219 aa, synthetic construct; identical to SEQ ID NO: 84

SEQ ID NO: 229 - protein, 9 aa, synthetic construct
YTFTEYWIH

SEQ ID NO: 230 - protein, 16 aa, synthetic construct
LIDPSDSYTNYNQRFR

SEQ ID NO: 231 - protein, 8 aa, synthetic construct
ASSDATGG

SEQ ID NO: 232 - protein, 16 aa, synthetic construct
HSSKSLLHSSGITYLS

SEQ ID NO: 233 - protein, 7 aa, synthetic construct
QMSNLAS

SEQ ID NO: 234 - protein, 9 aa, synthetic construct
AQSLELPLT

SEQ ID NO: 235 - protein, 115 aa, synthetic construct
QVQLVQSGAEVKKPGASVKASCKASGYTFTEYWIHWVRQAPGQGLEWIGLIDPSDSYTNYNQRFRGRATLTVDTSTSTAYMELSSLRSEDTAVYYCASSDATGGWGQGTTVTVSS

SEQ ID NO: 236 - protein, 329 aa, synthetic construct; identical to SEQ ID NO: 8

SEQ ID NO: 237 - protein, 107 aa, synthetic construct [sequence omitted]

SEQ ID NO: 238 - protein, 112 aa, synthetic construct
DIVMTQSPLSLPVTPGEPASISCHSSKSLLHSSGITYLSWYLQKPGQSPQLLIYQMSNLASGVPDRFSGSGSGTDFTLKISRVEAEDVGVYYCAQSLELPLTFGGGTKVEIK

SEQ ID NO: 239 - protein, 444 aa, synthetic construct
QVQLVQSGAEVKKPGASVKASCKASGYTFTEYWIHWVRQAPGQGLEWIGLIDPSDSYTNYNQRFRGRATLTVDTSTSTAYMELSSLRSEDTAVYYCASSDATGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 240 - protein, 219 aa, synthetic construct
DIVMTQSPLSLPVTPGEPASISCHSSKSLLHSSGITYLSWYLQKPGQSPQLLIYQMSNLASGVPDRFSGSGSGTDFTLKISRVEAEDVGVYYCAQSLELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 19 aa, Homo sapiens
YSMKKTTMKIIPFNRLTIG

SEQ ID NO: 242 - DNA, 717 bp, synthetic construct
  source 1..717: mol_type = other DNA
atgactaggctgaccgtgctggccctgcttgccggactcttggcctcctcgagagcggatattgtgatgactcagagcccactctccctgcccgtgactcctggggaacccgcctcgatcagctgtagatcgtccaagtcacttctccactcgtccgggatcacctacctgtcgtggtatttgcaaaagccaggacagagcccgcagctcctcatctaccaaatgagcaacctggcttccggtgtcccggatcggttctcggggtccggatctggcaccgacttcacgctgaaaatttcccgcgtggaagccgaggacgtgggagtgtactactgcgcacaaaacctggaactgcccctgaccttcggtggcggcactaaggtcgaaatcaagcggaccgtggcagctccgtccgtgttcatcttcccgccttccgacgagcagctgaagtccggaaccgcctccgtcgtgtgcctgctcaacaacttttaccctcgcgaggccaaggtccagtggaaggtcgataacgcgctgcagagcggaaatagccaggagagcgtgaccgagcaggactccaaggactcaacctactcactgagctccactctgaccctgtcaaaggcggactacgagaagcacaaagtgtacgcctgcgaagtgacacatcagggcctgtccagtcccgtgaccaagtccttcaaccggggcgaatgctag

SEQ ID NO: 243 - DNA, 1392 bp, synthetic construct
  source 1..1392: mol_type = other DNA
atgacccggctgaccgtgctggccctcctggctggactgctggcctcctcaagagcccaggtccagctggtgcaatccggcgccgaagtcaagaagccaggcgcaagcgtgaaagtgtcatgcaaagcctccggatacaccttcacctccacctggattcactgggtcagacaggcccccggtcaaggactggaatggatcgggctgatcgacccgtcggactcgtacaccaactacaatcagaagtttcgcggtcgggctactctcactgtggatacctcgacctccaccgcttacatggaactgtcatcgctgcggtccgaggataccgccgtgtactattgcgcgtcctccaagccgactggcggatggggacagggaactactgtgacggtgtcctccgcctcgaccaagggcccctccgtgtttccactggccccctcatccaagtctaccagcggaggaaccgcagccctaggctgtctcgtgaaggactacttccccgagccggtcactgtctcctggaactcgggagccctcactagcggtgtccacactttcccggcggtgttgcaaagctccgggctgtactccctgtcctcggtcgtcaccgtgccgtcaagctccctcgggacccagacatacatctgtaacgtcaaccataagccatccaacaccaaagtggacaagaaagtggagccgaaaagctgcgacaagactcacacttgccctccttgccctgcacccgagcttctcggaggtcccagcgtgttcctgttcccgccgaagcccaaggacactctgatgattagccgcactcctgaggtcacctgtgtcgtggtggacgtgtcccatgaggaccctgaagtcaagttcaattggtacgtggacggcgtggaggtgcacaacgccaagaccaagccgagggaggagcagtacaactcgacctatcgcgtggtgtccgtgctcaccgtgctgcatcaggattggctgaacgggaaggagtataagtgcaaagtgtccaacaaggctttgccggcccctatcgaaaagaccattagcaaggccaaggggcagccaagggagcctcaagtgtacaccctgccgccttcgagagatgaactgaccaagaaccaagtgtccctcacgtgcctcgtgaagggcttctacccctccgatatcgcggtggaatgggaatccaacggacagcccgaaaacaactacaagaccacccctccggtgcttgatagcgacggctcgttcttcctgtactcgaagctgacagtggacaagtcacggtggcagcagggcaacgtgttctcatgctccgtgatgcacgaagcgttgcacaatcactacacccagaagtcgcttagcctgagccctggatag

SEQ ID NO: 244 - DNA, 717 bp, synthetic construct; identical to SEQ ID NO: 242
  source 1..717: mol_type = other DNA

SEQ ID NO: 245 - DNA, 1392 bp, synthetic construct
  source 1..1392: mol_type = other DNA
atgacccggctgaccgtgctggccctcctggctggactgctggcctcctcaagagcccaggtccagctggtgcaatccggcgccgaagtcaagaagccaggcgcaagcgtgaaagtgtcatgcaaagcctccggatacaccttcacctccacctggattcactgggtcagacaggcccccggtcaaggactggaatggatcgggctgatcgacccgtcggactcgtacaccaactacaatcagaagtttcgcggtcgggctactctcactgtggatacctcgacctccaccgcttacatggaactgtcatcgctgcggtccgaggataccgccgtgtactattgcgcgtcctccaagccgactggcggatggggacagggaactactgtgacggtgtcctccgcctcgaccaagggcccctccgtgtttccactggccccctcatccaagtctaccagcggaggaaccgcagccctaggctgtctcgtgaaggactacttccccgagccggtcactgtctcctggaactcgggagccctcactagcggtgtccacactttcccggcggtgttgcaaagctccgggctgtactccctgtcctcggtcgtcaccgtgccgtcaagctccctcgggacccagacatacatctgtaacgtcaaccataagccatccaacaccaaagtggacaagaaagtggagccgaaaagctgcgacaagactcacacttgccctccttgccctgcacccgaggcagcaggaggtcccagcgtgttcctgttcccgccgaagcccaaggacactctgatgattagccgcactcctgaggtcacctgtgtcgtggtggacgtgtcccatgaggaccctgaagtcaagttcaattggtacgtggacggcgtggaggtgcacaacgccaagaccaagccgagggaggagcagtacaactcgacctatcgcgtggtgtccgtgctcaccgtgctgcatcaggattggctgaacgggaaggagtataagtgcaaagtgtccaacaaggctttgccggcccctatcgaaaagaccattagcaaggccaaggggcagccaagggagcctcaagtgtacaccctgccgccttcgagagatgaactgaccaagaaccaagtgtccctcacgtgcctcgtgaagggcttctacccctccgatatcgcggtggaatgggaatccaacggacagcccgaaaacaactacaagaccacccctccggtgcttgatagcgacggctcgttcttcctgtactcgaagctgacagtggacaagtcacggtggcagcagggcaacgtgttctcatgctccgtgatgcacgaagcgttgcacaatcactacacccagaagtcgcttagcctgagccctggatag

SEQ ID NO: 246 - protein, 451 aa, synthetic construct
QVQLQQPGAELVRPGTSVKLSCKASGYTFTSYWIHWVKQRPGQGLEWIGLIDPSDSYTNYNQKFRGKATLTVDTSSSTAYMQLSSLTSEDSAVYYCASSDPTGCWGQGTTLTVSSAKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 247 - protein, 445 aa, synthetic construct
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGGWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 219 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDIVMTQAAFSNPVTPGTPASISCRSSKSLLHSSGITYLSWYLQKPGQSPQLLIYQMSNLASGVPDRFSSSGSGTDFTLKISRVEAEDVGVYYCAQNLELPLTFGQGTKLEIK

SEQ ID NO: 249 - protein, 19 aa, unidentified
  source 1..19: note = Mouse/Rat P2 peptide
YSMKETTMKIIPFNRLSIG

SEQ ID NO: 250 - protein, 19 aa, Oryctolagus cuniculus
YSMKKTTMKIIPLNRLSIG

SEQ ID NO: 251 - protein, 29 aa, Homo sapiens
TTMKIIPFNRLTIGEGQQHHLGGAKQVRP

SEQ ID NO: 252 - protein, 29 aa, Rattus norvegicus
TTMKIIPFNRLSIGDGQQHHMGGSKQVSV

SEQ ID NO: 253 - protein, 29 aa, Oryctolagus cuniculus
TTMKIIPLNRLSIGEGQQFHVGGAKQVRP

SEQ ID NO: 254 - protein, 439 aa, synthetic construct
QVQLQQSGPELVRPGTSVRMSCKASGYTFTRYWMHWVKQRPGQGLEWIGMIDPSNSEIRLNQKFLDKATLNVDKSSNTAYMHLSSLTSEDSGVYYCARADWDRDSWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 255 - protein, 219 aa, synthetic construct
RADAAPTVSIFPPSSEQLTSGGASVVCFLNNFYPKDINVKWKIDGSERQDGVLNSWTDQDSKDSTYSMSSTLTLTKDEYERHNSYTCEATHKTSTSPIVKSFNRNECDIVMSQSPSSLAVSAGEKVTMTCKSSQSLLNSRTRKNYLAWYQEKPGQSPKLLIYWASTRESGVPDRFTGSGSGTDFTLTISSVQAEDLAVYYCKQSYTLRTFGGGTKLEIK

SEQ ID NO: 256 - protein, 116 aa, synthetic construct
QVQLQQSGPELVRPGTSVRMSCKASGYTFTRYWMHWVKQRPGQGLEWIGMIDPSNSEIRLNQKFLDKATLNVDKSSNTAYMHLSSLTSEDSGVYYCARADWDRDSWGQGTTLTVSS

SEQ ID NO: 257 - protein, 112 aa, synthetic construct
DIVMSQSPSSLAVSAGEKVTMTCKSSQSLLNSRTRKNYLAWYQEKPGQSPKLLIYWASTRESGVPDRFTGSGSGTDFTLTISSVQAEDLAVYYCKQSYTLRTFGGGTKLEIK

SEQ ID NO: 258 - protein, 10 aa, synthetic construct
GYTFTRYWMH

SEQ ID NO: 259 - protein, 17 aa, synthetic construct
MIDPSNSEIRLNQKFLD

SEQ ID NO: 260 - protein, 7 aa, synthetic construct
ADWDRDS

SEQ ID NO: 261 - skipped

SEQ ID NO: 262 - protein, 17 aa, synthetic construct
KSSQSLLNSRTRKNYLA

SEQ ID NO: 263 - protein, 7 aa, synthetic construct
WASTRES

SEQ ID NO: 264 - protein, 8 aa, synthetic construct
KQSYTLRT

SEQ ID NO: 265 - protein, 191 aa, synthetic construct
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 266 - protein, 431 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 267 - protein, 102 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTII

SEQ ID NO: 268 - protein, 214 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 230 aa, synthetic construct
EVQLVESGGGLVQPGGSLRLSCAASGYDFTHYMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 270 - protein, 219 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECDIVMTQAAFSNPITLGTSASMSCRSSKSLLHSSGITYLSWYLQKPGQSPQLLIYQMSNLASGVPDRFSSSGSGTDFTLRISRVEAEDVGVYYCAQNLELPLTFGAGTKLELK

SEQ ID NO: 271 - protein, 30 aa, synthetic construct
  VARIANT 6..30: note = GGGGS repeats may be deleted
GGGGSGGGGSGGGGSGGGGSGGGGSGGGGS

(19) United States
(12) Patent Application Publication
(10) Pub. No.: US 2026/0109756 A1
(43) Pub. Date: Apr. 23, 2026

(54) MULTI-SPECIFIC ANTIGEN BINDING PROTEINS WHICH BIND HUMAN FIBRIN OR FIBRINOGEN YC DOMAIN AND VASCULAR ENDOTHELIAL GROWTH FACTOR AND METHODS OF USE

(71) Applicant: Therini Bio, Inc., Sacramento, CA (US)

(72) Inventors: Kenneth Flanagan, Alameda, CA (US); Mathias Rickert, Hayward, CA (US); Michael Quigley, San Carlos, CA (US); Jeffrey Stavenhagen, San Mateo, CA (US)

(21) Appl. No.: 19/117,771

(22) PCT Filed: Oct. 3, 2023
(86) PCT No.: PCT/US2023/075837

§ 371 Date: Apr. 2, 2025

Related U.S. Application Data
(60) Provisional application No. 63/412,836, filed on Oct. 3, 2022.

(51) Int. Cl. C07K 16/22; A61K 39/00; A61P 27/02; C07K 16/18

(52) U.S. Cl. CPC C07K 16/22; A61P 27/02; C07K 16/18; A61K 2039/505; A61K 2039/54; C07K 2317/24; C07K 2317/31; C07K 2317/52; C07K 2317/565; C07K 2317/732; C07K 2317/734; C07K 2317/76; C07K 2317/92; C07K 2317/94

(57) ABSTRACT

Described herein are multi-specific antigen binding proteins that bind human fibrin or fibrinogen yC domain and vascular endothelial growth factor, and methods of use thereof. In certain aspects, described herein are pharmaceutical compositions comprising the multi-specific antigen binding proteins that bind fibrin or fibrinogen yC domain and vascular endothelial growth factor. In certain aspects, the antibodies and methods described herein are used for treatment of disorders or conditions of the eye.

### CROSS REFERENCE TO RELATED APPLICATIONS

This application claims the benefit of and priority to U.S. Provisional Application No. 63/412,836, filed Oct. 3, 2022, the entire disclosure of which is hereby incorporated by reference in its entirety for all purposes.

### BACKGROUND

Microglia are resident immune cells of the central nervous system (CNS), including the retina. Microglia have been implicated in many degenerative eye disorders, including retinitis pigmentosa, age-related macular degeneration (AMD), glaucoma, diabetic retinopathy, uveitis, and retinal detachment. While these diseases differ in their underlying etiologies, they are all characterized by the loss of photoreceptors or retinal ganglion cells (RGCs), resulting in deterioration of vision and, in some cases, blindness. Activation of microglia contributes to both neuronal and oligodendrocyte death via release of cytokines and nitric oxide. It is possible that interventions targeting activated microglia could alleviate photoreceptor and RGC death, thereby helping patients preserve their sight.

The γ377-395 epitope of the fibrin or fibrinogen γC domain is the binding epitope of fibrin to CDIIb. The fibrin γ³⁷⁷⁻³⁹⁵ peptide functions as an inhibitor of microglia activation by blocking fibrin binding to Mac-1. Because fibrin mediates blood coagulation by binding via a distinct epitope to the platelet integrin α_{IIb}β₃ receptor, therapeutic agents (including antibodies), that block the CD11b binding epitope to fibrin can reduce the damaging effects of fibrin in the nervous system without affecting its beneficial effects in blood coagulation.

Angiogenic ocular conditions such as retinopathy of prematurity, diabetic retinopathy, and age-related macular degeneration represent the leading cause of irreversible vision loss in developed countries. Evidence suggests that vascular endothelial growth factor (VEGF) promotes angiogenesis in each of these conditions.

Therefore, safe, effective therapeutics that inhibit fibrin-induced microglial activation without affecting its beneficial effects in blood coagulation, and that also block VEGF, are needed for treatment of disorders and conditions of the eye.

### SUMMARY

In certain aspects, described herein are multi-specific antigen binding proteins comprising at least two distinct antigen binding regions, wherein a first antigen binding region specifically binds the human fibrin or fibrinogen γC domain, and a second antigen binding region specifically binds vascular endothelial growth factor (VEGF).

In certain embodiments, the first antigen binding region comprises a heavy chain comprising a variable heavy (VH) chain sequence comprising three heavy chain CDR sequences, CDR-H1, CDR-H2, and CDR-H3, and a light chain comprising a variable light (VL) chain sequence comprising three light chain CDR sequences, CDR-L1, CDR-L2, and CDR-L3, wherein: CDR-H1 comprises the sequence set forth in SEQ ID NOs: 1, 13, 25, 37, 49, 61, 73, 85, 97, 109, 121, 133, 145, 157, 169, 181, 193, 205, 217, 229, or 258; CDR-H2 comprises the sequence set forth in SEQ ID NOs: 2, 14, 26, 38, 50, 62, 74, 86, 98, 110, 122, 134, 146, 158, 170, 182, 194, 206, 218, 230, or 259; CDR-H3 comprises the sequence set forth in SEQ ID NOs: 3, 15, 27, 39, 51, 63, 75, 87, 99, 111, 123, 135, 147, 159, 171, 183, 195, 207, 219, 231, or 260; CDR-L1 comprises the sequence set forth in SEQ ID NOs: 4, 16, 28, 40, 52, 64, 76, 88, 100, 112, 124, 136, 148, 160, 172, 184, 196, 208, 220, 232, or 262; CDR-L2 comprises the sequence set forth in SEQ ID NOs: 5, 17, 29, 41, 53, 65, 77, 89, 101, 113, 125, 137, 149, 161, 173, 185, 197, 209, 221, 233, or 263; and CDR-L3 comprises the sequence set forth in SEQ ID NOs: 6, 18, 30, 42, 54, 66, 78, 90, 102, 114, 126, 138, 150, 162, 174, 186, 198, 210, 222, 234 or 264.

In certain embodiments, the multi-specific antigen binding protein comprises a VH sequence selected from a sequence set forth in one of SEQ ID NOs: 7, 19, 31, 43, 55, 67, 79, 91, 103, 115, 127, 139, 151, 163, 175, 187, 199, 211, 223, or 235.

In certain embodiments, the multi-specific antigen binding protein comprises a VL sequence selected from a sequence set forth in SEQ ID NOs: 10, 22, 34, 46, 58, 70, 82, 94, 106, 118, 130, 142, 154, 166, 178, 190, 202, 214, 226, or 238.

In certain embodiments, the first antigen binding region comprises a VH sequence set forth in one of SEQ ID NO: 7 and a VL sequence set for in SEQ ID NO: 10. In certain embodiments, the first antigen binding region comprises a VH sequence set forth in SEQ ID NO: 19 and a VL sequence set forth in SEQ ID NO: 22. In certain embodiments, the first antigen binding region comprises a VH sequence set forth in SEQ ID NO: 31 and a VL sequence set forth in SEQ ID NO: 34. In certain embodiments, the first antigen binding region comprises a VH sequence set forth in SEQ ID NO: 43 and a VL sequence set forth in SEQ ID NO: 46. In certain embodiments, the first antigen binding region comprises a VH sequence set forth in SEQ ID NO: 55 and a VL sequence set forth in SEQ ID NO: 58. In certain embodiments, the first antigen binding region comprises a VH sequence set forth in SEQ ID NO: 67 and a VL sequence set forth in SEQ ID NO: 70. In certain embodiments, the first antigen binding region comprises a VH sequence set forth in SEQ ID NO: 79 and a VL sequence set forth in SEQ ID NO: 82. In certain embodiments, the first antigen binding region comprises a VH sequence set forth in SEQ ID NO: 91 and a VL sequence set forth in SEQ ID NO: 94. In certain embodiments, the first antigen binding region comprises a VH sequence set forth in SEQ ID NO: 103 and a VL sequence set forth in SEQ ID NO: 106. In certain embodiments, the first antigen binding region comprises a VH sequence set forth in SEQ ID NO: 115 and a VL sequence set forth in SEQ ID NO: 118. In certain embodiments, the first antigen binding region comprises a VH sequence set forth in SEQ ID NO: 127 and a VL sequence set forth in SEQ ID NO: 130. In certain embodiments, the first antigen binding region comprises a VH sequence set forth in SEQ ID NO: 139 and a VL sequence set forth in SEQ ID NO: 142. In certain embodiments, the first antigen binding region comprises a VH sequence set forth in SEQ ID NO: 151 and a VL sequence set forth in SEQ ID NO: 154. In certain embodiments, the first antigen binding region comprises a VH sequence set forth in SEQ ID NO: 163 and a VL sequence set forth in SEQ ID NO: 166. In certain embodiments, the first antigen binding region comprises a VH sequence set forth in SEQ ID NO: 175 and a VL sequence set forth in SEQ ID NO: 178. In certain embodiments, the first antigen binding region comprises a VH sequence set forth in SEQ ID NO: 187 and a VL sequence set forth in SEQ ID NO: 190. In certain embodiments, the first antigen binding region comprises a VH sequence set forth in SEQ ID NO: 199 and a VL sequence set forth in SEQ ID NO: 202. In certain embodiments, the first antigen binding region comprises a VH sequence set forth in SEQ ID NO: 211 and a VL sequence set forth in SEQ ID NO: 214. In certain embodiments, the first antigen binding region comprises a VH sequence set forth in SEQ ID NO: 223 and a VL sequence set forth in SEQ ID NO: 226. In certain embodiments, the first antigen binding region comprises a VH sequence set forth in SEQ ID NO: 235 and a VL sequence set forth in SEQ ID NO: 238. In certain embodiments, the first antigen binding region comprises the VH sequence set forth in SEQ ID NO: 256 and the VL sequence set forth in SEQ ID NO: 257.

In certain embodiments, the first antigen binding region comprises a humanized, human, or chimeric antibody. In certain embodiments, the first antigen binding region comprises a humanized antibody. In certain embodiments, the first antigen binding region comprises a heavy chain human constant region of a class selected from IgG, IgA, IgD, IgE, and IgM. In certain embodiments, the human Fc region comprises a human heavy chain constant region of the class IgG and a subclass selected from IgG1, IgG2, IgG3, and IgG4. In certain embodiments, the human Fc region comprises wild-type, human IgG1 Fc. In certain embodiments, the human Fc domain comprises a sequence set forth in SEQ ID NOs: 8, 20, 32, 44, 56, 68, 80, 92, 104, 116, 128, 140, 152, 164, 176, 188, 200, 212, 224, or 236.

In certain embodiments, the heavy chain comprises a constant heavy chain sequence set forth by SEQ ID NOs: 8, 20, 32, 44, 56, 68, 80, 92, 104, 116, 128, 140, 152, 164, 176, 188, 200, 212, 224, or 236. In certain embodiments, the light chain comprises a constant light chain sequence set forth by SEQ ID NOs: 9, 21, 33, 45, 57, 69, 81, 93, 105, 117, 129, 141, 153, 165, 177, 189, 201, 213, 225, or 237.

In certain embodiments, the first antigen binding region comprises the VH sequence set forth in SEQ ID NO: 7, and the VL sequence set forth in SEQ ID NO: 10; and the human Fc region comprises wild-type, human IgG1 Fc. In certain embodiments, the first antigen binding region comprises the VH sequence set forth in SEQ ID NO: 19, and the VL sequence set forth in SEQ ID NO: 22; and the human Fc region comprises wild-type, human IgG1 Fe. In certain embodiments, the first antigen binding region comprises the VH sequence set forth in SEQ ID NO: 31, and the VL sequence set forth in SEQ ID NO: 34; and the human Fc region comprises wild-type, human IgG1 Fe. In certain embodiments, the first antigen binding region comprises the VH sequence set forth in SEQ ID NO: 43, and the VL sequence set forth in SEQ ID NO: 46; and the human Fc region comprises wild-type, human IgG1 Fe. In certain embodiments, the first antigen binding region comprises the VH sequence set forth in SEQ ID NO: 55, and the VL sequence set forth in SEQ ID NO: 58; and the human Fe region comprises wild-type, human IgG1 Fc. In certain embodiments, the first antigen binding region comprises the VH sequence set forth in SEQ ID NO: 67, and the VL sequence set forth in SEQ ID NO: 70; and the human Fc region comprises wild-type, human IgG1 Fc. In certain embodiments, the first antigen binding region comprises the VH sequence set forth in SEQ ID NO: 79, and the VL sequence set forth in SEQ ID NO: 82; and the human Fe region comprises wild-type, human IgG1 Fc. In certain embodiments, the first antigen binding region comprises the VH sequence set forth in SEQ ID NO: 91, and the VL sequence set forth in SEQ ID NO: 94; and the human Fc region comprises wild-type, human IgG1 Fc. In certain embodiments, the first antigen binding region comprises the VH sequence set forth in SEQ ID NO: 103, and the VL sequence set forth in SEQ ID NO: 106; and the human Fe region comprises wild-type, human IgG1 Fc. In certain embodiments, the first antigen binding region comprises the VH sequence set forth in SEQ ID NO: 115, and the VL sequence set forth in SEQ ID NO: 118; and the human Fc region comprises wild-type, human IgG1 Fc. In certain embodiments, the first antigen binding region comprises the VH sequence set forth in SEQ ID NO: 127, and the VL sequence set forth in SEQ ID NO: 130; and the human Fc region comprises wild-type, human IgG1 Fe. In certain embodiments, the first antigen binding region comprises the VH sequence set forth in SEQ ID NO: 139, and the VL sequence set forth in SEQ ID NO: 142; and the human Fc region comprises wild-type, human IgG1 Fe. In certain embodiments, the first antigen binding region comprises the VH sequence set forth in SEQ ID NO: 151, and the VL sequence set forth in SEQ ID NO: 154; and the human Fc region comprises wild-type, human IgG1 Fe. In certain embodiments, the first antigen binding region comprises the VH sequence set forth in SEQ ID NO: 163, and the VL sequence set forth in SEQ ID NO: 166; and the human Fc region comprises wild-type, human IgG1 Fe. In certain embodiments, the first antigen binding region comprises the VH sequence set forth in SEQ ID NO: 175, and the VL sequence set forth in SEQ ID NO: 178; and the human Fc region comprises wild-type, human IgG1 Fe. In certain embodiments, the first antigen binding region comprises the VH sequence set forth in SEQ ID NO: 187, and the VL sequence set forth in SEQ ID NO: 190; and the human Fc region comprises wild-type, human IgG1 Fe. In certain embodiments, the first antigen binding region comprises the VH sequence set forth in SEQ ID NO: 199, and the VL sequence set forth in SEQ ID NO: 202; and the human Fc region comprises wild-type, human IgG1 Fe. In certain embodiments, the first antigen binding region comprises the VH sequence set forth in SEQ ID NO: 211, and the VL sequence set forth in SEQ ID NO: 214; and the human Fc region comprises wild-type, human IgG1 Fe. In certain embodiments, the first antigen binding region comprises the VH sequence set forth in SEQ ID NO: 223, and the VL sequence set forth in SEQ ID NO: 226; and the human Fc region comprises wild-type, human IgG1 Fe. In certain embodiments, the first antigen binding region comprises the VH sequence set forth in SEQ ID NO: 235, and the VL sequence set forth in SEQ ID NO: 238; and the human Fc region comprises wild-type, human IgG1 Fe. In certain embodiments, the first antigen binding region comprises the VH sequence set forth in SEQ ID NO: 256, and the VL sequence set forth in SEQ ID NO: 257; and the human Fc region comprises wild-type, human IgG1 Fc.

In certain embodiments, the Fc region comprises one or more amino acid substitutions, wherein the one or more substitutions result in increased half-life, increased ADCC activity, increased ADCP activity, or increased CDC activity compared with the Fc without the one or more substitutions. In certain embodiments, the Fc region binds an Fcγ receptor selected from the group consisting of: FcγRI, FcγRIIa, FcγRIIb, FcγRIIc, FcγRIIIa, and FcγRIIIb.

In certain embodiments, the first antigen binding region comprises a monoclonal antibody. In certain embodiments, the first antigen binding region consists of a monoclonal antibody. In certain embodiments, the first antigen binding region binds an γ377-395 epitope of the fibrin or fibrinogen γC domain. In certain embodiments, the first antigen binding region binds to a peptide comprising an amino acid sequence set forth in at least one of SEQ ID NOs: 241 and 249-253 with a K_{D} of less than or equal to about 1, 2, 3, 4, 5, 6, 7, or 8×10⁻⁵ M, as measured by a surface plasmon resonance (SPR) single cycle kinetics (SCK) assay. In certain embodiments, the first antigen binding region binds to a peptide comprising the sequence of the γ377-395 epitope of the human fibrin or fibrinogen γC domain with a K_{D} of less than or equal to about 8×10⁻⁵ M, as measured by a surface plasmon resonance (SPR) single cycle kinetics (SCK) assay. In certain embodiments, the first antigen binding region inhibits Mac-1 binding to fibrin or fibrinogen γC domain. In certain embodiments, the first antigen binding region exhibits inhibition of microglial adhesion to the fibrin or fibrinogen γC domain.

In certain embodiments, the first antigen binding region binds human fibrin at any one of amino acid residues Lys 411, Ile 412, Ile 413, Phe 415, Asn 416, Arg 417, Leu 418, Thr 419, Ile 420, and Gly 421. In certain embodiments, the first antigen binding region binds human fibrin at least two, three, four, five, six, seven, eight, nine, or all ten of amino acid residues Lys 411, Ile 412, Ile 413, Phe 415, Asn 416, Arg 417, Leu 418, Thr 419, Ile 420, and Gly 421. In certain embodiments, the first antigen binding region comprises a VH region comprising a paratope that comprises any one of amino acid residues Ser 31, Tyr 32, Trp 33, His 35, Trp 47, Leu 50, Asp 52, Asp 54, Tyr 56, Ala 93, Ser 94, Ser 95, Lys 96 or Asp 96, Pro 97 or Ala 97, Gly 101, Gly 102, and Trp 103. In certain embodiments, the first antigen binding region comprises a VH region comprising a paratope that comprises at least two, three, four, five, six, seven, eight, nine, ten, eleven, twelve, thirteen, fourteen, fifteen, sixteen, or all seventeen of amino acid residues Ser 31, Tyr 32, Trp 33, His 35, Trp 47, Leu 50, Asp 52, Asp 54, Tyr 56, Ala 93, Ser 94, Ser 95, Lys 96 or Asp 96, Pro 97 or Ala 97, Gly 101, Gly 102, and Trp 103. In certain embodiments, the first antigen binding region comprises a VH region comprising a paratope that comprises amino acid residues Ser 31, Trp 33, His 35, Asp 52, Asp 54, Tyr 56, Ser 94, Gly 101, Gly 102, and Trp 103. In certain embodiments, the first antigen binding region comprises a VH region comprising a paratope that comprises amino acid residues Ser 31, Trp 33, His 35, Asp 52, Asp 54, Tyr 56, Ala 93, Ser 94, Lys 96, Pro 97, Gly 101, Gly 102, and Trp 103. In certain embodiments, the first antigen binding region comprises a VH region comprising a paratope that comprises amino acid residues Ser 31, Tyr 32, Trp 33, His 35, Trp 47, Asp 52, Asp 54, Tyr 56, Ser 94, Ser 95, Asp 96, Ala 97, Gly 101, Gly 102, and Trp 103. In certain embodiments, the first antigen binding region comprises a VL region comprising a paratope that comprises any one of amino acid residues His 27, Tyr 32, Tyr 36, Leu 46, Tyr 49, Gln 50, Ala 91 or Asn 91, Leu 92, Leu 94, and Leu 96. In certain embodiments, the first antigen binding region comprises a VL region comprising a paratope that comprises at least two, three, four, five, six, seven, eight, nine, or all ten amino acid residues His 27, Tyr 32, Tyr 36, Leu 46, Tyr 49, Gln 50, Ala 91 or Asn 91, Leu 92, Leu 94, and Leu 96. In certain embodiments, the first antigen binding region comprises a VL region comprising a paratope that comprises the amino acid residues His 27, Tyr 32, Tyr 36, Leu 46, Gln 50, Leu 92, Leu 94, and Leu 96. In certain embodiments, the first antigen binding region comprises a VL region comprising a paratope that comprises the amino acid residues His 27, Tyr 32, Tyr 36, Leu 46, Gln 50, Asn 91, Leu 92, Leu 94, and Leu 96. In certain embodiments, the first antigen binding region comprises a VL region comprising a paratope that comprises the amino acid residues His 27, Tyr 32, Tyr 36, Leu 46, Tyr 49, Gln 50, Ala 91, Leu 92, Leu 94, and Leu 96. In certain embodiments, the first antigen binding region comprises a humanized, human, or chimeric antibody.

In certain embodiments, the first antigen binding region comprises a humanized antibody. In certain embodiments, the first antigen binding region comprises a heavy chain human constant region of a class selected from IgG, IgA, IgD, IgE, and IgM. In certain embodiments, the human Fc region comprises a human heavy chain constant region of the class IgG and a subclass selected from IgG1, IgG2, IgG3, and IgG4. In certain embodiments, the human Fc region comprises wild-type, human IgG1 Fc. In certain embodiments, the human Fc domain comprises a sequence set forth in SEQ ID NOs: 8, 20, 32, 44, 56, 68, 80, 92, 104, 116, 128, 140, 152, 164, 176, 188, 200, 212, 224, or 236. In certain embodiments, the Fc region comprises one or more amino acid substitutions, wherein the one or more substitutions results in increased half-life, increased ADCC activity, increased ADCP activity, or increased CDC activity compared with the Fc without the one or more substitutions. In certain embodiments, the Fc region binds an Fcγ receptor selected from the group consisting of: FcγRI, FcγRIIa, FcγRIlb, FcγRIIc, FcγRIIIa, and FcγRIIIb.

In certain embodiments of the multi-specific antigen binding proteins described herein, the second antigen binding region comprises a heavy chain comprising a second VH chain sequence comprising three heavy chain CDR sequences, CDR-H1, CDR-H2, and CDR-H3, and a light chain comprising a second VL chain sequence comprising three light chain CDR sequences, CDR-L1, CDR-L2, and CDR-L3.

In certain embodiments, the second antigen binding region binds one or more of VEGF-A, VEGF-B, VEGF-C, and VEGF-D. In certain embodiments, the second antigen binding region comprises a VH amino acid sequence that is 90% or more, 95% or more, or 99% or more identical to the VH amino acid sequence set forth in SEQ ID NO: 269. In certain embodiments, the second antigen binding region comprises a VL amino acid sequence that is 90% or more, 95% or more, or 99% or more identical to the VL amino acid sequence set forth in SEQ ID NO: 268. In certain embodiments, the second antigen binding region binds an epitope of VEGF-A comprising at least one of amino acids 82-91 set forth in SEQ ID NO. 265. In certain embodiments, the second antigen binding region comprises a sequence that is 90% or more, 95% or more, or 99% or more identical to the amino acid sequence set forth in SEQ ID NOs: 266 or 267. In certain embodiments, the second antigen binding region does not comprise an Fc domain.

In certain embodiments, the multi-specific antigen binding protein comprises a heterodimeric Fc domain. In certain embodiments, the multi-specific antigen binding protein comprises a bi-specific antibody. In certain embodiments, the multi-specific antigen binding protein consists of a bi-specific antibody. In certain embodiments, the multi-specific antigen binding protein comprises one or more additional antigen binding domains that specifically bind to human fibrin or fibrinogen γC domain. In certain embodiments, the multi-specific antigen binding protein comprises one or more additional antigen binding domains that specifically bind to VEGF.

In certain embodiments, the multi-specific antigen binding protein is used in the treatment of a disorder or condition of eye. In certain embodiments, the multi-specific antigen binding protein is formulated for administration to a subject by intravitreal injection.

In certain aspects, described herein are isolated polynucleotides or sets of polynucleotides encoding a first antigen binding region, a VH thereof, a VL thereof, a light chain thereof, a heavy chain thereof, or an antigen-binding portion thereof; optionally wherein the polynucleotide or set of polynucleotides comprises cDNA. In certain aspects, described herein are isolated polynucleotides or sets of polynucleotides encoding a second antigen binding region, a VH thereof, a VL thereof, a light chain thereof, a heavy chain thereof, or an antigen-binding portion thereof; optionally wherein the polynucleotide or set of polynucleotides comprises cDNA.

In certain aspects, described herein are vectors or sets of vectors comprising the polynucleotide or set of polynucleotides described herein.

In certain aspects, described herein is a host cell comprising the polynucleotide or set of polynucleotides or the vector or set of vectors described herein.

In certain aspects, described herein are methods of producing a multi-specific antigen binding protein described herein, the method comprising expressing the multi-specific antigen binding protein or antigen binding region thereof in the host cell described herein and isolating the expressed multi-specific antigen binding protein or antigen binding region thereof.

In certain aspects, described herein are pharmaceutical compositions comprising an multi-specific antigen binding protein described herein and a pharmaceutically acceptable excipient.

In certain aspects, described herein are kits comprising the multi-specific antigen binding protein described herein or a pharmaceutical composition described herein and instructions for use.

In certain aspects, described herein are methods for treating a disorder or condition of the eye, the method comprising administering to a mammalian subject a therapeutically effective amount the multi-specific antigen binding protein described herein or a pharmaceutical composition described herein. In certain embodiments, the disorder or condition of the eye is selected from the group consisting of: retinitis pigmentosa, age-related macular degeneration, glaucoma, diabetic retinopathy, uveitis, and retinal detachment.

In certain aspects, described herein are methods for treating a pathology associated with Mac-1 binding to fibrin or Mac-1 binding with fibrinogen, the method comprising administering to a mammalian subject a therapeutically effective amount the multi-specific antigen binding protein described herein or a pharmaceutical composition thereof described herein.

In certain aspects, described herein are methods of inhibiting microglia activation, the method comprising administering to a mammalian subject a therapeutically effective amount of multi-specific antigen binding protein described herein or a pharmaceutical composition thereof described herein.

In certain embodiments, described herein are methods of producing a multi-specific antigen binding protein, the method comprising expressing a multi-specific antigen binding protein or antigen binding region thereof in the host cell described herein and isolating the expressed multi-specific antigen binding protein or antigen binding region thereof.

In certain aspects, described herein are methods of preventing a disorder or condition of the eye, the method comprising administering to a mammalian subject a therapeutically effective amount the multi-specific antigen binding protein described herein or the pharmaceutical composition thereof described herein.

In certain aspects, described herein are methods of treating a disorder or condition of the eye associated with increased vascularization in a subject in need thereof, comprising administering to the subject the multi-specific antigen binding protein described herein or a pharmaceutical composition thereof described herein.

A method of preventing a disorder or condition of the eye associated with increased vascularization in a subject in need thereof, comprising administering to the subject the multi-specific antigen binding protein described herein or a pharmaceutical composition thereof described herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a graph depicting the uveitis clinical score of rats administered intravitreally isotype control, murinized antibody clone 60143—with Fc stabilization LALA mutations (low dose=10 μg/eye; high dose=50 μg/eye), positive control FTY-720 (administered by oral gavage at a dose of 0.3 mg/kg) and naïve mice with no experimental autoimmune encephalomyelitis (EAE) induction.

FIG. 2 is a graph showing vascular leakage via quantitative fluorescein angiography (qFA) in rats treated with the anti-VEGF antigen binding construct Eylea (aflibercept) and murinized anti-fibrin antibody clone 60143—with Fc stabilization LALA mutations (“m60143-LALA”) at the indicated concentrations.

FIG. 3 is a graph showing choroidal neovascularization (CNV) lesion area in rats treated with the anti-VEGF antigen binding construct Eylea (aflibercept) and murinized anti-fibrin antibody clone 60143—with Fc stabilization LALA mutations (“m60143-LALA”) at the indicated concentrations.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a,” “an,” and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-FIBRIN antibody, administered to an individual, either as a single dose or as part of a series of doses, which is effective to produce or contribute to a desired therapeutic effect, either alone or in combination with another therapeutic modality. Examples of a desired therapeutic effect is enhancing an immune response, slowing or delaying tumor development; stabilization of disease; amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the term “subject” or “individual” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody provided herein. In some aspects, the disease or condition is a cancer. In some aspects, the disease or condition is a viral infection.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “co-administration,” “co-administer,” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, therapeutic agents are in the same composition or unit dosage form. In other embodiments, therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ. In some embodiments, the affinity of an antibody is described in terms of the K_{D} for an interaction between such antibody and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular antibody-antigen interaction. K_{A}=kₐ/k_{d}.

The term “multi-specific antigen binding protein” is used herein in its broadest sense to include molecules comprising polypeptides that have the capability of binding to two or more distinct antigens. Multi-specific antigen binding proteins include multi-specific antibodies and antibodies comprising binding agents that have affinity to one or more antigens.

The term “antigen binding region” or “antigen-binding region” is used herein in its broadest sense to include a region of a multi-specific antigen binding protein comprising a polypeptide comprising affinity to an antigen or epitope.

The term “fibrin antigen binding region” refers to an antigen binding region with affinity to fibrin and includes fibrin antibodies.

The term “VEGF antigen binding region” refers to an antigen binding region with affinity to one or more of VEGF-A, VEGF-B, VEGF-C, and VEGF-D.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. Antibodies include any alternative antibody format known in the art including, but not limited to: single domain antibodies, diabodies, knobs-into-hole antibodies, scFv, scFv dimers, BsFv, dsFv, a(dsFv)2, dsFv-dsFv′, Fv fragments, Fab, Fab′, F(ab′)2, ds diabodies, minibodies, nanobodies, domain antibodies, or a bivalent domain antibody.

A “fibrin antibody,” “anti-fibrin antibody,” or “fibrin-specific antibody” is an antibody, as provided herein, which specifically binds to the antigen fibrin. In some embodiments, the antibody binds the extracellular domain of fibrin. In certain embodiments, a fibrin antibody provided herein binds to an epitope of fibrin that is conserved between or among fibrin proteins from different species.

The term “epitope” means a portion of an antigen that specifically binds to an antibody.

The term “hypervariable region” or “HVR,” as used herein, refers to each of the regions of an antibody variable domain which are hypervariable in sequence and/or form structurally defined loops (“hypervariable loops”).

The term “chimeric antibody” refers to an antibody in which a portion of the heavy and/or light chain is derived from a particular source or species, while the remainder of the heavy and/or light chain is derived from a different source or species.

The term “human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” or “multi-specific antibody” refers to an antibody that comprises two or more different antigen-binding regions that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e., a native cell, or host cell in the case of recombinantly produced heteromultimer that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

### Multi-Specific Antigen Binding Proteins

The present application provides multi-specific antigen binding proteins which comprises at least two distinct antigen binding domains; wherein at least one antigen binding domain binds human fibrin or fibrinogen γC domain, and at least one antigen binding domain binds at least one isoform of VEGF.

Multi-specific antigen binding proteins of the present disclosure can comprise any antigen binding domain that binds to the antigen including, without limitation, a monoclonal antibody, a polyclonal antibody, a recombinant antibody, a bispecific antibody, a conjugated antibody, a human antibody, a humanized antibody, and a functional fragment thereof, including but not limited to a single-domain antibody (sdAb) such as a heavy chain variable domain (VH), a light chain variable domain (VL) and a variable domain (VHH) of camelid derived nanobody, and to an alternative scaffold known in the art to function as antigen-binding domain, such as a recombinant fibronectin domain, a T cell receptor (TCR), a recombinant TCR with enhanced affinity, or a fragment thereof, e.g., single chain TCR, and the like. In some instances, it is beneficial for the antigen-binding domain to be derived from the same species in which the multi-specific antigen binding protein will ultimately be used in. For example, for use in humans, it may be beneficial for the antigen-binding domain of the multi-specific antigen binding protein to comprise human or humanized residues for the antigen-binding domain of an antibody or antibody fragment.

In some embodiments, the antigen-binding domain of the multi-specific antigen binding protein comprises an antibody. In certain embodiments, the antibody is a human antibody. In certain embodiments, the antibody is a humanized antibody. In certain embodiments, the antibody is a chimeric antibody. In some embodiments, the antigen-binding domain comprises an antigen-binding fragment of an antibody. In some embodiments, the antigen-binding domain comprises a F(ab) fragment. In certain embodiments, the antigen-binding domain comprises a F(ab′) fragment. In some embodiments, the antigen-binding domain comprises an scFv. In some embodiments, the antigen-binding domain comprises two single chain variable fragments (scFvs). In some embodiments, each of the two scFvs binds to a distinct epitope on the same antigen. In some embodiments, the antigen-binding domain comprises a first scFv and a second scFv. In some embodiments, the first scFv and the second scFv bind distinct epitopes on the same antigen. In certain embodiments, the scFv is a human scFv. In certain embodiments, the scFv is a humanized scFv. In certain embodiments, the scFv is a chimeric scFv. In certain embodiments, the scFv comprises a heavy chain variable domain (VH) and a light chain variable domain (VL). In certain embodiments, the VH and VL are separated by a peptide linker. In certain embodiments, the scFv comprises the structure VH-L-VL or VL-L-VH, wherein VH is the heavy chain variable domain, L is the peptide linker, and VL is the light chain variable domain.

In some embodiments, each of the one or more scFvs comprises the structure VH-L-VL or VL-L-VH, wherein VH is the heavy chain variable domain, L is the peptide linker, and VL is the light chain variable domain. When there are two or more scFv linked together, each scFv can be linked to the next scFv with a peptide linked. In some embodiments, each of the one or more scFvs is separated by a peptide linker. In some embodiments, each of the two scFvs binds to a distinct epitope on the same antigen.

In some embodiments, the antigen binding domain comprises a single-domain antibody (sdAb). In certain embodiments, the sdAb is a humanized sdAb. In certain embodiments, the sdAb is a chimeric sdAb.

In some embodiments, a multi-specific antigen binding protein of the present disclosure may comprise two or more antigen-binding domains, three or more antigen-binding domains, four or more antigen-binding domains, five or more antigen-binding domains, six or more antigen-binding domains, seven or more antigen-binding domains, eight or more antigen-binding domains, nine or more antigen-binding domains, or ten or more antigen-binding domains. In some embodiments, each of the two or more antigen-binding domains binds the same antigen. In some embodiments, each of the two or more antigen-binding domains binds a different epitope of the same antigen. In some embodiments, each of the two or more antigen-binding domains binds a different antigen. In some embodiments, the two or more antigen-binding domains provide the multi-specific antigen binding protein with logic gating, such as ‘or’ logic gating.

In some embodiments, the multi-specific antigen binding protein comprises two antigen-binding domains. In some embodiments, the two antigen-binding domains are attached to one another via a flexible linker. In some embodiments, each of the two-antigen-binding domains may be independently selected from an antibody, an antigen-binding fragment of an antibody, an scFv, a sdAb, or a recombinant fibronectin domain.

In certain embodiments, the multi-specific antigen binding protein comprises an antigen-binding domain comprising a bispecific antibody or antibody fragment (e.g., scFv). In some embodiments, within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁ VH₁-VH₂-VL₂. In some embodiments, a linker is disposed between the two antibodies or antibody fragments (e.g., scFvs), for example, between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker, wherein n is 1, 2, 3, 4, 5, or 6. In general, the linker between the two scFvs should be long enough to avoid mispairing between the domains of the two scFvs. In some embodiments, a linker is disposed between the VL and VH of the first scFv. In some embodiments, a linker is disposed between the VL and VH of the second scFv. In constructs that have multiple linkers, any two or more of the linkers may be the same or different. Accordingly, in some embodiments, a multi-specific antigen binding protein comprises VLs, VHs, and may further comprise one or more linkers in an arrangement as described herein.

The recognized immunoglobulin genes include the kappa, lambda, alpha, gamma, delta, epsilon, and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and TgM, and several of these may be further divided into subclasses (isotypes), e.g., IgG1, IgG2, IgG3, IgG4, IgA₁, and IgA₂. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2 and CH3 domains respectively from the N to C-terminus. The light chain comprises of the VL and CL domains from N to C terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or derived from an immunoglobulin based construct such as a diabody, or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

In some embodiments, the multi-specific antigen binding proteins provided herein comprise one or more heavy chains. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, the multi-specific antigen binding protein comprises an IgG1 antibody. In some embodiments, the multi-specific antigen binding protein comprises an IgG3 antibody. In some embodiments, the multi-specific antigen binding protein comprises an IgG2 antibody. In some embodiments, the multi-specific antigen binding protein comprises an IgG4 antibody.

Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

Table A provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE A
Table A. Residues in CDRs according to
Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat | H31-H35B | H26-H32 |
| Numbering) | | or H34* |
| H1 (Chothia | H31-H35 | H26-H32 |
| Numbering) | | |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to Fibrin variants with different point-mutations, or to chimeric Fibrin variants.

To screen for antibodies which bind to an epitope on a target antigen bound by an antibody of interest (e.g., Fibrin), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human antibodies are antibodies which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

The two or more different epitopes may be epitopes on the same antigen (e.g., a single Fibrin molecule expressed by a cell) or on different antigens (e.g., different Fibrin molecules expressed by the same cell, or a Fibrin molecule and a non-Fibrin molecule). In some aspects, a multi-specific antibody binds two different epitopes (i.e., a “bispecific antibody” or “bi-specific antibody”). In some aspects, a multi-specific antibody binds three different epitopes (i.e., a “trispecific antibody”).

The antigen binding domains of the multi-specific antigen binding proteins can include an antibody or variable domain described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments the multi-specific antigen binding protein comprises a monoclonal antibody.

In some embodiments the multi-specific antigen binding protein or antigen binding region thereof is produced by hybridomas. In other embodiments, the antibodies are produced by recombinant cells engineered to express the desired variable and constant domains.

In some embodiments the multi-specific antigen binding protein comprise one or more single chain antibodies or other antibody derivatives retaining the antigen specificity and the lower hinge region or a variant thereof.

In some embodiments the multi-specific antigen binding protein may be polyfunctional antibodies, recombinant antibodies, human antibodies, humanized antibodies, fragments or variants thereof. In particular embodiments, the antibody fragment or a derivative thereof is selected from a Fab fragment, a Fab′2 fragment, a CDR and scFv.

In some embodiments, the antibodies are capable of forming an immune complex. For example, an immune complex can be a tumor cell covered by antibodies.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### Fibrin Antigen Binding Regions

### V_{H} Domains

In some embodiments, a fibrin antigen binding region provided herein comprises a V_{H} sequence selected from SEQ ID NOs: 7, 8, 9, 10, 11, 12, 13, 14, 15, 16, 17, 18, 19, and 20. In some embodiments, an antibody provided herein comprises a V_{H} sequence of SEQ ID NO: 7. In some embodiments, an antibody provided herein comprises a V_{H} sequence of SEQ ID NO: 8. In some embodiments, an antibody provided herein comprises a V_{H} sequence of SEQ ID NO: 9. In some embodiments, an antibody provided herein comprises a V_{H} sequence of SEQ ID NO: 10. In some embodiments, an antibody provided herein comprises a V_{H} sequence of SEQ ID NO: 11. In some embodiments, an antibody provided herein comprises a V_{H} sequence of SEQ ID NO: 12. In some embodiments, an antibody provided herein comprises a V_{H} sequence of SEQ ID NO: 13. In some embodiments, an antibody provided herein comprises a V_{H} sequence of SEQ ID NO: 14. In some embodiments, an antibody provided herein comprises a V_{H} sequence of SEQ ID NO: 15. In some embodiments, an antibody provided herein comprises a V_{H} sequence of SEQ ID NO: 16. In some embodiments, an antibody provided herein comprises a V_{H} sequence of SEQ ID NO: 17. In some embodiments, an antibody provided herein comprises a V_{H} sequence of SEQ ID NO: 18. In some embodiments, an antibody provided herein comprises a V_{H} sequence of SEQ ID NO: 19. In some embodiments, an antibody provided herein comprises a V_{H} sequence of SEQ ID NO: 20.

In some embodiments, a fibrin antigen binding region provided herein comprises a V_{H} sequence having at least about 50%, 60%, 70%, 80%, 90%, 95%, or 99% identity to an illustrative V_{H} sequence provided in SEQ ID NOs: 7, 8, 9, 10, 11, 12, 13, 14, 15, 16, 17, 18, 19, and 20. In some embodiments, an antibody provided herein comprises a V_{H} sequence provided in SEQ ID NOs: 7, 8, 9, 10, 11, 12, 13, 14, 15, 16, 17, 18, 19, and 20, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### V_{L} Domains

In some embodiments, a fibrin antigen binding region provided herein comprises a VL sequence selected from SEQ ID NO: 21.

In some embodiments, an antibody provided herein comprises a VL sequence having at least about 50%, 60%, 70%, 80%, 90%, 95%, or 99% identity to an illustrative VL sequence provided in SEQ ID NO: 21. In some embodiments, an antibody provided herein comprises a VL sequence provided in SEQ ID NO: 21 with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

In some embodiments, a fibrin antigen binding region provided herein comprises a V_{H} sequence selected from SEQ ID NOs: 7, 8, 9, 10, 11, 12, 13, 14, 15, 16, 17, 18, 19, and 20; and a V_{L} sequence selected from SEQ ID NO: 21.

In some embodiments, an antibody provided herein comprises a V_{H} sequence of SEQ ID NO: 7 and a V_{L} sequence of SEQ ID NO: 21. In some embodiments, an antibody provided herein comprises a V_{H} sequence of SEQ ID NO: 8 and a V_{L} sequence of SEQ ID NO: 21. In some embodiments, an antibody provided herein comprises a V_{H} sequence of SEQ ID NO: 9 and a V_{L} sequence of SEQ ID NO: 21. In some embodiments, an antibody provided herein comprises a V_{H} sequence of SEQ ID NO: 10 and a V_{L} sequence of SEQ ID NO: 21. In some embodiments, an antibody provided herein comprises a V_{H} sequence of SEQ ID NO: 11 and a V_{L} sequence of SEQ ID NO: 21. In some embodiments, an antibody provided herein comprises a V_{H} sequence of SEQ ID NO: 12 and a V_{L} sequence of SEQ ID NO: 21. In some embodiments, an antibody provided herein comprises a V_{H} sequence of SEQ ID NO: 13 and a V_{L} sequence of SEQ ID NO: 21. In some embodiments, an antibody provided herein comprises a V_{H} sequence of SEQ ID NO: 14 and a V_{L} sequence of SEQ ID NO: 21. In some embodiments, an antibody provided herein comprises a V_{H} sequence of SEQ ID NO: 15 and a V_{L} sequence of SEQ ID NO: 21. In some embodiments, an antibody provided herein comprises a V_{H} sequence of SEQ ID NO: 16 and a V_{L} sequence of SEQ ID NO: 21. In some embodiments, an antibody provided herein comprises a V_{H} sequence of SEQ ID NO: 17 and a V_{L} sequence of SEQ ID NO: 21. In some embodiments, an antibody provided herein comprises a V_{H} sequence of SEQ ID NO: 18 and a V_{L} sequence of SEQ ID NO: 21. In some embodiments, an antibody provided herein comprises a V_{H} sequence of SEQ ID NO: 19 and a V_{L} sequence of SEQ ID NO: 21. In some embodiments, an antibody provided herein comprises a V_{H} sequence of SEQ ID NO: 20 and a V_{L} sequence of SEQ ID NO: 21.

In certain aspects, any of SEQ ID NOs: 7, 8, 9, 10, 11, 12, 13, 14, 15, 16, 17, 18, 19, and 20 can be combined with any of SEQ ID NO: 21.

In some embodiments, a fibrin antigen binding region provided herein comprises a V_{H} sequence having at least about 50%, 60%, 70%, 80%, 90%, 95%, or 99% identity to an illustrative V_{H} sequence provided in SEQ ID NOs: 7, 8, 9, 10, 11, 12, 13, 14, 15, 16, 17, 18, 19, and 20; and a V_{L} sequence having at least about 50%, 60%, 70%, 80%, 90%, 95%, or 99% identity to an illustrative V_{L} sequence provided in SEQ ID NO: 21. In some embodiments, an antibody provided herein comprises a VH sequence provided in SEQ ID NOs: 7, 8, 9, 10, 11, 12, 13, 14, 15, 16, 17, 18, 19, and 20, with up to 1, 2, 3, 4, 5, 6, 7, 8, 9, 10, 11, 12, 13, 14, 15, 16, 17, 18, 19, 20, 21, 22, 23, 24, or 25 amino acid substitutions, and a VL sequence provided in SEQ ID NO: 21, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### CDRs

In some embodiments, a fibrin antigen binding region provided herein comprises one to three CDRs of a V_{H} domain selected from SEQ ID NOs: 7, 8, 9, 10, 11, 12, 13, 14, 15, 16, 17, 18, 19, and 20. In some embodiments, an antibody provided herein comprises two to three CDRs of a V_{H} domain selected from SEQ ID NOs: 7, 8, 9, 10, 11, 12, 13, 14, 15, 16, 17, 18, 19, and 20. In some embodiments, an antibody provided herein comprises three CDRs of a VH domain selected from SEQ ID NOs: 37, 8, 9, 10, 11, 12, 13, 14, 15, 16, 17, 18, 19, and 20. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 7, 8, 9, 10, 11, 12, 13, 14, 15, 16, 17, 18, 19, and 20. In some embodiments, the CDR-H1 is a CDR-H1 of a VH domain selected from SEQ ID NOs: 7, 8, 9, 10, 11, 12, 13, 14, 15, 16, 17, 18, 19, and 20, with up to 1, 2, 3, 4, or 5 amino acid substitutions. In some embodiments, the CDR-H2 is a CDR-H2 of a VH domain selected from SEQ ID NOs: 7, 8, 9, 10, 11, 12, 13, 14, 15, 16, 17, 18, 19, and 20, with up to 1, 2, 3, 4, 5, 6, 7, or 8 amino acid substitutions. In some embodiments, the CDR-H3 is a CDR-H3 of a VH domain selected from SEQ ID NOs: 7, 8, 9, 10, 11, 12, 13, 14, 15, 16, 17, 18, 19, and 20, with up to 1, 2, 3, 4, 5, 6, 7, or 8 amino acid substitutions. In some aspects, the amino acid substitutions are conservative amino acid substitutions. In some embodiments, the a fibrin antigen binding region described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises one to three CDRs of a VL domain of SEQ ID NO: 21. In some embodiments, an antibody provided herein comprises two to three CDRs of a VL domain of SEQ ID NO: 21. In some embodiments, an antibody provided herein comprises three CDRs of a VL domain of SEQ ID NO: 21. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-L1, CDR-L2, or CDR-L3 of SEQ ID NO: 21. In some embodiments, the CDR-L1 is a CDR-L1 of a VL domain of SEQ ID NO: 21, with up to 1, 2, 3, 4, or 5 amino acid substitutions. In some embodiments, the CDR-L2 is a CDR-L2 of a VL domain of SEQ ID NO: 21, with up to 1, 2, 3, 4, 5, 6, 7, or 8 amino acid substitutions. In some embodiments, the CDR-L3 is a CDR-L3 of a VL domain of SEQ ID NO: 21, with up to 1, 2, 3, 4, 5, 6, 7, or 8 amino acid substitutions. In some aspects, the amino acid substitutions are conservative amino acid substitutions. In some embodiments, a fibrin antigen binding region described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises one to three CDRs of a VH domain selected from SEQ ID NOs: 7, 8, 9, 10, 11, 12, 13, 14, 15, 16, 17, 18, 19, and 20 and one to three CDRs of a VL domain of SEQ ID NO: 21. In some embodiments, an antibody provided herein comprises two to three CDRs of a VH domain selected from SEQ ID NOs: 7, 8, 9, 10, 11, 12, 13, 14, 15, 16, 17, 18, 19, and 20 and two to three CDRs of a VL domain of SEQ ID NO: 21. In some embodiments, an antibody provided herein comprises three CDRs of a VH domain selected from SEQ ID NOs: 7, 8, 9, 10, 11, 12, 13, 14, 15, 16, 17, 18, 19, and 20 and three CDRs of a VL domain of SEQ ID NO: 21. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, a fibrin antigen binding region provided herein comprises a CDR-H3 selected of SEQ ID NOs: 24, 25, 26, 27, 28, 29, and 30. In some aspects, the CDR-H3 has at least about 50%, 75%, 80%, 85%, 90%, or 95% identity with a CDR-H3 of SEQ ID NOs: 24, 25, 26, 27, 28, 29, and 30. In some embodiments, the CDR-H3 is a CDR-H3 selected of SEQ ID NOs: 24, 25, 26, 27, 28, 29, and 30, with up to 1, 2, 3, 4, 5, 6, 7, or 8 amino acid substitutions. In some aspects, the amino acid substitutions are conservative amino acid substitutions. In some embodiments, a fibrin antigen binding region described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 24. In some aspects, the CDR-H3 has at least about 50%, 75%, 80%, 85%, 90%, or 95% identity with a CDR-H3 of SEQ ID NO: 24. In some embodiments, the CDR-H3 is a CDR-H3 selected of SEQ ID NO: 24, with up to 1, 2, 3, 4, 5, 6, 7, or 8 amino acid substitutions. In some aspects, the amino acid substitutions are conservative amino acid substitutions. In some embodiments, the fibrin antigen binding region described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 25. In some aspects, the CDR-H3 has at least about 50%, 75%, 80%, 85%, 90%, or 95% identity with a CDR-H3 of SEQ ID NO: 25. In some embodiments, the CDR-H3 is a CDR-H3 selected of SEQ ID NO: 25, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 26. In some aspects, the CDR-H3 has at least about 50%, 75%, 80%, 85%, 90%, or 95% identity with a CDR-H3 of SEQ ID NO: 26. In some embodiments, the CDR-H3 is a CDR-H3 selected of SEQ ID NO: 26, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 27. In some aspects, the CDR-H3 has at least about 50%, 75%, 80%, 85%, 90%, or 95% identity with a CDR-H3 of SEQ ID NO: 27. In some embodiments, the CDR-H3 is a CDR-H3 selected of SEQ ID NO: 27, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 28. In some aspects, the CDR-H3 has at least about 50%, 75%, 80%, 85%, 90%, or 95% identity with a CDR-H3 of SEQ ID NO: 28. In some embodiments, the CDR-H3 is a CDR-H3 selected of SEQ ID NO: 28, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 29. In some aspects, the CDR-H3 has at least about 50%, 75%, 80%, 85%, 90%, or 95% identity with a CDR-H3 of SEQ ID NO: 29. In some embodiments, the CDR-H3 is a CDR-H3 selected of SEQ ID NO: 29, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 30. In some aspects, the CDR-H3 has at least about 50%, 75%, 80%, 85%, 90%, or 95% identity with a CDR-H3 of SEQ ID NO: 30. In some embodiments, the CDR-H3 is a CDR-H3 selected of SEQ ID NO: 30, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 selected of SEQ ID NO: 3. In some aspects, the CDR-H3 has at least about 50%, 75%, 80%, 85%, 90%, or 95% identity with a CDR-H3 of SEQ ID NO: 3. In some embodiments, the CDR-H3 is a CDR-H3 selected of SEQ ID NO: 3, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H1 of SEQ ID NO: 1. In some aspects, the CDR-H1 has at least about 50%, 75%, 80%, 85%, 90%, or 95% identity with a CDR-H1 of SEQ ID NO: 1. In some embodiments, the CDR-H1 is a CDR-H1 of SEQ ID NO: 1, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H2 selected of SEQ ID NO: 2. In some aspects, the CDR-H2 has at least about 50%, 75%, 80%, 85%, 90%, or 95% identity with a CDR-H2 of SEQ ID NO: 2. In some embodiments, the CDR-H2 is a CDR-H2 selected of SEQ ID NO: 2, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 24 and a CDR-H2 of SEQ ID NO: 2. In some embodiments, an antibody provided herein comprises a CDR-H3 of SEQ ID NO: 25, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 24, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 24, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 25 and a CDR-H2 of SEQ ID NO: 2. In some embodiments, an antibody provided herein comprises a CDR-H3 of SEQ ID NO: 25, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 25, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 25, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 26 and a CDR-H2 of SEQ ID NO: 2. In some embodiments, an antibody provided herein comprises a CDR-H3 of SEQ ID NO: 26, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 26, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 26, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 27 and a CDR-H2 of SEQ ID NO: 2. In some embodiments, an antibody provided herein comprises a CDR-H3 of SEQ ID NO: 27, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 27, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 27, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an fibrin antigen binding region provided herein comprises a CDR-H3 of SEQ ID NO: 28 and a CDR-H2 of SEQ ID NO: 2. In some embodiments, an antibody provided herein comprises a CDR-H3 of SEQ ID NO: 28, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 28, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 28, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 29 and a CDR-H2 of SEQ ID NO: 2. In some embodiments, an antibody provided herein comprises a CDR-H3 of SEQ ID NO: 29, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 29, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 29, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 30 and a CDR-H2 of SEQ ID NO: 2. In some embodiments, an antibody provided herein comprises a CDR-H3 of SEQ ID NO: 30, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 30, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 30, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-L3 of SEQ ID NO: 6. In some aspects, the CDR-L3 has at least about 50%, 75%, 80%, 85%, 90%, or 95% identity with a CDR-L3 of SEQ ID NO: 6. In some embodiments, the CDR-L3 is a CDR-L3 of SEQ ID NO: 6, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-L2 of SEQ ID NO: 5. In some aspects, the CDR-L2 has at least about 50%, 75%, 80%, 85%, 90%, or 95% identity with a CDR-L2 of SEQ ID NO: 5. In some embodiments, the CDR-L2 is a CDR-L2 of SEQ ID NO: 5, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-L1 of SEQ ID NO: 4. In some aspects, the CDR-L1 has at least about 50%, 75%, 80%, 85%, 90%, or 95% identity with a CDR-L1 of SEQ ID NO: 4. In some embodiments, the CDR-L1 is a CDR-L1 of SEQ ID NO: 4, with up to 1, 2, 3, 4, 5, 6, 7, or 8 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-L3 of SEQ ID NO: 6 and a CDR-L2 of SEQ ID NO: 5. In some embodiments, an antibody provided herein comprises a CDR-L3 of SEQ ID NO: 6, a CDR-L2 of SEQ ID NO: 5, and a CDR-L1 of SEQ ID NO: 4. In some embodiments, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding regions described herein are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 24, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 24, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 24, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 25, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 25, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-Lt of SEQ ID NO: 4. In some embodiments, the CDR-H3 is a CDR-H3 of SEQ ID NO: 25, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding region described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 26, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 26, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 26, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 27, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 27, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 27, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 28, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 28, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 28, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 29, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 29, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 29, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding region described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H3 of SEQ ID NO: 30, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 30, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 30, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the fibrin antigen binding regions described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, a fibrin antigen binding region provided herein comprises a CDR-H1 of SEQ ID NO: 1, a CDR-H2 of SEQ ID NO: 2, a CDR-H3 of SEQ ID NO: 24, a CDR-L1 of SEQ ID NO: 4, a CDR-L2 of SEQ ID NO: 5, and a CDR-L3 of SEQ ID NO: 6.

In some embodiments, a fibrin antigen binding region provided herein comprises a CDR-H1 of SEQ ID NO: 1, a CDR-H2 of SEQ ID NO: 2, a CDR-H3 of SEQ ID NO: 25, a CDR-L1 of SEQ ID NO: 4, a CDR-L2 of SEQ ID NO: 5, and a CDR-L3 of SEQ ID NO: 6.

In some embodiments, a fibrin antigen binding region provided herein comprises a CDR-H1 of SEQ ID NO: 1, a CDR-H2 of SEQ ID NO: 2, a CDR-H3 of SEQ ID NO: 26, a CDR-L1 of SEQ ID NO: 4, a CDR-L2 of SEQ ID NO: 5, and a CDR-L3 of SEQ ID NO: 6.

In some embodiments, a fibrin antigen binding region provided herein comprises a CDR-H1 of SEQ ID NO: 1, a CDR-H2 of SEQ ID NO: 2, a CDR-H3 of SEQ ID NO: 27, a CDR-L1 of SEQ ID NO: 4, a CDR-L2 of SEQ ID NO: 5, and a CDR-L3 of SEQ ID NO: 6.

In some embodiments, a fibrin antigen binding region provided herein comprises a CDR-H1 of SEQ ID NO: 1, a CDR-H2 of SEQ ID NO: 2, a CDR-H3 of SEQ ID NO: 28, a CDR-L1 of SEQ ID NO: 4, a CDR-L2 of SEQ ID NO: 5, and a CDR-L3 of SEQ ID NO: 6.

In some embodiments, a fibrin antigen binding region provided herein comprises a CDR-H1 of SEQ ID NO: 1, a CDR-H2 of SEQ ID NO: 2, a CDR-H3 of SEQ ID NO: 29, a CDR-L1 of SEQ ID NO: 4, a CDR-L2 of SEQ ID NO: 5, and a CDR-L3 of SEQ ID NO: 6.

In some embodiments, a fibrin antigen binding region provided herein comprises a CDR-H1 of SEQ ID NO: 1, a CDR-H2 of SEQ ID NO: 2, a CDR-H3 of SEQ ID NO: 30, a CDR-L1 of SEQ ID NO: 4, a CDR-L2 of SEQ ID NO: 5, and a CDR-L3 of SEQ ID NO: 6.

### Epitopes

In certain embodiments, described herein are fibrin antigen binding regions that binds human fibrin or fibrinogen γC domain, wherein the fibrin antigen binding region binds human fibrin at any one of amino acid residues Lys 411, Ile 412, Ile 413, Phe 415, Asn 416, Arg 417, Leu 418, Thr 419, Ile 420, and Gly 421. In certain embodiments, the fibrin antigen binding region binds human fibrin at at least two, three, four, five, six, seven, eight, nine, or all ten of amino acid residues Lys 411, Ile 412, Ile 413, Phe 415, Asn 416, Arg 417, Leu 418, Thr 419, Ile 420, and Gly 421. In certain embodiments, the fibrin antigen binding region binds human fibrin at amino acid residues Lys 411, Ile 412, Ile 413, Phe 415, Asn 416, Arg 417, Leu 418, Thr 419, Ile 420, and Gly 421. In certain embodiments the amino acid residue of the human fibrin or fibrinogen γC domain epitope bind the paratope of the antibody with a distance of less than 5 Angstroms or less, 4 Angstroms or less, 3 Angstroms or less, or 2 Angstroms or less.

### Paratopes

In certain embodiments, the fibrin antigen binding regions described herein comprise a VH region comprising a paratope that binds human fibrin or fibrinogen γC domain, wherein the paratope comprises any one of amino acid residues Ser 31, Tyr 32, Trp 33, His 35, Trp 47, Leu 50, Asp 52, Asp 54, Tyr 56, Ala 93, Ser 94, Ser 95, Lys 96 or Asp 96, Pro 97 or Ala 97, Gly 101, Gly 102, and Trp 103. In certain embodiments, the fibrin antigen binding region comprises a VH region comprising a paratope that comprises at least two, three, four, five, six, seven, eight, nine, ten, eleven, twelve, thirteen, fourteen, fifteen, sixteen, or all seventeen of amino acid residues Ser 31, Tyr 32, Trp 33, His 35, Trp 47, Leu 50, Asp 52, Asp 54, Tyr 56, Ala 93, Ser 94, Ser 95, Lys 96 or Asp 96, Pro 97 or Ala 97, Gly 101, Gly 102, and Trp 103. In certain embodiments, the antibody comprises a VH region comprising a paratope that comprises amino acid residues Ser 31, Trp 33, His 35, Asp 52, Asp 54, Tyr 56, Ser 94, Gly 101, Gly 102, and Trp 103. In certain embodiments, the fibrin antigen binding region comprises a VH region comprising a paratope that comprises amino acid residues Ser 31, Trp 33, His 35, Asp 52, Asp 54, Tyr 56, Ala 93, Ser 94, Lys 96, Pro 97, Gly 101, Gly 102, and Trp 103. In certain embodiments, the fibrin antigen binding region comprises a VH region comprising a paratope that comprises amino acid residues Ser 31, Tyr 32, Trp 33, His 35, Trp 47, Asp 52, Asp 54, Tyr 56, Ser 94, Ser 95, Asp 96, Ala 97, Gly 101, Gly 102, and Trp 103.

In certain embodiments, the fibrin antigen binding region comprises a VL region comprising a paratope that comprises any one of amino acid residues His 27, Tyr 32, Tyr 36, Leu 46, Tyr 49, Gln 50, Ala 91 or Asn 91, Leu 92, Leu 94, and Leu 96. In certain embodiments, the antibody comprises a VL region comprising a paratope that comprises at least two, three, four, five, six, seven, eight, nine or all ten amino acid residues His 27, Tyr 32, Tyr 36, Leu 46, Tyr 49, Gln 50, Ala 91 or Asn 91, Leu 92, Leu 94, and Leu 96. In certain embodiments, the antibody comprises a VL region comprising a paratope that comprises the amino acid residues His 27, Tyr 32, Tyr 36, Leu 46, Gln 50, Leu 92, Leu 94, and Leu 96. In certain embodiments, the antibody comprises a VL region comprising a paratope that comprises the amino acid residues His 27, Tyr 32, Tyr 36, Leu 46, Gln 50, Asn 91, Leu 92, Leu 94, and Leu 96. In certain embodiments, the antibody comprises a VL region comprising a paratope that comprises the amino acid residues His 27, Tyr 32, Tyr 36, Leu 46, Tyr 49, Gln 50, Ala 91, Leu 92, Leu 94, and Leu 96.

In certain embodiments the paratope of the fibrin antigen binding region binds the amino acid residues of the human fibrin or fibrinogen γC domain epitope with a distance of less than 5 Angstroms or less, 4 Angstroms or less, 3 Angstroms or less, or 2 Angstroms or less.

### VEGF Antigen Binding Regions

In certain aspects, the multi-specific antigen binding proteins described herein comprise at least one antigen binding region that binds VEGF. In certain aspects, the antigen binding region that binds VEGF, binds one or more of VEGF-A, VEGF-B, VEGF-C and VEGF-D. In certain embodiments, the antigen binding region that binds VEGF comprises a VH amino acid sequence that is 90% or more, 95% or more, or 99% or more identical to the VH amino acid sequence set forth in SEQ ID NO: 269. In certain embodiments, the VEGF antigen binding region comprises a VL amino acid sequence that is 90% or more, 95% or more, or 99% or more identical to the VL amino acid sequence set forth in SEQ ID NO: 268. In certain embodiments, the VEGF antigen binding region binds an epitope of VEGF-A comprising at least one of amino acids 82-91 of SEQ ID NO. 265. In certain embodiments, the VEGF antigen binding region binds an epitope of VEGF-A comprising amino acids 82-91 of SEQ ID NO. 265. In certain embodiments, the VEGF antigen binding region comprises a sequence that is 90% or more, 95% or more, or 99% or more identical to the amino acid sequence set forth in SEQ ID NOs: 266 or 267.

In certain embodiments, the VEGF antigen binding region is a single domain antibody, a diabody, an scFv, an scFv dimer, a BsFv, a dsFv, a (dsFv)2, a dsFv-dsFv′, an Fv fragment, a Fab, a Fab′, a F(ab′)2, a ds diabody, a minibody, a nanobody, a domain antibody, or a bivalent domain antibody. In certain embodiments, the VEGF antigen binding region is not an antibody. In certain embodiments, the VEGF antigen binding region is an antibody that does not comprise and Fc region. In certain embodiments, the VEGF antigen binding region comprises an Fc region of the class IgA, IgD, IgE, IgG, or IgM. In certain embodiments, the VEGF antigen binding region comprises one or more modifications known in the art to stabilize the Fc region and improve circulation half-life in vivo. In certain embodiments, the VEGF antigen binding region comprises one or more modifications to promote selective binding of Fc-gamma receptors and/or C1q.

In certain embodiments, the multi-specific antigen binding proteins described herein comprise a plurality of VEGF antigen binding region. In certain embodiments, the multi-specific antigen binding proteins comprise 2, 3, 4, 5, or more VEGF antigen binding regions. In certain embodiments, the multi-specific antigen binding proteins comprise more than one distinct VEGF antigen binding region.

### Fc Region

The structures of the Fc regions of various immunoglobulins, and the glycosylation sites contained therein, are known in the art. See Schroeder and Cavacini, J. Allergy Clin. Immunol., 2010, 125:541-52, incorporated by reference in its entirety. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG₁, IgG₂, IgG₃, IgG₄, IgA₁, and IgA₂.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fc gamma receptors. In some aspects, the Fc comprises one or more modifications to promote selective binding of Fc-gamma receptors.

Exemplary mutations that alter the binding of FcRs to the Fc are listed below:
S298A/E333A/K334A, S298A/E333A/K334A/K326A (Lu Y, Vernes J M, Chiang N, et al. J Immunol Methods. 2011 Feb. 28; 365(1-2):132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13(6):R123);
F243L (Stewart R, Thom G, Levens M, et al. Protein Eng Des Sel. 2011 September; 24(9):671-8.), S298A/E333A/K334A (Shields R L, Namenuk A K, Hong K, et al. J Biol Chem. 2001 Mar. 2; 276(9):6591-604);
S239D/I332E/A330L, S239D/I332E (Lazar G A, Dang W, Karki S, et al. Proc Natl Acad Sci USA. 2006 Mar. 14; 103(11):4005-10);
S239D/S267E, S267E/L328F (Chu S Y, Vostiar I, Karki S, et al. Mol Immunol. 2008 September; 45(15):3926-33);
S239D/D265S/S298A/I332E, S239E/S298A/K326A/A327H, G237F/S298A/A330L/I332E, S239D/I332E/S298A, S239D/K326E/A330L/I332E/S298A, G236A/S239D/D270L/I332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, Oct. 2012) lists mutations on page 283.

In some embodiments an antibody described herein includes modifications to improve its ability to mediate effector function. Such modifications are known in the art and include afucosylation, or engineering of the affinity of the Fc towards an activating receptor, mainly FCGR3a for ADCC, and towards C1q for CDC. The following Table B summarizes various designs reported in the literature for effector function engineering.

Methods of producing antibodies with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 December; 20 (12):1607-18. Another approach to obtaining antibodies with lowered levels of fucosylation can be found in U.S. Pat. No. 8,409,572, which teaches selecting cell lines for antibody production for their ability to yield lower levels of fucosylation on antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

Thus, in one embodiment, an antibody described herein can include a dimeric Fc that comprises one or more amino acid modifications as noted in Table B that confer improved effector function. In another embodiment, the antibody can be afucosylated to improve effector function.

TABLE B
Table B. CH2 domains and effector function engineering

| Reference | Mutations | Effect |
|---|---|---|
| Lu, 2011, | Afucosylated | Increased ADCC |
| Ferrara 2011, | | |
| Mizushima 2011 | | |
| Lu, 2011 | S298A/E333A/K334A | Increased ADCC |
| Lu, 2011 | S298A/E333A/K334A/ | Increased ADCC |
| | K326A | |
| Stavenhagen, 2007 | F243L/R292P/Y300L/ | Increased ADCC |
| | V305I/P396L | |
| Nordstrom, 2011 | F243L/R292P/Y300L/ | Increased ADCC |
| | L235V/P396L | |
| Stewart, 2011 | F243L | Increased ADCC |
| Shields, 2001 | S298A/E333A/K334A | Increased ADCC |
| Lazar, 2006 | S239D/I332E/A330L | Increased ADCC |
| Lazar, 2006 | S239D/I332E | Increased ADCC |
| Bowles, 2006 | AME-D, not specified | Increased ADCC |
| | mutations | |
| Heider, 2011 | 37.1, mutations | Increased ADCC |
| | not disclosed | |
| Moore, 2010 | S267E/H268F/S324T | Increased CDC |

Fc modifications reducing FcgR and/or complement binding and/or effector function are known in the art. Recent publications describe strategies that have been used to engineer antibodies with reduced or silenced effector activity (see Strohl, W R (2009), Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, US Patent Publication No. 2011/0212087 (Strohl), International Patent Publication No. WO 2006/105338 (Xencor), US Patent Publication No. 2012/0225058 (Xencor), US Patent Publication No. 2012/0251531 (Genentech), and Strop et al ((2012) J. Mol. Biol. 420: 204-219) describe specific modifications to reduce FcgR or complement binding to the Fc.

Specific, non-limiting examples of known amino acid modifications to reduce FcgR or complement binding to the Fc include those identified in the following Table C:

TABLE C
Table C. Modifications to reduce FcgR
or complement binding to the Fc

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glyco |
| Medimune | L234F/L235E/P331S |
| Trubion | Hinge mutant, possibly C226S/P230S |

Methods of producing antibodies with little or no fucose on the Fc glycosylation site (Asn 297 EU numbering) without altering the amino acid sequence are well known in the art. The GlymaxX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 December; 20 (12):1607-18.) Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies.

Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety).

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al., Biotech. Bioeng., 2004, 87: 614-622; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which his incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of a fibrin antibody for a non-target molecule is less than about 50% of the affinity for fibrin. In some embodiments, the affinity of a fibrin antibody for a non-target molecule is less than about 40% of the affinity for fibrin. In some embodiments, the affinity of a fibrin antibody for a non-target molecule is less than about 30% of the affinity for fibrin. In some embodiments, the affinity of a fibrin antibody for a non-target molecule is less than about 20% of the affinity for fibrin. In some embodiments, the affinity of a fibrin antibody for a non-target molecule is less than about 10% of the affinity for fibrin. In some embodiments, the affinity of a fibrin antibody for a non-target molecule is less than about 1% of the affinity for fibrin. In some embodiments, the affinity of a fibrin antibody for a non-target molecule is less than about 0.1% of the affinity for fibrin.

When used herein in the context of two or more antibodies, the term “competes with” or “cross-competes with” indicates that the two or more antibodies compete for binding to an antigen (e.g., fibrin). In one exemplary assay, fibrin is coated on a surface and contacted with a first fibrin antibody, after which a second fibrin antibody is added. In another exemplary assay, a first fibrin antibody is coated on a surface and contacted with fibrin, and then a second fibrin antibody is added. If the presence of the first fibrin antibody reduces binding of the second fibrin antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the antibodies used in the competition assays based on the affinities of the antibodies for fibrin and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Antibodies identified by competition assay (competing antibody) include antibodies binding to the same epitope as the reference antibody and antibodies binding to an adjacent epitope sufficiently proximal to the epitope bound by the reference antibody for steric hindrance to occur. For example, a second, competing antibody can be identified that competes for binding to fibrin with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In some embodiments, an anti-fibrin antibody does not substantially bind myeloid cells present outside of cancer tissue. In some embodiments, an anti-fibrin antibody does not substantially bind stimulatory myeloid cells present in cancer tissue.

In some embodiments, an anti-fibrin antibody binds to residues γ377-395 of the fibrin or fibrinogen γC domain (SEQ ID NO: 31) of human fibrin. The binding epitope includes the residues within the numerical range (e.g., residues 377-395 of fibrin), the beginning residue of each range (e.g., residues 377-394 of human fibrin) and the end residue of each range (e.g., residues 378-395 of human fibrin), or any combination thereof.

In some embodiments, an antibody provided herein binds human Fibrin with a K_{D} of less than or equal to about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁶ M, as measured by Biacore assay. In some embodiments, the K_{D} of the antibody provided herein is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁶ M, as measured by Biacore assay. In some embodiments, an antibody provided herein binds human Fibrin with a K_{D} of less than or equal to about 1×10⁻⁵ M, 1×10⁻⁶ M, 1×10⁻⁷ M, 1×10⁻⁸ M, or 1×10⁻⁹ M.

In some embodiments, the antibody provided herein binds human fibrin with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁵ M, or less, as measured by Biacore assay. In some embodiments, the antibody provided herein binds human fibrin with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁵ M as measured by Biacore assay. In some embodiments, the antibody provided herein binds human fibrin with a K_{d} of less than or equal to about 10, 9.56, 9.5, 9.0, 8.88, 8.84, 8.5, 8, 7.5, 7.32, 7, 6.5, 6, 5.5, 5, 4.5, 4, 3.5, 3, 2.5, 2, 1.5, or 1×10⁻⁴ (1/s), or less, as measured by Biacore assay. In some embodiments, the antibody provided herein binds human fibrin with a K_{d} between 7-10, 7-8, 8-9, 9-10, 7-7.5, 7.5-8, 8-8.5, 8.5-9, 9-9.5, or 9.5-10×10⁻⁴ (1/s) as measured by Biacore assay. In some embodiments, the antibody provided herein binds human fibrin with a Kₐ of greater than or equal to about 4, 4.1, 4.2, 4.3, 4.4, 4.5, 4.6, 4.7, 4.8, 4.9, 45, 5.1, 5.2, 5.3, 5.4, 5.5, 5.6, 5.7, 5.8, 5.9, 6, 7, 8, 9, or 10×10⁵ (1/Ms), or more, as measured by Biacore assay. In some embodiments, the antibody provided herein binds human FIBRIN with a Kₐ between 4-7, 4-4.5, 4.5-5, 5-5.5, 5.5-6, 6-6.5, or 6.5-7, 7-8, 8-9, or 9-10×10⁵ (1/Ms) as measured by Biacore assay.

### Function

“Effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP). In some embodiments, the effector function of the fibrin antibody described herein is antagonism and blocks Mac-1 receptor binding to fibrin.

### Pharmaceutical Compositions

The present application provides compositions comprising the antibodies including pharmaceutical compositions comprising any one or more of the antibodies described herein with one or more pharmaceutically acceptable excipients. In some embodiments the composition is sterile. The pharmaceutical compositions generally comprise an effective amount of an antibody.

These compositions can comprise, in addition to one or more of the antibodies disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

The anti-fibrin antibody that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Methods

### Methods of Preparation

Antibodies described herein can be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such nucleic acid are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, or human embryonic kidney (HEK) cell, or lymphoid cell (e.g., Y0, NS0, Sp20 cell). In one embodiment, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

For recombinant production of the antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When a heteromultimer or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the heteromultimer or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” heteromultimer produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp20, CV-1, VERO-76, HeLa, HepG2, Per.C6, or BHK.

For example, antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the antibodies described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

In some embodiments, is the method of producing an antibody in stable mammalian cells as described herein wherein the expression product of the at least one stable mammalian cell comprises a larger percentage of the desired glycosylated antibody as compared to the monomeric heavy or light chain polypeptides, or other antibodies.

In some embodiments, is the method of producing a glycosylated antibody in stable mammalian cells described herein, said method comprising identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting fibrin and VEGF with an multi-specific antigen binding described herein which results in inhibition of microglial adhesion to the fibrin or fibrinogen γC domain and neutralization of VEGF.

In an aspect, the present application provides methods of using the multi-specific antigen binding proteins described herein for treatment of a pathology associated with Mac-1 binding to fibrin or Mac-1 binding with fibrinogen, the method comprising administering to a mammalian subject a therapeutically effective amount of a multi-specific antigen binding protein or a pharmaceutical composition comprising a multi-specific antigen binding protein described herein.

In certain aspects, described herein are methods of inhibiting microglia activation, the method comprising administering to a mammalian subject a therapeutically effective amount a multi-specific antigen binding protein or a pharmaceutical composition comprising a multi-specific antigen binding protein described herein.

In certain aspects, described herein are methods of treating or preventing a disorder or condition of the eye comprising administering to a mammalian subject a therapeutically effective amount of a multi-specific antigen binding protein or a pharmaceutical composition comprising a multi-specific antigen binding protein described herein. In certain embodiments, the disorder or condition of the eye is wet age-related macular degeneration (“WAMD”), age-related macular degeneration (“AMD”), diabetic retinopathy, diabetic macular edema (DME), central retinal vein occlusion (RVO), pathologic myopia, polypoidal choroidal vasculopathy, retinitis pigmentosa, glaucoma, uveitis, or retinal detachment.

### Methods of Administration

In some embodiments, the methods provided herein are useful for the treatment of a disorder or condition of the eye in an individual. In an embodiment, the individual is a human.

In some embodiments, the multi-specific antigen binding protein is administered intravenously, intramuscularly, subcutaneously, topically, orally, transdermally, intraperitoneally, intraorbitally, intravitreally, by implantation, by inhalation, intrathecally, intraventricularly, or intranasally. An effective amount of multi-specific antigen binding protein may be administered for the treatment of a disorder or condition of the eye. The appropriate dosage of the multi-specific antigen binding protein may be determined based on the type of eye condition to be treated, the type of the multi-specific antigen binding protein, the severity and course of the eye condition, the clinical condition of the individual, the individual's clinical history and response to the treatment, and the discretion of the attending physician.

In some embodiments, the multi-specific antigen binding protein provided herein is administered with at least one additional therapeutic agent. Any suitable additional therapeutic or immunotherapeutic agent may be administered with an antibody provided herein. Additional therapeutic agents include agents that are used to treat or prevent a disorder or condition of the eye selected from the group consisting of: wet age-related macular degeneration (“WAMD”), age-related macular degeneration (“AMD”), diabetic retinopathy, diabetic macular edema (DME), central retinal vein occlusion (RVO), pathologic myopia, polypoidal choroidal vasculopathy, retinitis pigmentosa, glaucoma, uveitis, or retinal detachment.

The additional therapeutic agent can be administered by any suitable means. In some embodiments, the multi-specific antigen binding protein provided herein and the additional therapeutic agent are included in the same pharmaceutical composition. In some embodiments, the multi-specific antigen binding protein provided herein and the additional therapeutic agent are included in different pharmaceutical compositions.

In embodiments where the multi-specific antigen binding protein provided herein and the additional therapeutic agent are included in different pharmaceutical compositions, administration of the multi-specific antigen binding protein can occur prior to, simultaneously, and/or following, administration of the additional therapeutic agent. In some embodiments, administration of the multi-specific antigen binding protein provided herein and the additional therapeutic agent occur within about one month of each other. In some embodiments, administration of the multi-specific antigen binding protein provided herein and the additional therapeutic agent occur within about one week of each other. In some embodiments, administration of the multi-specific antigen binding protein provided herein and the additional therapeutic agent occur within about one day of each other. In some embodiments, administration of the multi-specific antigen binding protein provided herein and the additional therapeutic agent occur within about twelve hours of each other. In some embodiments, administration of the multi-specific antigen binding protein provided herein and the additional therapeutic agent occur within about one hour of each other.

### Kits and Articles of Manufacture

The present application provides kits comprising any one or more of the multi-specific antigen binding protein described herein. In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the multi-specific antigen binding protein described herein, with one or more pharmaceutically acceptable excipients.

The present application also provides articles of manufacture comprising any one of the multi-specific antigen binding proteins or kits described herein. Examples of an article of manufacture include vials (including sealed vials).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Materials and Methods

Antigens were biotinylated using the EZ-Link Sulfo-NHS-Biotinylation Kit from Pierce. Goat F(ab′)₂ anti-human kappa-FITC (LC-FITC), ExtrAvidin-PE (EA-PE), and Streptavidin-AF633 (SA-633) were obtained from Southern Biotech, Sigma, and Molecular Probes, respectively. Goat anti-human IgG-PE (Human-PE) was obtained from Southern Biotech. Ami-Mouse APC was obtained from Jackson ImmunoResearch.

### Example 1: Anti-Fibrin P2 Treatment Decreases Inflammation in Uveitis Model

Experimental autoimmune uveitis (EAU) is an organ-specific autoimmune disease that targets the neural retina. This autoimmune response is induced when animals are immunized with retinal antigens (interphotoreceptor retinoid-binding protein (IRBP)], in this case). In order to confirm therapeutic role of anti-fibrin treatment in inflammatory eye conditions or diseases, the efficacy of the anti-fibrin affinity matured antibodies were tested in a rat EAU model after intravitreal administration of the anti-fibrin antibodies.

In this study, 52 Lewis rats were divided into six groups, namely PBS (Group 1), Isotype control (Group 2), ABI-60143 low dose (Group 3), ABI-60143 high dose (Group 4), FTY-720 positive control (Group 5), and Naïve (Group 6). Animals from all groups, except Group 6, were immunized with an emulsion of IRBP in Complete Freund's Adjuvant (CFA) on Day 0. Similarly, animals from Group 1-4 received a single intravitreal injection of sponsors test article once on Day 0. Animals in Group 5 received once daily oral administration of positive control FTY-720. After a period of 8-10 days, immunized animals developed uveitis in each eye. Clinical evaluations were performed for all the animals at baseline, day 4, 7, 11, and 14 to follow the extent of diseases developed. Clinical observations were performed as follows:
Frequency: Once on each study day.
Procedure: Groups were randomized ahead of evaluations to keep the examiner masked. Animals were observed under a dissection microscope and scored on a scale of 0-4 based on their anterior clinical disease. Photographs of the anterior chamber were taken at the time of clinical evaluations.
Clinical observation scoring:
   0-0.5: No disease; eye is translucent. Some blood vessels in the iris may be dilated.
   1: Engorged blood vessels in iris; abnormal pupil contraction (or dilation).
   2: Slight haziness to the anterior chamber.
   3: Moderately opaque anterior chamber, but pupil still visible.
   4: Opaque anterior chamber and obscured pupil.

All animals were euthanized on Day 14 and immediately following euthanasia, whole eyes (OU) were collected, Upon verification of death, both eyes of each animal were carefully removed. One eye was collected for histological analysis, and the other eye was collected for cytokine analysis. Eyes for cytokine analysis were hemisected and retina was collected. Each eye was carefully orientated for optimal microscopic examination prior to wax embedding. Sections (5 μm) were cut and stained with hematoxylin and eosin for histological examination and scoring according to the following scale summarized below and as described by Caspi, et al. (2012). Histological analysis was masked to the examiner. Clinical Scoring/Uveitis grading was determined as follows:
0: No disease, normal retinal architecture.
0.5: Trace. <¼ Mild inflammatory cell infiltration of the retina with or without photoreceptor damage.
1: ≥¼ Mild inflammation and/or photoreceptor outer segment damage.
2: ≥¼ Mild to moderate inflammation and/or lesion extending to the outer nuclear layer.
3: ≥¼ Moderate to marked inflammation and or lesion extending to the inner nuclear layer.
4: ≥¼ Severe inflammation and/or full-thickness retinal damage.

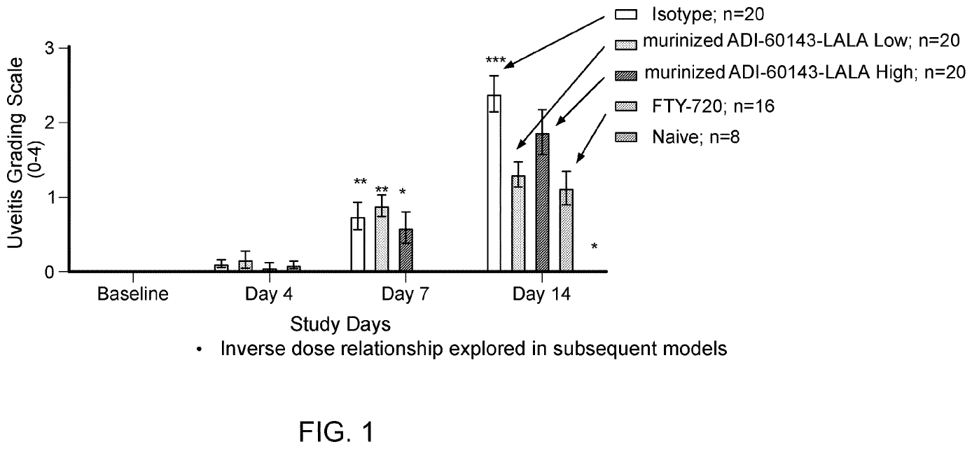

As shown in FIG. 1, rats administered a low or high dose of the murinized ADI-60143—LALA Fc-stabilized antibody clone exhibited a significantly reduced clinical uveitis score on Day 14 of the study. These results confirm that the affinity matured anti-fibrin antibodies decreased inflammation in subjects with uveitis and are therapeutically effective in a pre-clinical model of eye conditions related to vascular defects of the eye, such as uveitis.

### Example 2: Laser-Induced Choroidal Neovascularization Study

Laser-induced choroidal neovascularization (LCNV) was generated in 6- to 8-week-old Brown Norway rats on experiment Day 0. Animals were anesthetized with an intraperitoneal (IP) injection of ketamine/xylazine, and topical administration of 1% tropicamide for pupillary dilation. Using a handheld cover slip as a contact lens and Gen Teal lubricating eye gel as a medium contacting the cover slip to the surface of the cornea, a Nidek GYC-500 green laser photocoagulator coupled to a Nidek SL-1800 slitlamp was used to create six lesions equidistant from the optic nerve head in the retinal mid periphery.

Laser parameters included: 532 nm wavelength, 100 μm spot size, 0.1 sec duration, and 120 mW. Anti-Fibrin antibodies disclosed herein were administered via intravitreal (IVIT) injection on the same day as laser. 6 days post-laser, vascular leakage was assessed via quantitative fluorescein angiography (qFA). At 7 days post-laser, animals were sacrificed for choroidal neovascularization (CNV) area analysis.

On experiment Day 1, treatments were administered bilaterally via IVIT injection according to Table 1 below.

TABLE 1

| | | qFA* | OCT* | CNV Area | Serum | Histology |
|---|---|---|---|---|---|---|
| Arm | Treatment | (n = eyes) | (n = eyes) | (n = rats) | (n = rats) | (n = eyes) |
| 1 | Vehicle (PBS) | 6 | 6 | 11 | 6 | 6 |
| 2 | Negative Control | 6 | 6 | 11 | 6 | 6 |
| | (Isotype Control) | | | | | |
| 3 | Therini Asset Dose 1 | 6 | 6 | 11 | 6 | 6 |
| 4 | Therini Asset Dose 2 | 6 | 6 | 11 | 6 | 6 |
| 5 | Positive Control (Eylea) | 6 | 6 | 11 | 6 | 6 |

Total eyes = 85 + 15% attrition = 98 eyes, 49 rats

As qFA and optical coherence tomography (OCT) are non-terminal (e.g., survival procedure) procedures, rats used for these readouts were also used for CNV area analysis, serum collection, and histology.

Animals were anesthetized with isoflurane and treated with topical proparacaine before injection to provide topical analgesia. IVIT injections of up to 2.0 μL volume were given using a short barrel, 2 μL syringe with a custom made, detachable 33-gauge needle (Hamilton Co.) under an operating microscope (Zeiss Microscopy). After injection, neomycin/polymyxin B/gramicidin ophthalmic drops were applied to prevent infection. Treatments were randomized between animals and housing cages.

At experiment Day 0, Day 3, and Day 7, images of lesion placement were captured by OCT using the Heidelberg Spectralis. Animals were anesthetized with an intraperitoneal (IP) injection of ketamine/xylazine. A-scans were used to identify the location of CNV lesions, and high-resolution Bscans were captured for each lesion and 3D deconvolution was performed to determine lesion volume. As OCT is a survival procedure, animals were also used for serum collection.

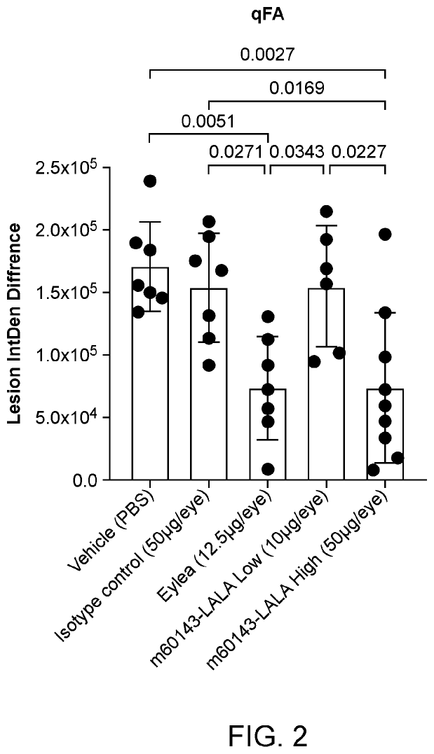

At experiment Day 6, vascular leakage was assessed via qFA (FIG. 2). Animals were anesthetized with an IP injection of ketamine/xylazine. Sodium fluorescein was administered via tail vein injection at a dose of 500 mg/kg. Fluorescent fundus images were captured for one eye of each animal with a Micron IV imaging system at four minutes post-fluorescein injection, and again two minutes later. ImageJ software was used by a masked observer to quantify the fluorescence intensity for one lesion per eye using the “integrated density” function. The difference in integrated density between the two times post-injection was recorded as a readout of vascular leakage. As qFA is a survival procedure, animals were used for CNV area.

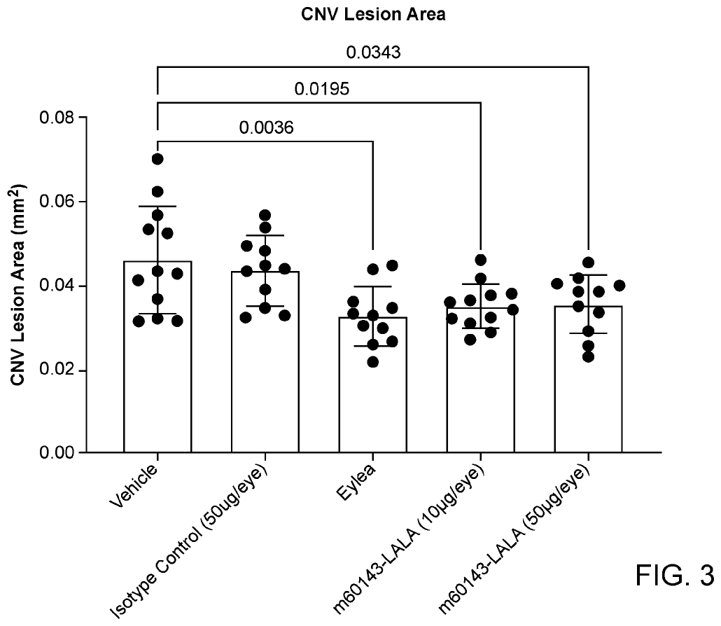

At experiment Day 7, animals were euthanized and their eyes enucleated. The extent of CNV at the Bruch's membrane rupture sites was measured by masked observers using computer-assisted image analysis of FITC-conjugated isolectin B4-stained choroidal flat-mounts. As the anti-fibrin antibody was administered to both eyes in the same animal, the areas for all lesions in both eyes of each animal was averaged and reported as a single data point (FIG. 3). Statistical analysis of area measurements were performed by ANOVA and a parametric post hoc test. Retinas were paraffin embedded and sectioned for further analysis.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

INFORMAL SEQUENCE LISTING

| | | SEQ ID |
|---|---|---|
| Description | Sequence | NO |
| 56666 | YTFTSYWIH | SEQ ID |
| CDR-H1 | | NO: 1 |
| 56666 | LIDPSDSYTNYNQKFRG | SEQ ID |
| CDR-H2 | | NO: 2 |
| 56666 | ASSDPTGG | SEQ ID |
| CDR-H3 | | NO: 3 |
| 56666 | RSSKSLLHSSGITYLS | SEQ ID |
| CDR-L1 | | NO: 4 |
| 56666 | QMSNLAS | SEQ ID |
| CDR-L2 | | NO: 5 |
| 56666 | AQNLELPLT | SEQ ID |
| CDR-L3 | | NO: 6 |
| 56666 | QVQLVQSGAEVKKPGASVKVSCKASGYTFTSYWIHWVRQAPGQGL | SEQ ID |
| VH | EWMGLIDPSDSYTNYNQKFRGRVTMTVDTSTSTAYMELSSLRSED | NO: 7 |
| | TAVYYCASSDPTGGWGQGTTVTVSS | |
| 56666 | ASTKGPSVFPLAPSSKSTSGGTAALGCLVKDYFPEPVTVSWNSGA | SEQ ID |
| CH | LTSGVHTFPAVLQSSGLYSLSSVVTVPSSSLGTQTYICNVNHKPS | NO: 8 |
| | NTKVDKKVEPKSCDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPG | |
| 56666 | RTVAAPSVFIFPPSDEQLKSGTASVVCLLNNFYPREAKVQWKVDN | SEQ ID |
| CL | ALQSGNSQESVTEQDSKDSTYSLSSTLTLSKADYEKHKVYACEVT | NO: 9 |
| | HQGLSSPVTKSFNRGEC | |
| 56666 | DIVMTQSPLSLPVTPGEPASISCRSSKSLLHSSGITYLSWYLQKP | SEQ ID |
| VL | GQSPQLLIYQMSNLASGVPDRFSGSGSGTDFTLKISRVEAEDVGV | NO: 10 |
| | YYCAQNLELPLTFGGGTKVEIK | |
| 56666 | QVQLVQSGAEVKKPGASVKVSCKASGYTFTSYWIHWVRQAPGQGL | SEQ ID |
| VH + CH | EWMGLIDPSDSYTNYNQKFRGRVTMTVDTSTSTAYMELSSLRSED | NO: 11 |
| | TAVYYCASSDPTGGWGQGTTVTVSSASTKGPSVFPLAPSSKSTSG | |
| | GTAALGCLVKDYFPEPVTVSWNSGALTSGVHTFPAVLQSSGLYSL | |
| | SSVVTVPSSSLGTQTYICNVNHKPSNTKVDKKVEPKSCDKTHTCP | |
| | PCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGK | |
| | EYKCKVSNKALPAPIEKTISKAKGQPREPQVYTLPPSRDELTKNQ | |
| | VSLTCLVKGFYPSDIAVEWESNGQPENNYKTTPPVLDSDGSFFLY | |
| | SKLTVDKSRWQQGNVFSCSVMHEALHNHYTQKSLSLSPG | |
| 56666 | AEDVGVYYCAQNLELPLTFGGGTKVEIKRTVAAPSVFIF | SEQ ID |
| VL + CL | PPSDEQLKSGTASVVCLLNNFYPREAKVQWKVDNALQSG | NO: 12 |
| | NSQESVTEQDSKDSTYSLSSTLTLSKADYEKHKVYACEV | |
| | THQGLSSPVTKSFNRGEC | |
| 61289 CDR- | YTFTSSWIH | SEQ ID |
| H1 | | NO: 13 |
| 61289 CDR- | LIDPSDSYTNYNQKFRG | SEQ ID |
| H2 | | NO: 14 |
| 61289 CDR- | ASSDPHGG | SEQ ID |
| H3 | | NO: 15 |
| 61289 CDR- | RSSKSLLHSSGITMLS | SEQ ID |
| L1 | | NO: 16 |
| 61289 CDR- | QMSNLAS | SEQ ID |
| L2 | | NO: 17 |
| 61289 CDR- | AQSLELPLT | SEQ ID |
| L3 | | NO: 18 |
| 61289 VH | QVQLVQSGAEVKKPGASVKVSCKASGYTFTSSWIHWVRQ | SEQ ID |
| | APGQGLEWMGLIDPSDSYTNYNQKFRGRVTMTVDTSTST | NO: 19 |
| | AYMELSSLRSEDTAVYYCASSDPHGGWGQGTTVTVSS | |
| 61289 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 20 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61289 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 21 |
| | YEKHKVYACEVTHQGLSSPVTKSFNRGEC | |
| 61289 VL | DIVMTQSPLSLPVTPGEPASISCRSSKSLLHSSGITMLS | SEQ ID |
| | WYLQKPGQSPQLLIYQMSNLASGVPDRFSGSGSGTDFTL | NO: 22 |
| | KISRVEAEDVGVYYCAQSLELPLTFGGGTKVEIK | |
| 61289 | QVQLVQSGAEVKKPGASVKVSCKASGYTFTSSWIHWVRQ | SEQ ID |
| VH + CH | APGQGLEWMGLIDPSDSYTNYNQKFRGRVTMTVDTSTST | NO: 23 |
| | AYMELSSLRSEDTAVYYCASSDPHGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61289 | DIVMTQSPLSLPVTPGEPASISCRSSKSLLHSSGITMLS | SEQ ID |
| VL + CL | WYLQKPGQSPQLLIYQMSNLASGVPDRFSGSGSGTDFTL | NO: 24 |
| | KISRVEAEDVGVYYCAQSLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61275 | YTFTSSWIH | SEQ ID |
| CDR-H1 | | NO: 25 |
| 61275 | LIDPSDSYTNYNQKFRG | SEQ ID |
| CDR-H2 | | NO: 26 |
| 61275 | ASSAPTGG | SEQ ID |
| CDR-H3 | | NO: 27 |
| 61275 | RSSKSLLHSSGITYLS | SEQ ID |
| CDR-L1 | | NO: 28 |
| 61275 | QMSNLAS | SEQ ID |
| CDR-L2 | | NO: 29 |
| 61275 | AQALELPLT | SEQ ID |
| CDR-L3 | | NO: 30 |
| 61275 | QVQLVQSGAEVKKPGASVKVSCKASGYTFTSSWIHWVRQ | SEQ ID |
| VH | APGQGLEWMGLIDPSDSYTNYNQKFRGRVTMTVDTSTST | NO: 31 |
| | AYMELSSLRSEDTAVYYCASSAPTGGWGQGTTVTVSS | |
| 61275 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 32 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61275 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 33 |
| | YEKHKVYACEVTHQGLSSPVTKSFNRGEC | |
| 61275 | DIVMTQSPLSLPVTPGEPASISCRSSKSLLHSSGITYLS | SEQ ID |
| VL | WYLQKPGQSPQLLIYQMSNLASGVPDRFSGSGSGTDFTL | NO: 34 |
| | KISRVEAEDVGVYYCAQALELPLTFGGGTKVEIK | |
| 61275 | QVQLVQSGAEVKKPGASVKVSCKASGYTFTSSWIHWVRQ | SEQ ID |
| VH + CH | APGQGLEWMGLIDPSDSYTNYNQKFRGRVTMTVDTSTST | NO: 35 |
| | AYMELSSLRSEDTAVYYCASSAPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61275 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 36 |
| | KISRVEAEDVGVYYCAQALELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61278 | YTFTSYWIH | SEQ ID |
| CDR-H1 | | NO: 37 |
| 61278 CDR- | LIDPSDSYTNYNQKFRG | SEQ ID |
| H2 | | NO: 38 |
| 61278 CDR- | ASSDATGG | SEQ ID |
| H3 | | NO: 39 |
| 61278 CDR- | RSSKSLLHSSGITYLS | SEQ ID |
| L1 | | NO: 40 |
| 61278 CDR- | QMSNLAS | SEQ ID |
| L2 | | NO: 41 |
| 61278 CDR- | AQALELPLT | SEQ ID |
| L3 | | NO: 42 |
| 61278 VH | QVQLVQSGAEVKKPGASVKVSCKASGYTFTSYWIHWVRQ | SEQ ID |
| | APGQGLEWMGLIDPSDSYTNYNQKFRGRVTMTVDTSTST | NO: 43 |
| | AYMELSSLRSEDTAVYYCASSDATGGWGQGTTVTVSS | |
| 61278 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 44 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61278 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 45 |
| | YEKHKVYACEVTHQGLSSPVTKSFNRGEC | |
| 61278 VL | DIVMTQSPLSLPVTPGEPASISCRSSKSLLHSSGITYLS | SEQ ID |
| | WYLQKPGQSPQLLIYQMSNLASGVPDRFSGSGSGTDFTL | NO: 46 |
| | KISRVEAEDVGVYYCAQALELPLTFGGGTKVEIK | |
| 61278 | QVQLVQSGAEVKKPGASVKVSCKASGYTFTSYWIHWVRQ | SEQ ID |
| VH + CH | APGQGLEWMGLIDPSDSYTNYNQKFRGRVTMTVDTSTST | NO: 47 |
| | AYMELSSLRSEDTAVYYCASSDATGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61278 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 48 |
| | KISRVEAEDVGVYYCAQALELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61285 CDR- | YTFTSSWIH | SEQ ID |
| H1 | | NO: 49 |
| 61285 CDR- | LIDPSDSYTNYNQKFRG | SEQ ID |
| H2 | | NO: 50 |
| 61285 CDR- | ASSDPHGG | SEQ ID |
| H3 | | NO: 51 |
| 61285 CDR- | RSSKSLLHSSGITYLS | SEQ ID |
| L1 | | NO: 52 |
| 61285 CDR- | QMSNLAS | SEQ ID |
| L2 | | NO: 53 |
| 61285 CDR- | AQSLELPLT | SEQ ID |
| L3 | | NO: 54 |
| 61285 VH | QVQLVQSGAEVKKPGASVKVSCKASGYTFTSSWIHWVRQ | SEQ ID |
| | APGQGLEWMGLIDPSDSYTNYNQKFRGRVTMTVDTSTST | NO: 55 |
| | AYMELSSLRSEDTAVYYCASSDPHGGWGQGTTVTVSS | |
| 61285 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 56 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61285 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 57 |
| | YEKHKVYACEVTHQGLSSPVTKSFNRGEC | |
| 61285 VL | DIVMTQSPLSLPVTPGEPASISCRSSKSLLHSSGITYLS | SEQ ID |
| | WYLQKPGQSPQLLIYQMSNLASGVPDRFSGSGSGTDFTL | NO: 58 |
| | KISRVEAEDVGVYYCAQSLELPLTFGGGTKVEIK | |
| 61285 | QVQLVQSGAEVKKPGASVKVSCKASGYTFTSSWIHWVRQ | SEQ ID |
| VH + CH | APGQGLEWMGLIDPSDSYTNYNQKFRGRVTMTVDTSTST | NO: 59 |
| | AYMELSSLRSEDTAVYYCASSDPHGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61285 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 60 |
| | KISRVEAEDVGVYYCAQSLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61273 | YTFTSTWIH | SEQ ID |
| CDR-H1 | | NO: 61 |
| 61273 | LIDPSDSYTNYNQKFRG | SEQ ID |
| CDR-H2 | | NO: 62 |
| 61273 | ASSKPTGG | SEQ ID |
| CDR-H3 | | NO: 63 |
| 61273 | RSSKSLLHSSGITYIS | SEQ ID |
| CDR-L1 | | NO: 64 |
| 61273 | QMSNLAS | SEQ ID |
| CDR-L2 | | NO: 65 |
| 61273 | AQNLELPLT | SEQ ID |
| CDR-L3 | | NO: 66 |
| 61273 | QVQLVQSGAEVKKPGASVKVSCKASGYTFTSTWIHWVRQ | SEQ ID |
| VH | APGQGLEWMGLIDPSDSYTNYNQKFRGRVTMTVDTSTST | NO: 67 |
| | AYMELSSLRSEDTAVYYCASSKPTGGWGQGTTVTVSS | |
| 61273 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 68 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61273 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 69 |
| | YEKHKVYACEVTHQGLSSPVTKSFNRGEC | |
| 61273 | DIVMTQSPLSLPVTPGEPASISCRSSKSLLHSSGITYIS | SEQ ID |
| VL | WYLQKPGQSPQLLIYQMSNLASGVPDRFSGSGSGTDFTL | NO: 70 |
| | KISRVEAEDVGVYYCAQNLELPLTFGGGTKVEIK | |
| 61273 | QVQLVQSGAEVKKPGASVKVSCKASGYTFTSTWIHWVRQ | SEQ ID |
| VH + CH | APGQGLEWMGLIDPSDSYTNYNQKFRGRVTMTVDTSTST | NO: 71 |
| | AYMELSSLRSEDTAVYYCASSKPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61273 | DIVMTQSPLSLPVTPGEPASISCRSSKSLLHSSGITYIS | SEQ ID |
| VL + CL | WYLQKPGQSPQLLIYQMSNLASGVPDRFSGSGSGTDFTL | NO: 72 |
| | KISRVEAEDVGVYYCAQNLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 60183 | YTFTSTWIH | SEQ ID |
| CDR-H1 | | NO: 73 |
| 60183 | LIDPSDSYTNYNQKFRG | SEQ ID |
| CDR-H2 | | NO: 74 |
| 60183 | ASSKPTGG | SEQ ID |
| CDR-H3 | | NO: 75 |
| 60183 | RSSKSLLHSSGITYLS | SEQ ID |
| CDR-L1 | | NO: 76 |
| 60183 | QMSNLAS | SEQ ID |
| CDR-L2 | | NO: 77 |
| 60183 | AQNLELPLT | SEQ ID |
| CDR-L3 | | NO: 78 |
| 60183 | QVQLVQSGAEVKKPGASVKVSCKASGYTFTSTWIHWVRQ | SEQ ID |
| VH | APGQGLEWMGLIDPSDSYTNYNQKFRGRVTMTVDTSTST | NO: 79 |
| | AYMELSSLRSEDTAVYYCASSKPTGGWGQGTTVTVSS | |
| 60183 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 80 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 60183 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 81 |
| | YEKHKVYACEVTHQGLSSPVTKSFNRGEC | |
| 60183 | DIVMTQSPLSLPVTPGEPASISCRSSKSLLHSSGITYLS | SEQ ID |
| VL | WYLQKPGQSPQLLIYQMSNLASGVPDRFSGSGSGTDFTL | NO: 82 |
| | KISRVEAEDVGVYYCAQNLELPLTFGGGTKVEIK | |
| 60183 | QVQLVQSGAEVKKPGASVKVSCKASGYTFTSTWIHWVRQ | SEQ ID |
| VH + CH | APGQGLEWMGLIDPSDSYTNYNQKFRGRVTMTVDTSTST | NO: 83 |
| | AYMELSSLRSEDTAVYYCASSKPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 60183 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 84 |
| | KISRVEAEDVGVYYCAQNLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61283 | YTFTSAWIH | SEQ ID |
| CDR-H1 | | NO: 85 |
| 61283 | LIDPSDSYTNYNQKFRG | SEQ ID |
| CDR-H2 | | NO: 86 |
| 61283 | ASSDPYGG | SEQ ID |
| CDR-H3 | | NO: 87 |
| 61283 | RSSKSLLHSSGITYLS | SEQ ID |
| CDR-L1 | | NO: 88 |
| 61283 | QMSNKAS | SEQ ID |
| CDR-L2 | | NO: 89 |
| 61283 | AQSLELPLT | SEQ ID |
| CDR-L3 | | NO: 90 |
| 61283 | QVQLVQSGAEVKKPGASVKVSCKASGYTFTSAWIHWVRQ | SEQ ID |
| VH | APGQGLEWMGLIDPSDSYTNYNQKFRGRVTMTVDTSTST | NO: 91 |
| | AYMELSSLRSEDTAVYYCASSDPYGGWGQGTTVTVSS | |
| 61283 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 92 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61283 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 93 |
| | YEKHKVYACEVTHQGLSSPVTKSFNRGEC | |
| 61283 | DIVMTQSPLSLPVTPGEPASISCRSSKSLLHSSGITYLS | SEQ ID |
| VL | WYLQKPGQSPQLLIYQMSNKASGVPDRFSGSGSGTDFTL | NO: 94 |
| | KISRVEAEDVGVYYCAQSLELPLTFGGGTKVEIK | |
| 61283 | QVQLVQSGAEVKKPGASVKVSCKASGYTFTSAWIHWVRQ | SEQ ID |
| VH + CH | APGQGLEWMGLIDPSDSYTNYNQKFRGRVTMTVDTSTST | NO: 95 |
| | AYMELSSLRSEDTAVYYCASSDPYGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61283 | DIVMTQSPLSLPVTPGEPASISCRSSKSLLHSSGITYLS | SEQ ID |
| VL + CL | WYLQKPGQSPQLLIYQMSNKASGVPDRFSGSGSGTDFTL | NO: 96 |
| | KISRVEAEDVGVYYCAQSLELPLTFGGGTKVEIKQVQLV | |
| | QSGAEVKKPGASVKVSCKASGYTFTSAWIHWVRQAPGQG | |
| | LEWMGLIDPSDSYTNYNQKFRGRVTMTVDTSTSTAYMEL | |
| | SSLRSEDTAVYYCASSDPYGGWGQGTTVTVSSASTKGPS | |
| | VFPLAPSSKSTSGGTAALGCLVKDYFPEPVTVSWNSGAL | |
| | TSGVHTFPAVLQSSGLYSLSSVVTVPSSSLGTQTYICNV | |
| | NHKPSNTKVDKKVEPKSCDKTHTCPPCPAPELLGGPSVF | |
| | LFPPKPKDTLMISRTPEVTCVVVDVSHEDPEVKFNWYVD | |
| | GVEVHNAKTKPREEQYNSTYRVVSVLTVLHQDWLNGKEY | |
| | KCKVSNKALPAPIEKTISKAKGQPREPQVYTLPPSRDEL | |
| | TKNQVSLTCLVKGFYPSDIAVEWESNGQPENNYKTTPPV | |
| | LDSDGSFFLYSKLTVDKSRWQQGNVFSCSVMHEALHNHY | |
| | TQKSLSLSPG | |
| 61286 | YTFTSTWIH | SEQ ID |
| CDR-H1 | | NO: 97 |
| 61286 | LIDPSDSYTNYNQKFRG | SEQ ID |
| CDR-H2 | | NO: 98 |
| 61286 | ASSDLTGG | SEQ ID |
| CDR-H3 | | NO: 99 |
| 61286 | RSSKSLLHSSGITYLS | SEQ ID |
| CDR-L1 | | NO: 100 |
| 61286 | QMSNLAS | SEQ ID |
| CDR-L2 | | NO: 101 |
| 61286 | AQALELPLT | SEQ ID |
| CDR-L3 | | NO: 102 |
| 61286 | QVQLVQSGAEVKKPGASVKVSCKASGYTFTSTWIHWVRQ | SEQ ID |
| VH | APGQGLEWMGLIDPSDSYTNYNQKFRGRVTMTVDTSTST | NO: 103 |
| | AYMELSSLRSEDTAVYYCASSDLTGGWGQGTTVTVSS | |
| 61286 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 104 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61286 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 105 |
| | YEKHKVYACEVTHQGLSSPVTKSFNRGEC | |
| 61286 | DIVMTQSPLSLPVTPGEPASISCRSSKSLLHSSGITYLS | SEQ ID |
| VL | WYLQKPGQSPQLLIYQMSNLASGVPDRFSGSGSGTDFSL | NO: 106 |
| | KISRVEAEDVGVYYCAQALELPLTFGGGTKVEIK | |
| 61286 | QVQLVQSGAEVKKPGASVKVSCKASGYTFTSTWIHWVRQ | SEQ ID |
| VH + CH | APGQGLEWMGLIDPSDSYTNYNQKFRGRVTMTVDTSTST | NO: 107 |
| | AYMELSSLRSEDTAVYYCASSDLTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61286 | DIVMTQSPLSLPVTPGEPASISCRSSKSLLHSSGITYLS | SEQ ID |
| VL + CL | WYLQKPGQSPQLLIYQMSNLASGVPDRESGSGSGTDFSL | NO: 108 |
| | KISRVEAEDVGVYYCAQALELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61279 | YTFKSYWIH | SEQ ID |
| CDR-H1 | | NO: 109 |
| 61279 | LIDPSDSYTNYNQIFRG | SEQ ID |
| CDR-H2 | | NO: 110 |
| 61279 | ASSDATGG | SEQ ID |
| CDR-H3 | | NO: 111 |
| 61279 | RSSKSLLHSSGITYLS | SEQ ID |
| CDR-L1 | | NO: 112 |
| 61279 | QMSNLAS | SEQ ID |
| CDR-L2 | | NO: 113 |
| 61279 | AQALELPLT | SEQ ID |
| CDR-L3 | | NO: 114 |
| 61279 | QVQLVQSGAEVKKPGASVKVSCKASGYTFKSYWIHWVRQ | SEQ ID |
| VH | APGQGLEWMGLIDPSDSYTNYNQIFRGRVTMTVDTSTST | NO: 115 |
| | AYMELSSLRSEDTAVYYCASSDATGGWGQGTTVTVSS | |
| 61279 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 116 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61279 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 117 |
| | YEKHKVYACEVTHQGLSSPVTKSFNRGEC | |
| 61279 | DIVMTQSPLSLPVTPGEPASISCRSSKSLLHSSGITYLS | SEQ ID |
| VL | WYLQKPGQSPQLLIYQMSNLASGVPDRFSGSGSGTDFTL | NO: 118 |
| | KISRVEAEDVGVYYCAQALELPLTFGGGTKVEIK | |
| 61279 | QVQLVQSGAEVKKPGASVKVSCKASGYTFKSYWIHWVRQ | SEQ ID |
| VH + CH | APGQGLEWMGLIDPSDSYTNYNQIFRGRVTMTVDTSTST | NO: 119 |
| | AYMELSSLRSEDTAVYYCASSDATGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61279 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 120 |
| | KISRVEAEDVGVYYCAQALELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 56657 | YTFTSTWIH | SEQ ID |
| CDR-H1 | | NO: 121 |
| 56657 | LIDPSDSYTNYNQKFRG | SEQ ID |
| CDR-H2 | | NO: 122 |
| 56657 | ASSKPTGG | SEQ ID |
| CDR-H3 | | NO: 123 |
| 56657 | RSSKSLLHSSGITYLS | SEQ ID |
| CDR-L1 | | NO: 124 |
| 56657 | QMSNLAS | SEQ ID |
| CDR-L2 | | NO: 125 |
| 56657 | AQNLELPLT | SEQ ID |
| CDR-L3 | | NO: 126 |
| 56657 | QVQLVQSGAEVKKPGASVKVSCKASGYTFTSYWIHWVRQ | SEQ ID |
| VH | APGQGLEWIGLIDPSDSYTNYNQKFRGRATLTVDTSTST | NO: 127 |
| | AYMELSSLRSEDTAVYYCASSDPTGGWGQGTTVTVSS | |
| 56657 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 128 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 56657 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 129 |
| | YEKHKVYACEVTHQGLSSPVTKSFNRGEC | |
| 56657 | DIVMTQSPLSLPVTPGEPASISCRSSKSLLHSSGITYLS | SEQ ID |
| VL | WYLQKPGQSPQLLIYQMSNLASGVPDRESGSGSGTDFTL | NO: 130 |
| | KISRVEAEDVGVYYCAQNLELPLTFGGGTKVEIK | |
| 56657 | QVQLVQSGAEVKKPGASVKVSCKASGYTFTSYWIHWVRQ | SEQ ID |
| VH + CH | APGQGLEWIGLIDPSDSYTNYNQKFRGRATLTVDTSTST | NO: 131 |
| | AYMELSSLRSEDTAVYYCASSDPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 56657 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 132 |
| | KISRVEAEDVGVYYCAQNLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61255 CDR- | YTFTSYWIH | SEQ ID |
| H1 | | NO: 133 |
| 61255 | LIDPSDSYTNYNQKFRG | SEQ ID |
| CDR-H2 | | NO: 134 |
| 61255 | ASSDATGG | SEQ ID |
| CDR-H3 | | NO: 135 |
| 61255 | RSSKSLLHSSGHTYLS | SEQ ID |
| CDR-L1 | | NO: 136 |
| 61255 | QMSNLAS | SEQ ID |
| CDR-L2 | | NO: 137 |
| 61255 | AQALELPLT | SEQ ID |
| CDR-L3 | | NO: 138 |
| 61255 | QVQLVQSGAEVKKPGASVKVSCKASGYTFTSYWIHWVRQ | SEQ ID |
| VH | APGQGLEWIGLIDPSDSYTNYNQKFRGRATLTVDTSTST | NO: 139 |
| | AYMELSSLRSEDTAVYYCASSDATGGWGQGTTVTVSS | |
| 61255 | ASTKGPSVFPLAPSSKSTSGGTAALGCLVKDYFPEPVTV | SEQ ID |
| CH | SWNSGALTSGVHTFPAVLQSSGLYSLSSVVTVPSSSLGT | NO: 140 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61255 | RTVAAPSVFIFPPSDEQLKSGTASVVCLLNNFYPREAKV | SEQ ID |
| CL | QWKVDNALQSGNSQESVTEQDSKDSTYSLSSTLTLSKAD | NO: 141 |
| | YEKHKVYACEVTHQGLSSPVTKSFNRGEC | |
| 61255 | DIVMTQSPLSLPVTPGEPASISCRSSKSLLHSSGHTYLS | SEQ ID |
| VL | WYLQKPGQPPQLLIYQMSNLASGVPDRFSGSGSGTDFTL | NO: 142 |
| | KISRVEAEDVGVYYCAQALELPLTFGGGTKVEIK | |
| 61255 | QVQLVQSGAEVKKPGASVKVSCKASGYTFTSYWIHWVRQ | SEQ ID |
| VH + CH | APGQGLEWIGLIDPSDSYTNYNQKFRGRATLTVDTSTST | NO: 143 |
| | AYMELSSLRSEDTAVYYCASSDATGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61255 | DIVMTQSPLSLPVTPGEPASISCRSSKSLLHSSGHTYLS | SEQ ID |
| VL + CL | WYLQKPGQPPQLLIYQMSNLASGVPDRFSGSGSGTDFTL | NO: 144 |
| | KISRVEAEDVGVYYCAQALELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 60140 CDR- | YTFTSVWIH | SEQ ID |
| H1 | | NO: 145 |
| 60140 | LIDPSDSYTNYNQKFRG | SEQ ID |
| CDR-H2 | | NO: 146 |
| 60140 CDR- | ASSRPTGG | SEQ ID |
| H3 | | NO: 147 |
| 60140 CDR- | RSSKSLLHSSGITYLS | SEQ ID |
| L1 | | NO: 148 |
| 60140 CDR- | QMSNLAS | SEQ ID |
| L2 | | NO: 149 |
| 60140 CDR- | AQNLELPLT | SEQ ID |
| L3 | | NO: 150 |
| 60140 VH | QVQLVQSGAEVKKPGASVKVSCKASGYTFTSVWIHWVRQ | SEQ ID |
| | APGQGLEWIGLIDPSDSYTNYNQKFRGRATLTVDTSTST | NO: 151 |
| | AYMELSSLRSEDTAVYYCASSRPTGGWGQGTTVTVSS | |
| 60140 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 152 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 60140 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 153 |
| | YEKHKVYACEVTHQGLSSPVTKSFNRGEC | |
| 60140 VL | DIVMTQSPLSLPVTPGEPASISCRSSKSLLHSSGITYLS | SEQ ID |
| | WYLQKPGQSPQLLIYQMSNLASGVPDRFSGSGSGTDFTL | NO: 154 |
| | KISRVEAEDVGVYYCAQNLELPLTFGGGTKVEIK | |
| 60140 | QVQLVQSGAEVKKPGASVKVSCKASGYTFTSVWIHWVRQ | SEQ ID |
| VH + CH | APGQGLEWIGLIDPSDSYTNYNQKFRGRATLTVDTSTST | NO: 155 |
| | AYMELSSLRSEDTAVYYCASSRPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 60140 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 156 |
| | KISRVEAEDVGVYYCAQNLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 60143 CDR- | YTFTSTWIH | SEQ ID |
| H1 | | NO: 157 |
| 60143 | LIDPSDSYTNYNQKFRG | SEQ ID |
| CDR-H2 | | NO: 158 |
| 60143 CDR- | ASSKPTGG | SEQ ID |
| H3 | | NO: 159 |
| 60143 CDR- | RSSKSLLHSSGITYLS | SEQ ID |
| L1 | | NO: 160 |
| 60143 CDR- | QMSNLAS | SEQ ID |
| L2 | | NO: 161 |
| 60143 CDR- | AQNLELPLT | SEQ ID |
| L3 | | NO: 162 |
| 60143 VH | QVQLVQSGAEVKKPGASVKVSCKASGYTFTSTWIHWVRQ | SEQ ID |
| | APGQGLEWIGLIDPSDSYTNYNQKFRGRATLTVDTSTST | NO: 163 |
| | AYMELSSLRSEDTAVYYCASSKPTGGWGQGTTVTVSS | |
| 60143 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 164 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 60143 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 165 |
| | YEKHKVYACEVTHQGLSSPVTKSFNRGEC | |
| 60143 VL | DIVMTQSPLSLPVTPGEPASISCRSSKSLLHSSGITYLS | SEQ ID |
| | WYLQKPGQSPQLLIYQMSNLASGVPDRFSGSGSGTDFTL | NO: 166 |
| | KISRVEAEDVGVYYCAQNLELPLTFGGGTKVEIK | |
| 60143 | QVQLVQSGAEVKKPGASVKVSCKASGYTFTSTWIHWVRQ | SEQ ID |
| VH + CH | APGQGLEWIGLIDPSDSYTNYNQKFRGRATLTVDTSTST | NO: 167 |
| | AYMELSSLRSEDTAVYYCASSKPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 60143 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 168 |
| | KISRVEAEDVGVYYCAQNLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61264 CDR- | YTFTSTWIH | SEQ ID |
| H1 | | NO: 169 |
| 61264 | LIDPSDSYTNYNQKFVG | SEQ ID |
| CDR-H2 | | NO: 170 |
| 61264 CDR- | ASSLPTGG | SEQ ID |
| H3 | | NO: 171 |
| 61264 CDR- | RSSKSLLHSSGITYLS | SEQ ID |
| L1 | | NO: 172 |
| 61264 CDR- | QMSNLAS | SEQ ID |
| L2 | | NO: 173 |
| 61264 CDR- | AQQLELPLT | SEQ ID |
| L3 | | NO: 174 |
| 61264 VH | QVQLVQSGAEVKKPGASVKVSCKASGYTFTSTWIHWVRQ | SEQ ID |
| | APGQGLEWIGLIDPSDSYTNYNQKFVGRATLTVDTSTST | NO: 175 |
| | AYMELSSLRSEDTAVYYCASSLPTGGWGQGTTVTVSS | |
| 61264 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 176 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61264 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 177 |
| | YEKHKVYACEVTHQGLSSPVTKSFNRGEC | |
| 61264 VL | DIVMTQSPLSLPVTPGEPASISCRSSKSLLHSSGITYLS | SEQ ID |
| | WYLQKPGQSPQLLIYQMSNLASGVPDRFSGSGSGTDFTL | NO: 178 |
| | KISRVEAEDVGVYYCAQQLELPLTFGGGTKVEIK | |
| 61264 | QVQLVQSGAEVKKPGASVKVSCKASGYTFTSTWIHWVRQ | SEQ ID |
| VH + CH | APGQGLEWIGLIDPSDSYTNYNQKFVGRATLTVDTSTST | NO: 179 |
| | AYMELSSLRSEDTAVYYCASSLPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61264 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 180 |
| | KISRVEAEDVGVYYCAQQLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 60130 CDR- | YTFTSVWIH | SEQ ID |
| H1 | | NO: 181 |
| 60130 | LIDPSDSYTNYNQKFR | SEQ ID |
| CDR-H2 | | NO: 182 |
| 60130 | ASSQPTGG | SEQ ID |
| CDR-H3 | | NO: 183 |
| 60130 | RSSKSLLHSSGITYLS | SEQ ID |
| CDR-L1 | | NO: 184 |
| 60130 | QMSNLAS | SEQ ID |
| CDR-L2 | | NO: 185 |
| 60130 CDR- | AQNLELPLT | SEQ ID |
| L3 | | NO: 186 |
| 60130 VH | QVQLVQSGAEVKKPGASVKVSCKASGYTFTSVWIHWVRQ | SEQ ID |
| | APGQGLEWIGLIDPSDSYTNYNQKFRGRATLTVDTSTST | NO: 187 |
| | AYMELSSLRSEDTAVYYCASSQPTGGWGQGTTVTVSS | |
| 60130 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 188 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 60130 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 189 |
| | YEKHKVYACEVTHQGLSSPVTKSFNRGEC | |
| 60130 VL | DIVMTQSPLSLPVTPGEPASISCRSSKSLLHSSGITYLS | SEQ ID |
| | WYLQKPGQSPQLLIYQMSNLASGVPDRFSGSGSGTDFTL | NO: 190 |
| | KISRVEAEDVGVYYCAQNLELPLTFGGGTKVEIK | |
| 60130 | QVQLVQSGAEVKKPGASVKVSCKASGYTFTSVWIHWVRQ | SEQ ID |
| VH + CH | APGQGLEWIGLIDPSDSYTNYNQKFRGRATLTVDTSTST | NO: 191 |
| | AYMELSSLRSEDTAVYYCASSQPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 60130 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 192 |
| | KISRVEAEDVGVYYCAQNLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQ | |
| | GLSSPVTKSFNRGEC | |
| 61286 CDR- | YTFTSTWIH | SEQ ID |
| H1 | | NO: 193 |
| 61286 | LIDPSDSYTNYNQKFR | SEQ ID |
| CDR-H2 | | NO: 194 |
| 61286 | ASSDLTGG | SEQ ID |
| CDR-H3 | | NO: 195 |
| 61286 | RSSKSLLHSSGITYLS | SEQ ID |
| CDR-L1 | | NO: 196 |
| 61286 | QMSNLAS | SEQ ID |
| CDR-L2 | | NO: 197 |
| 61286 | AQALELPLT | SEQ ID |
| CDR-L3 | | NO: 198 |
| 61286 VH | QVQLVQSGAEVKKPGASVKVSCKASGYTFTSTWIHWVRQ | SEQ ID |
| | APGQGLEWMGLIDPSDSYTNYNQKFRGRVTMTVDTSTST | NO: 199 |
| | AYMELSSLRSEDTAVYYCASSDLTGGWGQGTTVTVSS | |
| 61286 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 200 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61286 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 201 |
| | YEKHKVYACEVTHQGLSSPVTKSFNRGEC | |
| 61286 VL | DIVMTQSPLSLPVTPGEPASISCRSSKSLLHSSGITYLS | SEQ ID |
| | WYLQKPGQSPQLLIYQMSNLASGVPDRESGSGSGTDFSL | NO: 202 |
| | KISRVEAEDVGVYYCAQALELPLTFGGGTKVEIK | |
| 61286 | QVQLVQSGAEVKKPGASVKVSCKASGYTFTSTWIHWVRQ | SEQ ID |
| VH + CH | APGQGLEWMGLIDPSDSYTNYNQKFRGRVTMTVDTSTST | NO: 203 |
| | AYMELSSLRSEDTAVYYCASSDLTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61286 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFSL | NO: 204 |
| | KISRVEAEDVGVYYCAQALELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61259 CDR- | YRFTSYWIH | SEQ ID |
| H1 | | NO: 205 |
| 61259 | LIDPSDSYTNYNQKFRG | SEQ ID |
| CDR-H2 | | NO: 206 |
| 61259 | ASSDATGG | SEQ ID |
| CDR-H3 | | NO: 207 |
| 61259 | VSSKSLLHSSGITYLS | SEQ ID |
| CDR-L1 | | NO: 208 |
| 61259 | QMSNLGS | SEQ ID |
| CDR-L2 | | NO: 209 |
| 61259 | AQALELPLT | SEQ ID |
| CDR-L3 | | NO: 210 |
| 61259 VH | QVQLVQSGAEVKKPGASVKVSCKASGYRFTSYWIHWVRQ | SEQ ID |
| | APGQGLEWIGLIDPSDSYTNYNQKFRGRATLTVDTSTST | NO: 211 |
| | AYMELSSLRSEDTAVYYCASSDATGGWGQGTTVTVSS | |
| 61259 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 212 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61259 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 213 |
| | YEKHKVYACEVTHQGLSSPVTKSFNRGEC | |
| 61259 VL | DIVMTQSPLSLPVTPGEPASISCVSSKSLLHSSGITYLS | SEQ ID |
| | WYLQKPGQSPQLLIYQMSNLGSGVPDRFSGSGSGTDFTL | NO: 214 |
| | KISRVEAEDVGVYYCAQALELPLTFGGGTKVEIK | |
| 61259 | QVQLVQSGAEVKKPGASVKVSCKASGYRFTSYWIHWVRQ | SEQ ID |
| VH + CH | APGQGLEWIGLIDPSDSYTNYNQKFRGRATLTVDTSTST | NO: 215 |
| | AYMELSSLRSEDTAVYYCASSDATGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61259 | DIVMTQSPLSLPVTPGEPASISCVSSKSLLHSSGITYLS | SEQ ID |
| VL + CL | WYLQKPGQSPQLLIYQMSNLGSGVPDRFSGSGSGTDFTL | NO: 216 |
| | KISRVEAEDVGVYYCAQALELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 60136 CDR- | YTHTSYWIH | SEQ ID |
| H1 | | NO: 217 |
| 60136 | LIDPSDSYTNYNQKFR | SEQ ID |
| CDR-H2 | | NO: 218 |
| 60136 | ASSRPTGG | SEQ ID |
| CDR-H3 | | NO: 219 |
| 60136 | RSSKSLLHSSGITYLS | SEQ ID |
| CDR-L1 | | NO: 220 |
| 60136 | QMSNLAS | SEQ ID |
| CDR-L2 | | NO: 221 |
| 60136 | AQNLELPLT | SEQ ID |
| CDR-L3 | | NO: 222 |
| 60136 VH | QVQLVQSGAEVKKPGASVKVSCKASGYTHTSYWIHWVRQ | SEQ ID |
| | APGQGLEWIGLIDPSDSYTNYNQKFRGRATLTVDTSTST | NO: 223 |
| | AYMELSSLRSEDTAVYYCASSRPTGGWGQGTTVTVSS | |
| 60136 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 224 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 60136 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 225 |
| | YEKHKVYACEVTHQGLSSPVTKSFNRGEC | |
| 60136 VL | DIVMTQSPLSLPVTPGEPASISCRSSKSLLHSSGITYLS | SEQ ID |
| | WYLQKPGQSPQLLIYQMSNLASGVPDRFSGSGSGTDFTL | NO: 226 |
| | KISRVEAEDVGVYYCAQNLELPLTFGGGTKVEIK | |
| 60136 | QVQLVQSGAEVKKPGASVKVSCKASGYTHTSYWIHWVRQ | SEQ ID |
| VH + CH | APGQGLEWIGLIDPSDSYTNYNQKFRGRATLTVDTSTST | NO: 227 |
| | AYMELSSLRSEDTAVYYCASSRPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 60136 | DIVMTQSPLSLPVTPGEPASISCRSSKSLLHSSGITYLS | SEQ ID |
| VL + CL | WYLQKPGQSPQLLIYQMSNLASGVPDRFSGSGSGTDFTL | NO: 228 |
| | KISRVEAEDVGVYYCAQNLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| 61267 CDR- | YTFTEYWIH | SEQ ID |
| H1 | | NO: 229 |
| 61267 | LIDPSDSYTNYNQRFR | SEQ ID |
| CDR-H2 | | NO: 230 |
| 61267 | ASSDATGG | SEQ ID |
| CDR-H3 | | NO: 231 |
| 61267 | HSSKSLLHSSGITYLS | SEQ ID |
| CDR-L1 | | NO: 232 |
| 61267 | QMSNLAS | SEQ ID |
| CDR-L2 | | NO: 233 |
| 61267 | AQSLELPLT | SEQ ID |
| CDR-L3 | | NO: 234 |
| 61267 VH | QVQLVQSGAEVKKPGASVKASCKASGYTFTEYWIHWVRQ | SEQ ID |
| | APGQGLEWIGLIDPSDSYTNYNQRFRGRATLTVDTSTST | NO: 235 |
| | AYMELSSLRSEDTAVYYCASSDATGGWGQGTTVTVSS | |
| 61267 CH | ASTKGPSVFPLAPSSKSTSGGTAALGCLVKDYFPEPVTV | SEQ ID |
| | SWNSGALTSGVHTFPAVLQSSGLYSLSSVVTVPSSSLGT | NO: 236 |
| | QTYICNVNHKPSNTKVDKKVEPKSCDKTHTCPPCPAPEL | |
| | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 61267 CL | RTVAAPSVFIFPPSDEQLKSGTASVVCLLNNFYPREAKV | SEQ ID |
| | QWKVDNALQSGNSQESVTEQDSKDSTYSLSSTLTLSKAD | NO: 237 |
| | YEKHKVYACEVTHQGLSSPVTKSENRGEC | |
| 61267 VL | DIVMTQSPLSLPVTPGEPASISCHSSKSLLHSSGITYLS | SEQ ID |
| | WYLQKPGQSPQLLIYQMSNLASGVPDRFSGSGSGTDFTL | NO: 238 |
| | KISRVEAEDVGVYYCAQSLELPLTFGGGTKVEIK | |
| 61267 | QVQLVQSGAEVKKPGASVKASCKASGYTFTEYWIHWVRQ | SEQ ID |
| VH + CH | APGQGLEWIGLIDPSDSYTNYNQRFRGRATLTVDTSTST | NO: 239 |
| | AYMELSSLRSEDTAVYYCASSDATGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKKVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 61267 | DIVMTQSPLSLPVTPGEPASISCHSSKSLLHSSGITYLS | SEQ ID |
| VL + CL | WYLQKPGQSPQLLIYQMSNLASGVPDRFSGSGSGTDFTL | NO: 240 |
| | KISRVEAEDVGVYYCAQSLELPLTFGGGTKVEIKRTVAA | |
| | PSVFIFPPSDEQLKSGTASVVCLLNNFYPREAKVQWKVD | |
| | NALQSGNSQESVTEQDSKDSTYSLSSTLTLSKADYEKHK | |
| | VYACEVTHQGLSSPVTKSFNRGEC | |
| Human | YSMKKTTMKIIPFNRLTIG | SEQ ID |
| fibrin γ377- | | NO: 241 |
| 395 | | |
| (Human P2 | | |
| peptide) | | |
| 60143 Light | ATGACTAGGCTGACCGTGCTGGCCCTGCTTGCCGGACTC | SEQ ID |
| Chain | TTGGCCTCCTCGAGAGCGGATATTGTGATGACTCAGAGC | NO: 242 |
| | CCACTCTCCCTGCCCGTGACTCCTGGGGAACCCGCCTCG | |
| | ATCAGCTGTAGATCGTCCAAGTCACTTCTCCACTCGTCC | |
| | GGGATCACCTACCTGTCGTGGTATTTGCAAAAGCCAGGA | |
| | CAGAGCCCGCAGCTCCTCATCTACCAAATGAGCAACCTG | |
| | GCTTCCGGTGTCCCGGATCGGTTCTCGGGGTCCGGATCT | |
| | GGCACCGACTTCACGCTGAAAATTTCCCGCGTGGAAGCC | |
| | GAGGACGTGGGAGTGTACTACTGCGCACAAAACCTGGAA | |
| | CTGCCCCTGACCTTCGGTGGCGGCACTAAGGTCGAAATC | |
| | AAGCGGACCGTGGCAGCTCCGTCCGTGTTCATCTTCCCG | |
| | CCTTCCGACGAGCAGCTGAAGTCCGGAACCGCCTCCGTC | |
| | GTGTGCCTGCTCAACAACTTTTACCCTCGCGAGGCCAAG | |
| | GTCCAGTGGAAGGTCGATAACGCGCTGCAGAGCGGAAAT | |
| | AGCCAGGAGAGCGTGACCGAGCAGGACTCCAAGGACTCA | |
| | ACCTACTCACTGAGCTCCACTCTGACCCTGTCAAAGGCG | |
| | GACTACGAGAAGCACAAAGTGTACGCCTGCGAAGTGACA | |
| | CATCAGGGCCTGTCCAGTCCCGTGACCAAGTCCTTCAAC | |
| | CGGGGCGAATGCTAG | |
| 60143 | ATGACCCGGCTGACCGTGCTGGCCCTCCTGGCTGGACTG | SEQ ID |
| Heavy Chain | CTGGCCTCCTCAAGAGCCCAGGTCCAGCTGGTGCAATCC | NO: 243 |
| | GGCGCCGAAGTCAAGAAGCCAGGCGCAAGCGTGAAAGTG | |
| | TCATGCAAAGCCTCCGGATACACCTTCACCTCCACCTGG | |
| | ATTCACTGGGTCAGACAGGCCCCCGGTCAAGGACTGGAA | |
| | TGGATCGGGCTGATCGACCCGTCGGACTCGTACACCAAC | |
| | TACAATCAGAAGTTTCGCGGTCGGGCTACTCTCACTGTG | |
| | GATACCTCGACCTCCACCGCTTACATGGAACTGTCATCG | |
| | CTGCGGTCCGAGGATACCGCCGTGTACTATTGCGCGTCC | |
| | TCCAAGCCGACTGGCGGATGGGGACAGGGAACTACTGTG | |
| | ACGGTGTCCTCCGCCTCGACCAAGGGCCCCTCCGTGTTT | |
| | CCACTGGCCCCCTCATCCAAGTCTACCAGCGGAGGAACC | |
| | GCAGCCCTAGGCTGTCTCGTGAAGGACTACTTCCCCGAG | |
| | CCGGTCACTGTCTCCTGGAACTCGGGAGCCCTCACTAGC | |
| | GGTGTCCACACTTTCCCGGCGGTGTTGCAAAGCTCCGGG | |
| | CTGTACTCCCTGTCCTCGGTCGTCACCGTGCCGTCAAGC | |
| | TCCCTCGGGACCCAGACATACATCTGTAACGTCAACCAT | |
| | AAGCCATCCAACACCAAAGTGGACAAGAAAGTGGAGCCG | |
| | AAAAGCTGCGACAAGACTCACACTTGCCCTCCTTGCCCT | |
| | GCACCCGAGCTTCTCGGAGGTCCCAGCGTGTTCCTGTTC | |
| | CCGCCGAAGCCCAAGGACACTCTGATGATTAGCCGCACT | |
| | CCTGAGGTCACCTGTGTCGTGGTGGACGTGTCCCATGAG | |
| | GACCCTGAAGTCAAGTTCAATTGGTACGTGGACGGCGTG | |
| | GAGGTGCACAACGCCAAGACCAAGCCGAGGGAGGAGCAG | |
| | TACAACTCGACCTATCGCGTGGTGTCCGTGCTCACCGTG | |
| | CTGCATCAGGATTGGCTGAACGGGAAGGAGTATAAGTGC | |
| | AAAGTGTCCAACAAGGCTTTGCCGGCCCCTATCGAAAAG | |
| | ACCATTAGCAAGGCCAAGGGGCAGCCAAGGGAGCCTCAA | |
| | GTGTACACCCTGCCGCCTTCGAGAGATGAACTGACCAAG | |
| | AACCAAGTGTCCCTCACGTGCCTCGTGAAGGGCTTCTAC | |
| | CCCTCCGATATCGCGGTGGAATGGGAATCCAACGGACAG | |
| | CCCGAAAACAACTACAAGACCACCCCTCCGGTGCTTGAT | |
| | AGCGACGGCTCGTTCTTCCTGTACTCGAAGCTGACAGTG | |
| | GACAAGTCACGGTGGCAGCAGGGCAACGTGTTCTCATGC | |
| | TCCGTGATGCACGAAGCGTTGCACAATCACTACACCCAG | |
| | AAGTCGCTTAGCCTGAGCCCTGGATAG | |
| 60143- | ATGACTAGGCTGACCGTGCTGGCCCTGCTTGCCGGACTC | SEQ ID |
| A234LA235 | TTGGCCTCCTCGAGAGCGGATATTGTGATGACTCAGAGC | NO: 244 |
| L Light | CCACTCTCCCTGCCCGTGACTCCTGGGGAACCCGCCTCG | |
| Chain | ATCAGCTGTAGATCGTCCAAGTCACTTCTCCACTCGTCC | |
| | GGGATCACCTACCTGTCGTGGTATTTGCAAAAGCCAGGA | |
| | CAGAGCCCGCAGCTCCTCATCTACCAAATGAGCAACCTG | |
| | GCTTCCGGTGTCCCGGATCGGTTCTCGGGGTCCGGATCT | |
| | GGCACCGACTTCACGCTGAAAATTTCCCGCGTGGAAGCC | |
| | GAGGACGTGGGAGTGTACTACTGCGCACAAAACCTGGAA | |
| | CTGCCCCTGACCTTCGGTGGCGGCACTAAGGTCGAAATC | |
| | AAGCGGACCGTGGCAGCTCCGTCCGTGTTCATCTTCCCG | |
| | CCTTCCGACGAGCAGCTGAAGTCCGGAACCGCCTCCGTC | |
| | GTGTGCCTGCTCAACAACTTTTACCCTCGCGAGGCCAAG | |
| | GTCCAGTGGAAGGTCGATAACGCGCTGCAGAGCGGAAAT | |
| | AGCCAGGAGAGCGTGACCGAGCAGGACTCCAAGGACTCA | |
| | ACCTACTCACTGAGCTCCACTCTGACCCTGTCAAAGGCG | |
| | GACTACGAGAAGCACAAAGTGTACGCCTGCGAAGTGACA | |
| | CATCAGGGCCTGTCCAGTCCCGTGACCAAGTCCTTCAAC | |
| | CGGGGCGAATGCTAG | |
| 60143- | ATGACCCGGCTGACCGTGCTGGCCCTCCTGGCTGGACTG | SEQ ID |
| A234LA235 | CTGGCCTCCTCAAGAGCCCAGGTCCAGCTGGTGCAATCC | NO: 245 |
| L Heavy | GGCGCCGAAGTCAAGAAGCCAGGCGCAAGCGTGAAAGTG | |
| Chain | TCATGCAAAGCCTCCGGATACACCTTCACCTCCACCTGG | |
| | ATTCACTGGGTCAGACAGGCCCCCGGTCAAGGACTGGAA | |
| | TGGATCGGGCTGATCGACCCGTCGGACTCGTACACCAAC | |
| | TACAATCAGAAGTTTCGCGGTCGGGCTACTCTCACTGTG | |
| | GATACCTCGACCTCCACCGCTTACATGGAACTGTCATCG | |
| | CTGCGGTCCGAGGATACCGCCGTGTACTATTGCGCGTCC | |
| | TCCAAGCCGACTGGCGGATGGGGACAGGGAACTACTGTG | |
| | ACGGTGTCCTCCGCCTCGACCAAGGGCCCCTCCGTGTTT | |
| | CCACTGGCCCCCTCATCCAAGTCTACCAGCGGAGGAACC | |
| | GCAGCCCTAGGCTGTCTCGTGAAGGACTACTTCCCCGAG | |
| | CCGGTCACTGTCTCCTGGAACTCGGGAGCCCTCACTAGC | |
| | GGTGTCCACACTTTCCCGGCGGTGTTGCAAAGCTCCGGG | |
| | CTGTACTCCCTGTCCTCGGTCGTCACCGTGCCGTCAAGC | |
| | TCCCTCGGGACCCAGACATACATCTGTAACGTCAACCAT | |
| | AAGCCATCCAACACCAAAGTGGACAAGAAAGTGGAGCCG | |
| | AAAAGCTGCGACAAGACTCACACTTGCCCTCCTTGCCCT | |
| | GCACCCGAGGCAGCAGGAGGTCCCAGCGTGTTCCTGTTC | |
| | CCGCCGAAGCCCAAGGACACTCTGATGATTAGCCGCACT | |
| | CCTGAGGTCACCTGTGTCGTGGTGGACGTGTCCCATGAG | |
| | GACCCTGAAGTCAAGTTCAATTGGTACGTGGACGGCGTG | |
| | GAGGTGCACAACGCCAAGACCAAGCCGAGGGAGGAGCAG | |
| | TACAACTCGACCTATCGCGTGGTGTCCGTGCTCACCGTG | |
| | CTGCATCAGGATTGGCTGAACGGGAAGGAGTATAAGTGC | |
| | AAAGTGTCCAACAAGGCTTTGCCGGCCCCTATCGAAAAG | |
| | ACCATTAGCAAGGCCAAGGGGCAGCCAAGGGAGCCTCAA | |
| | GTGTACACCCTGCCGCCTTCGAGAGATGAACTGACCAAG | |
| | AACCAAGTGTCCCTCACGTGCCTCGTGAAGGGCTTCTAC | |
| | CCCTCCGATATCGCGGTGGAATGGGAATCCAACGGACAG | |
| | CCCGAAAACAACTACAAGACCACCCCTCCGGTGCTTGAT | |
| | AGCGACGGCTCGTTCTTCCTGTACTCGAAGCTGACAGTG | |
| | GACAAGTCACGGTGGCAGCAGGGCAACGTGTTCTCATGC | |
| | TCCGTGATGCACGAAGCGTTGCACAATCACTACACCCAG | |
| | AAGTCGCTTAGCCTGAGCCCTGGATAG | |
| 5B8 | QVQLQQPGAELVRPGTSVKLSCKASGYTFTSYWIHWVKQ | SEQ ID |
| Heavy Chain | RPGQGLEWIGLIDPSDSYTNYNQKFRGKATLTVDTSSST | NO: 246 |
| | AYMQLSSLTSEDSAVYYCASSDPTGCWGQGTTLTVSSAK | |
| | TTPPSVYPLAPGCGDTTGSSVTSGCLVKGYFPEPVTVTW | |
| | NSGSLSSSVHTFPALLQSGLYTMSSSVTVPSSTWPSQTV | |
| | TCSVAHPASSTTVDKKLEPSGPISTINPCPPCKECHKCP | |
| | APNLEGGPSVFIFPPNIKDVLMISLTPKVTCVVVDVSED | |
| | DPDVQISWFVNNVEVHTAQTQTHREDYNSTIRVVSTLPI | |
| | QHQDWMSGKEFKCKVNNKDLPSPIERTISKIKGLVRAPQ | |
| | VYTLPPPAEQLSRKDVSLTCLVVGFNPGDISVEWTSNGH | |
| | TEENYKDTAPVLDSDGSYFIYSKLNMKTSKWEKTDSFSC | |
| | NVRHEGLKNYYLKKTISRSPGK | |
| 5B8 | RADAAPTVSIFPPSSEQLTSGGASVVCFLNNFYPKDINV | SEQ ID |
| Light Chain | KWKIDGSERQNGVLNSWTDQDSKDSTYSMSSTLTLTKDE | NO: 246 |
| | YERHNSYTCEATHKTSTSPIVKSFNRNECDIVMTQAAFS | |
| | NPITLGTSASMSCRSSKSLLHSSGITYLSWYLQKPGQSP | |
| | QLLIYQMSNLASGVPDRESSSGSGTDFTLRISRVEAEDV | |
| | GVYYCAQNLELPLTFGAGTKLELK | |
| THN227 | QVQLVQSGAEVKKPGSSVKVSCKASGYTFTSYWIHWVRQ | SEQ ID |
| Heavy Chain | APGQGLEWIGLIDPSDSYTNYNQKFRGRVTITRDTSTST | NO: 247 |
| | AYMELSSLRSEDTAVYYCASSDPTGGWGQGTTVTVSSAS | |
| | TKGPSVFPLAPSSKSTSGGTAALGCLVKDYFPEPVTVSW | |
| | NSGALTSGVHTFPAVLQSSGLYSLSSVVTVPSSSLGTQT | |
| | YICNVNHKPSNTKVDKRVEPKSCDKTHTCPPCPAPELLG | |
| | GPSVFLFPPKPKDTLMISRTPEVTCVVVDVSHEDPEVKF | |
| | NWYVDGVEVHNAKTKPREEQYNSTYRVVSVLTVLHQDWL | |
| | NGKEYKCKVSNKALPAPIEKTISKAKGQPREPQVYTLPP | |
| | SREEMTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPGK | |
| THN227 | RTVAAPSVFIFPPSDEQLKSGTASVVCLLNNFYPREAKV | SEQ ID |
| Light Chain | QWKVDNALQSGNSQESVTEQDSKDSTYSLSSTLTLSKAD | NO: 248 |
| | YEKHKVYACEVTHQGLSSPVTKSFNRGECDIVMTQAAFS | |
| | NPVTPGTPASISCRSSKSLLHSSGITYLSWYLQKPGQSP | |
| | QLLIYQMSNLASGVPDRESSSGSGTDFTLKISRVEAEDV | |
| | GVYYCAQNLELPLTFGQGTKLEIK | |
| Mouse/Rat | YSMKETTMKIIPFNRLSIG | SEQ ID |
| P2 peptide | | NO: 249 |
| Rabbit P2 | YSMKKTTMKIIPLNRLSIG | SEQ ID |
| peptide | | NO: 250 |
| Human P2 | TTMKIIPFNRLTIGEGQQHHLGGAKQVRP | SEQ ID |
| peptide | | NO: 251 |
| extended | | |
| Rat P2 | TTMKIIPFNRLSIGDGQQHHMGGSKQVSV | SEQ ID |
| peptide | | NO: 252 |
| extended | | |
| Rabbit P2 | TTMKIIPLNRLSIGEGQQFHVGGAKQVRP | SEQ ID |
| peptide | | NO: 253 |
| extended | | |
| THN420 | QVQLQQSGPELVRPGTSVRMSCKASGYTFTRYWMHWVKQ | SEQ ID |
| Heavy Chain | RPGQGLEWIGMIDPSNSEIRLNQKFLDKATLNVDKSSNT | NO: 254 |
| | AYMHLSSLTSEDSGVYYCARADWDRDSWGQGTTLTVSSA | |
| | KTTPPSVYPLAPGSAAQTNSMVTLGCLVKGYFPEPVTVT | |
| | WNSGSLSSGVHTFPAVLQSDLYTLSSSVTVPSSTWPSET | |
| | VTCNVAHPASSTKVDKKIVPRDCGCKPCICTVPEVSSVF | |
| | IFPPKPKDVLTITLTPKVTCVVVDISKDDPEVQFSWFVD | |
| | DVEVHTAQTQPREEQFNSTFRSVSELPIMHQDWLNGKEF | |
| | KCRVNSAAFPAPIEKTISKTKGRPKAPQVYTIPPPKEQM | |
| | AKDKVSLTCMITDFFPEDITVEWQWNGQPAENYKNTQPI | |
| | MDTDGSYFVYSKLNVQKSNWEAGNTFTCSVLHEGLHNHH | |
| | TEKSLSHSPG | |
| THN420 | RADAAPTVSIFPPSSEQLTSGGASVVCFLNNFYPKDINV | SEQ ID |
| Light Chain | KWKIDGSERQDGVLNSWTDQDSKDSTYSMSSTLTLTKDE | NO: 255 |
| | YERHNSYTCEATHKTSTSPIVKSFNRNECDIVMSQSPSS | |
| | LAVSAGEKVTMTCKSSQSLLNSRTRKNYLAWYQEKPGQS | |
| | PKLLIYWASTRESGVPDRFTGSGSGTDFTLTISSVQAED | |
| | LAVYYCKQSYTLRTFGGGTKLEIK | |
| THN420 | QVQLQQSGPELVRPGTSVRMSCKASGYTFTRYWMHWVKQ | SEQ ID |
| VH | RPGQGLEWIGMIDPSNSEIRLNQKFLDKATLNVDKSSNT | NO: 256 |
| | AYMHLSSLTSEDSGVYYCARADWDRDSWGQGTTLTVSS | |
| THN420 | DIVMSQSPSSLAVSAGEKVTMTCKSSQSLLNSRTRKNYL | SEQ ID |
| VL | AWYQEKPGQSPKLLIYWASTRESGVPDRFTGSGSGTDFT | NO: 257 |
| | LTISSVQAEDLAVYYCKQSYTLRTFGGGTKLEIK | |
| THN420 | GYTFTRYWMH | SEQ ID |
| CDR-H1 | | NO: 258 |
| THN420 | MIDPSNSEIRLNQKFLD | SEQ ID |
| CDR-H2 | | NO: 259 |
| THN420 | ADWDRDS | SEQ ID |
| CDR-H3 | | NO: 260 |
| THN420 | KSSQSLLNSRTRKNYLA | SEQ ID |
| CDR-L1 | | NO: 262 |
| THN420 | WASTRES | SEQ ID |
| CDR-L2 | | NO: 263 |
| THN420 | KQSYTLRT | SEQ ID |
| CDR-L3 | | NO: 264 |
| VEGF-A | MNFLLSWVHWSLALLLYLHHAKWSQAAPMAEGGGQNHHE | SEQ ID |
| | VVKFMDVYQRSYCHPIETLVDIFQEYPDEIEYIFKPSC | NO: 265 |
| | VPLMRCGGCCNDEGLECVPTEESNITMQIMRIKPHQGQH | |
| | IGEMSFLQHNKCECRPKKDRARQENPCGPCSERRKHLFV | |
| | QDPQTCKCSCKNTDSRCKARQLELNERTCRCDKPRR | |
| Aflibercept | SDTGRPFVEMYSEIPEIIHMTEGRELVIPCRVTSPNIT | SEQ ID |
| | VTLKKFPLDTLIPDGKRIIWDSRKGFIISNATYKEIGLL | NO: 266 |
| | TCEATVNGHLYKTNYLTHRQTNTIIDVVLSPSHGIELSV | |
| | GEKLVLNCTARTELNVGIDFNWEYPSSKHQHKKLVNRDL | |
| | KTQSGSEMKKFLSTLTIDGVTRSDQGLYTCAASSGLMTK | |
| | KNSTFVRVHEKDKTHTCPPCPAPELLGGPSVFLFPPKPK | |
| | DTLMISRTPEVTCVVVDVSHEDPEVKFNWYVDGVEVHNA | |
| | KTKPREEQYNSTYRVVSVLTVLHQDWLNGKEYKCKVSNK | |
| | ALPAPIEKTISKAKGQPREPQVYTLPPSRDELTKNQVSL | |
| | TCLVKGFYPSDIAVEWESNGQPENNYKTTPPVLDSDGS | |
| | FFLYSKLTVDKSRWQQGNVFSCSVMHEALHNHYTQKSLS | |
| | LSPG | |
| Aflibercept- | SDTGRPFVEMYSEIPEIIHMTEGRELVIPCRVTSPNIT | SEQ ID |
| VEGF | VTLKKFPLDTLIPDGKRIIWDSRKGFIISNATYKEIGLL | NO: 267 |
| binding | TCEATVNGHLYKTNYLTHRQTNTII | |
| domain | | |
| Ranibizumab | DIQLTQSPSSLSASVGDRVTITCSASQDISNYLNWYQQK | SEQ ID |
| Light | PGKAPKVLIYFTSSLHSGVPSRFSGSGSGTDFTLTISSL | NO: 268 |
| Chain | QPEDFATYYCQQYSTVP, VTFGQGTKVEIKRTVAAPSVF | |
| | IFPPSDEQLKSGTASVVCLLNNFYPREAKVQ, VKVDNAL | |
| | QSGNSQESVTEQDSKDSTYSLSSTLTLSKADYEKHKVYA | |
| | CEVTHQGLSSPVTKSFNRGEC | |
| Ranibizumab | EVQLVESGGGLVQPGGSLRLSCAASGYDFTHYMNWVRQA | SEQ ID |
| Heavy | PGKGLEWVGWINTYTGEPTYAADFKRRFTFSLDTSKSTA | NO: 269 |
| Chain | YLQMNSLRAEDTAVYYCAKYPYYYGTSHWYFDVWGQGTL | |
| | VTVSSASTKGPSVFPLAPSSKSTSGGTAALGCLVK | |
| | DYFPEPVTVSWNSGALTSGVHTFPAVLQSSGLYSLSSV | |
| | VTVPSSSLGTQTYICNVNHKPSNTKVDKKVEPKSCDKTH | |
| | L | |

1. A multi-specific antigen binding protein comprising at least two distinct antigen binding regions, wherein a first antigen binding region specifically binds human fibrin or fibrinogen γC domain, and a second antigen binding region specifically binds Vascular Endothelial Growth Factor (VEGF).

2. The multi-specific antigen binding protein of claim 1, wherein the first antigen binding region comprises a heavy chain comprising a variable heavy (VH) chain sequence comprising three heavy chain CDR sequences, CDR-H1, CDR-H2, and CDR-H3, and a light chain comprising a variable light (VL) chain sequence comprising three light chain CDR sequences, CDR-L1, CDR-L2, and CDR-L3, wherein:
a. CDR-H1 comprises the sequence set forth in SEQ ID NOs: 1, 13, 25, 37, 49, 61, 73, 85, 97, 109, 121, 133, 145, 157, 169, 181, 193, 205, 217, 229, or 258;
b. CDR-H2 comprises the sequence set forth in SEQ ID NOs: 2, 14, 26, 38, 50, 62, 74, 86, 98, 110, 122, 134, 146, 158, 170, 182, 194, 206, 218, 230 or 259;
c. CDR-H3 comprises the sequence set forth in SEQ ID NOs: 3, 15, 27, 39, 51, 63, 75, 87, 99, 111, 123, 135, 147, 159, 171, 183, 195, 207, 219, 231, or 260;
d. CDR-L1 comprises the sequence set forth in SEQ ID NOs: 4, 16, 28, 40, 52, 64, 76, 88, 100, 112, 124, 136, 148, 160, 172, 184, 196, 208, 220, 232 or 262;
e. CDR-L2 comprises the sequence set forth in SEQ ID NOs: 5, 17, 29, 41, 53, 65, 77, 89, 101, 113, 125, 137, 149, 161, 173, 185, 197, 209, 221, 233 or 263; and
f. CDR-L3 comprises the sequence set forth in SEQ ID NOs: 6, 18, 30, 42, 54, 66, 78, 90, 102, 114, 126, 138, 150, 162, 174, 186, 198, 210, 222, 234 or 264.

3. The multi-specific antigen binding protein of claim 1, wherein the first antigen binding region comprises a VH sequence selected from a sequence set forth in one of SEQ ID NOs: 7, 19, 31, 43, 55, 67, 79, 91, 103, 115, 127, 139, 151, 163, 175, 187, 199, 211, 223, 235, or 254.

4. The multi-specific antigen binding protein of claim 1 or 2, wherein the first antigen binding region comprises a VL sequence selected from a sequence set forth in SEQ ID NOs: 10, 22, 34, 46, 58, 70, 82, 94, 106, 118, 130, 142, 154, 166, 178, 190, 202, 214, 226, 238 or 255.

5. The multi-specific antigen binding protein of any one of the above claims, wherein the first antigen binding region comprises a VH sequence selected from a sequence set forth in one of SEQ ID Nos: 7, and the VL sequence set for in SEQ ID NO: 10.

6. The multi-specific antigen binding protein of any one of the above claims, wherein the first antigen binding region comprises a VH sequence set forth in SEQ ID NO: 19 and a VL sequence set forth in SEQ ID NO: 22.

7. The multi-specific antigen binding protein of any one of the above claims, wherein the first antigen binding region comprises a VH sequence set forth in SEQ ID NO: 31 and a VL sequence set forth in SEQ ID NO: 34.

8. The multi-specific antigen binding protein of any one of the above claims, wherein the first antigen binding region comprises a VH sequence set forth in SEQ ID NO: 43 and a VL sequence set forth in SEQ ID NO: 46.

9. The multi-specific antigen binding protein of any one of the above claims, wherein the first antigen binding region comprises a VH sequence set forth in SEQ ID NO: 55 and a VL sequence set forth in SEQ ID NO: 58.

10. The multi-specific antigen binding protein of any one of the above claims, wherein the first antigen binding region comprises a VH sequence set forth in SEQ ID NO: 67 and a VL sequence set forth in SEQ ID NO: 70.

11. The multi-specific antigen binding protein of any one of the above claims, wherein the first antigen binding region comprises a VH sequence set forth in SEQ ID NO: 79 and a VL sequence set forth in SEQ ID NO: 82.

12. The multi-specific antigen binding protein of any one of the above claims, wherein the first antigen binding region comprises a VH sequence set forth in SEQ ID NO: 91 and a VL sequence set forth in SEQ ID NO: 94.

13. The multi-specific antigen binding protein of any one of the above claims, wherein the first antigen binding region comprises a VH sequence set forth in SEQ ID NO: 103 and a VL sequence set forth in SEQ ID NO: 106.

14. The multi-specific antigen binding protein of any one of the above claims, wherein the first antigen binding region comprises a VH sequence set forth in SEQ ID NO: 115 and a VL sequence set forth in SEQ ID NO: 118.

15. The multi-specific antigen binding protein of any one of the above claims, wherein the first antigen binding region comprises a VH sequence set forth in SEQ ID NO: 127 and a VL sequence set forth in SEQ ID NO: 130.

16. The multi-specific antigen binding protein of any one of the above claims, wherein the first antigen binding region comprises a VH sequence set forth in SEQ ID NO: 139 and a VL sequence set forth in SEQ ID NO: 142.

17. The multi-specific antigen binding protein of any one of the above claims, wherein the first antigen binding region comprises a VH sequence set forth in SEQ ID NO: 151 and a VL sequence set forth in SEQ ID NO: 154.

18. The multi-specific antigen binding protein of any one of the above claims, wherein the first antigen binding region comprises a VH sequence set forth in SEQ ID NO: 163 and a VL sequence set forth in SEQ ID NO: 166.

19. The multi-specific antigen binding protein of any one of the above claims, wherein the first antigen binding region comprises a VH sequence set forth in SEQ ID NO: 175 and a VL sequence set forth in SEQ ID NO: 178.

20. The multi-specific antigen binding protein of any one of the above claims, wherein the first antigen binding region comprises a VH sequence set forth in SEQ ID NO: 187 and a VL sequence set forth in SEQ ID NO: 190.

21. The multi-specific antigen binding protein of any one of the above claims, wherein the first antigen binding region comprises a VH sequence set forth in SEQ ID NO: 199 and a VL sequence set forth in SEQ ID NO: 202.

22. The multi-specific antigen binding protein of any one of the above claims, wherein the first antigen binding region comprises a VH sequence set forth in SEQ ID NO: 211 and a VL sequence set forth in SEQ ID NO: 214.

23. The multi-specific antigen binding protein of any one of the above claims, wherein the first antigen binding region comprises a VH sequence set forth in SEQ ID NO: 223 and a VL sequence set forth in SEQ ID NO: 226.

24. The multi-specific antigen binding protein of any one of the above claims, wherein the first antigen binding region comprises a VH sequence set forth in SEQ ID NO: 235 and a VL sequence set forth in SEQ ID NO: 238.

25. The multi-specific antigen binding protein of any one of the above claims, wherein the first antigen binding region comprises a VH sequence set forth in SEQ ID NO: 256 and a VL sequence set forth in SEQ ID NO: 257.

26. The multi-specific antigen binding protein of any one of the above claims, wherein the first antigen binding region comprises a humanized, human or chimeric first antigen binding region.

27. The multi-specific antigen binding protein of claim 26, wherein the first antigen binding region comprises a humanized antibody.

28. The multi-specific antigen binding protein of any one of the above claims, wherein the first antigen binding region comprises a heavy chain human Fc region of a class selected from IgG, IgA, IgD, IgE, and IgM.

29. The multi-specific antigen binding protein of claim 28, wherein the human Fc region comprises a human heavy chain constant region of the class IgG and a subclass selected from IgG1, IgG2, IgG3, and IgG4.

30. The multi-specific antigen binding protein of claim 29, wherein the human Fc region comprises wild-type, human IgG1 Fe.

31. The multi-specific antigen binding protein of claim 30, wherein the human Fc domain comprises a sequence set forth in SEQ ID NOs: 8, 20, 32, 44, 56, 68, 80, 92, 104, 116, 128, 140, 152, 164, 176, 188, 200, 212, 224, or 236.

32. The multi-specific antigen binding protein of any one of the above claims, wherein the heavy chain comprises a constant heavy chain sequence set forth by SEQ ID NOs: 8, 20, 32, 44, 56, 68, 80, 92, 104, 116, 128, 140, 152, 164, 176, 188, 200, 212, 224, or 236.

33. The multi-specific antigen binding protein of any one of the above claims, wherein the light chain comprises a constant light chain sequence set forth by SEQ ID NOs: 9, 21, 33, 45, 57, 69, 81, 93, 105, 117, 129, 141, 153, 165, 177, 189, 201, 213, 225, or 237.

34. The multi-specific antigen binding protein of any one of the above claims, wherein the first antigen binding region comprises the VH sequence set forth in SEQ ID NO: 7, and the VL sequence set forth in SEQ ID NO: 10; and the human Fc region comprises wild-type, human IgG1 Fc.

35. The multi-specific antigen binding protein of any one of the above claims, wherein the first antigen binding region comprises the VH sequence set forth in SEQ ID NO: 19, and the VL sequence set forth in SEQ ID NO: 22; and the human Fc region comprises wild-type, human IgG1 Fc.

36. The multi-specific antigen binding protein of any one of the above claims, wherein the first antigen binding region comprises the VH sequence set forth in SEQ ID NO: 31, and the VL sequence set forth in SEQ ID NO: 34; and the human Fc region comprises wild-type, human IgG1 Fc.

37. The multi-specific antigen binding protein of any one of the above claims, wherein the first antigen binding region comprises the VH sequence set forth in SEQ ID NO: 43, and the VL sequence set forth in SEQ ID NO: 46; and the human Fc region comprises wild-type, human IgG1 Fc.

38. The multi-specific antigen binding protein of any one of the above claims, wherein the first antigen binding region comprises the VH sequence set forth in SEQ ID NO: 55, and the VL sequence set forth in SEQ ID NO: 58; and the human Fc region comprises wild-type, human IgG1 Fc.

39. The multi-specific antigen binding protein of any one of the above claims, wherein the first antigen binding region comprises the VH sequence set forth in SEQ ID NO: 67, and the VL sequence set forth in SEQ ID NO: 70; and the human Fc region comprises wild-type, human IgG1 Fc.

40. The multi-specific antigen binding protein of any one of the above claims, wherein the first antigen binding region comprises the VH sequence set forth in SEQ ID NO: 79, and the VL sequence set forth in SEQ ID NO: 82; and the human Fc region comprises wild-type, human IgG1 Fc.

41. The multi-specific antigen binding protein of any one of the above claims, wherein the first antigen binding region comprises the VH sequence set forth in SEQ ID NO: 91, and the VL sequence set forth in SEQ ID NO: 94; and the human Fc region comprises wild-type, human IgG1 Fc.

42. The multi-specific antigen binding protein of any one of the above claims, wherein the first antigen binding region comprises the VH sequence set forth in SEQ ID NO: 103, and the VL sequence set forth in SEQ ID NO: 106; and the human Fc region comprises wild-type, human IgG1 Fc.

43. The multi-specific antigen binding protein of any one of the above claims, wherein the first antigen binding region comprises the VH sequence set forth in SEQ ID NO: 115, and the VL sequence set forth in SEQ ID NO: 118; and the human Fc region comprises wild-type, human IgG1 Fc.

44. The multi-specific antigen binding protein of any one of the above claims, wherein the first antigen binding region comprises the VH sequence set forth in SEQ ID NO: 127, and the VL sequence set forth in SEQ ID NO: 130; and the human Fc region comprises wild-type, human IgG1 Fc.

45. The multi-specific antigen binding protein of any one of the above claims, wherein the first antigen binding region comprises the VH sequence set forth in SEQ ID NO: 139, and the VL sequence set forth in SEQ ID NO: 142; and the human Fc region comprises wild-type, human IgG1 Fc.

46. The multi-specific antigen binding protein of any one of the above claims, wherein the first antigen binding region comprises the VH sequence set forth in SEQ ID NO: 151, and the VL sequence set forth in SEQ ID NO: 154; and the human Fc region comprises wild-type, human IgG1 Fc.

47. The multi-specific antigen binding protein of any one of the above claims, wherein the first antigen binding region comprises the VH sequence set forth in SEQ ID NO: 163, and the VL sequence set forth in SEQ ID NO: 166; and the human Fc region comprises wild-type, human IgG1 Fc.

48. The multi-specific antigen binding protein of any one of the above claims, wherein the first antigen binding region comprises the VH sequence set forth in SEQ ID NO: 175, and the VL sequence set forth in SEQ ID NO: 178; and the human Fc region comprises wild-type, human IgG1 Fc.

49. The multi-specific antigen binding protein of any one of the above claims, wherein the first antigen binding region comprises the VH sequence set forth in SEQ ID NO: 187, and the VL sequence set forth in SEQ ID NO: 190; and the human Fc region comprises wild-type, human IgG1 Fc.

50. The multi-specific antigen binding protein of any one of the above claims, wherein the first antigen binding region comprises the VH sequence set forth in SEQ ID NO: 199, and the VL sequence set forth in SEQ ID NO: 202; and the human Fc region comprises wild-type, human IgG1 Fe.

51. The multi-specific antigen binding protein of any one of the above claims, wherein the first antigen binding region comprises the VH sequence set forth in SEQ ID NO: 211, and the VL sequence set forth in SEQ ID NO: 214; and the human Fc region comprises wild-type, human IgG1 Fc.

52. The multi-specific antigen binding protein of any one of the above claims, wherein the first antigen binding region comprises the VH sequence set forth in SEQ ID NO: 223, and the VL sequence set forth in SEQ ID NO: 226; and the human Fc region comprises wild-type, human IgG1 Fe.

53. The multi-specific antigen binding protein of any one of the above claims, wherein the first antigen binding region comprises the VH sequence set forth in SEQ ID NO: 235, and the VL sequence set forth in SEQ ID NO: 238; and the human Fc region comprises wild-type, human IgG1 Fe.

54. The multi-specific antigen binding protein of any one of claims 28-53, wherein the Fe region comprises one or more amino acid substitutions, wherein the one or more substitutions result in increased half-life, increased ADCC activity, increased ADCP activity, or increased CDC activity compared with the Fe without the one or more substitutions.

55. The multi-specific antigen binding protein of any one of the above claims, wherein the Fe region binds an Fcγ Receptor selected from the group consisting of: FcγRI, FcγRIIa, FcγRIIb, FcγRIIc, FcγRIIIa, and FcγRIIIb.

56. The multi-specific antigen binding protein of any one of the above claims, wherein the first antigen binding region comprises a monoclonal first antigen binding region.

57. The multi-specific antigen binding protein of any one of the above claims, wherein the first antigen binding region binds an γ377-395 epitope of the fibrin or fibrinogen γC domain.

58. The multi-specific antigen binding protein of any one of the above claims, wherein the first antigen binding region binds to peptide comprising an amino acid sequence set forth in at least one of SEQ ID NOs: 241, and 249-253 with a K_{D} of less than or equal to about 1, 2, 3, 4, 5, 6, 7, or 8×10⁻⁵ M, as measured by surface plasmon resonance (SPR) single cycle kinetics (SCK) assay.

59. The multi-specific antigen binding protein of any one of the above claims, wherein the first antigen binding region binds to a peptide comprising the sequence of the γ377-395 epitope of the human fibrin or fibrinogen γC domain with a K_{D} of less than or equal to about 8×10⁻⁵ M, as measured by surface plasmon resonance (SPR) single cycle kinetics (SCK) assay.

60. The multi-specific antigen binding protein of any one of the above claims, wherein the first antigen binding region inhibits Mac-1 binding to fibrin or fibrinogen γC domain.

61. The multi-specific antigen binding protein of any one of the above claims, wherein the first antigen binding region exhibits inhibition of microglial adhesion to the fibrin or fibrinogen γC domain.

62. The multi-specific antigen binding protein of any one of the above claims, wherein the first antigen binding region binds human fibrin at any one of amino acid residues Lys 411, Ile 412, Ile 413, Phe 415, Asn 416, Arg 417, Leu 418, Thr 419, Ile 420, and Gly 421.

63. The multi-specific antigen binding protein of claim 62, wherein the first antigen binding region binds human fibrin at at least two, three, four, five, six, seven, eight, nine, or all ten of amino acid residues Lys 411, Ile 412, Ile 413, Phe 415, Asn 416, Arg 417, Leu 418, Thr 419, Ile 420, and Gly 421.

64. The multi-specific antigen binding protein of any one of the above claims, wherein the first antigen binding region comprises a VH region comprising a paratope that comprises any one of amino acid residues Ser 31, Tyr 32, Trp 33, His 35, Trp 47, Leu 50, Asp 52, Asp 54, Tyr 56, Ala 93, Ser 94, Ser 95, Lys 96 or Asp 96, Pro 97 or Ala 97, Gly 101, Gly 102, and Trp 103.

65. The multi-specific antigen binding protein of claim 64, wherein the first antigen binding region comprises a VH region comprising a paratope that comprises at least two, three, four, five, six, seven, eight, nine, ten, eleven, twelve, thirteen, fourteen, fifteen, sixteen, or all seventeen of amino acid residues Ser 31, Tyr 32, Trp 33, His 35, Trp 47, Leu 50, Asp 52, Asp 54, Tyr 56, Ala 93, Ser 94, Ser 95, Lys 96 or Asp 96, Pro 97 or Ala 97, Gly 101, Gly 102, and Trp 103.

66. The multi-specific antigen binding protein of claim 64, wherein the first antigen binding region comprises a VH region comprising a paratope that comprises amino acid residues Ser 31, Trp 33, His 35, Asp 52, Asp 54, Tyr 56, Ser 94, Gly 101, Gly 102, and Trp 103.

67. The multi-specific antigen binding protein of claim 64, wherein the first antigen binding region comprises a VH region comprising a paratope that comprises amino acid residues Ser 31, Trp 33, His 35, Asp 52, Asp 54, Tyr 56, Ala 93, Ser 94, Lys 96, Pro 97, Gly 101, Gly 102, and Trp 103.

68. The multi-specific antigen binding protein of claim 64, wherein the first antigen binding region comprises a VH region comprising a paratope that comprises amino acid residues Ser 31, Tyr 32, Trp 33, His 35, Trp 47, Asp 52, Asp 54, Tyr 56, Ser 94, Ser 95, Asp 96, Ala 97, Gly 101, Gly 102, and Trp 103.

69. The multi-specific antigen binding protein of any one of claims 62-68, wherein the first antigen binding region comprises a VL region comprising a paratope that comprises any one of amino acid residues His 27, Tyr 32, Tyr 36, Leu 46, Tyr 49, Gln 50, Ala 91 or Asn 91, Leu 92, Leu 94, and Leu 96.

70. The multi-specific antigen binding protein of claim 69, wherein the first antigen binding region comprises a VL region comprising a paratope that comprises at least two, three, four, five, six, seven, eight, nine or all ten amino acid residues His 27, Tyr 32, Tyr 36, Leu 46, Tyr 49, Gln 50, Ala 91 or Asn 91, Leu 92, Leu 94, and Leu 96.

71. The multi-specific antigen binding protein of claim 69, wherein the first antigen binding region comprises a VL region comprising a paratope that comprises the amino acid residues His 27, Tyr 32, Tyr 36, Leu 46, Gln 50, Leu 92, Leu 94, and Leu 96.

72. The multi-specific antigen binding protein of claim 69, wherein the first antigen binding region comprises a VL region comprising a paratope that comprises the amino acid residues His 27, Tyr 32, Tyr 36, Leu 46, Gln 50, Asn 91, Leu 92, Leu 94, and Leu 96.

73. The multi-specific antigen binding protein of claim 69, wherein the first antigen binding region comprises a VL region comprising a paratope that comprises the amino acid residues His 27, Tyr 32, Tyr 36, Leu 46, Tyr 49, Gln 50, Ala 91, Leu 92, Leu 94, and Leu 96.

74. The multi-specific antigen binding protein of any one of the above claims, wherein the first antigen binding region comprises a humanized, human or chimeric antibody.

75. The multi-specific antigen binding protein of claim 74, wherein the first antigen binding region comprises a humanized antibody.

76. The multi-specific antigen binding protein of any one of the above claims, wherein the first antigen binding region comprises a heavy chain human Fc of a class selected from IgG, IgA, IgD, IgE, and IgM.

77. The multi-specific antigen binding protein of any one of claims 28-76, wherein the human Fc region comprises a human heavy chain constant region of the class IgG and a subclass selected from IgG1, IgG2, IgG3, and IgG4.

78. The multi-specific antigen binding protein of claim 77, wherein the human Fc region comprises wild-type, human IgG1 Fc.

79. The multi-specific antigen binding protein of claim 77, wherein the human Fc domain comprises a sequence set forth in SEQ ID NOs: 8, 20, 32, 44, 56, 68, 80, 92, 104, 116, 128, 140, 152, 164, 176, 188, 200, 212, 224, or 236.

80. The multi-specific antigen binding protein of any one of claims 28-79, wherein the Fc region comprises one or more amino acid substitutions, wherein the one or more substitutions results in increased half-life, increased ADCC activity, increased ADCP activity, or increased CDC activity compared with the Fc without the one or more substitutions.

81. The multi-specific antigen binding protein of any one of claims 28-80, wherein the Fe region binds an Fcγ Receptor selected from the group consisting of: FcγRI, FcγRIIa, FcγRIIb, FcγRIIc, FcγRIIIa, and FcγRIIIb.

82. The multi-specific antigen binding protein of any one of the above claims, wherein the first antigen binding region comprises a monoclonal antibody.

83. The multi-specific antigen binding protein of any one of the above claims, wherein the first antigen binding region binds to a peptide comprising an amino acid sequence set forth in at least one of SEQ ID NOs: 241, and 249-253 with a K_{D} of less than or equal to about 1, 2, 3, 4, 5, 6, 7, or 8×10⁻⁵ M, as measured by surface plasmon resonance (SPR) single cycle kinetics (SCK) assay.

84. The multi-specific antigen binding protein of any one of the above claims, wherein the first antigen binding region binds to a peptide comprising the sequence of the γ377-395 epitope of the human fibrin or fibrinogen γC domain with a K_{D} of less than or equal to about 8×10⁻⁵ M, as measured by surface plasmon resonance (SPR) single cycle kinetics (SCK) assay.

85. The multi-specific antigen binding protein of any one of the above claims, wherein the first antigen binding region inhibits Mac-1 binding to fibrin or fibrinogen γC domain.

86. The multi-specific antigen binding protein of any one of the above claims, wherein the first antigen binding region exhibits inhibition of microglial adhesion to the fibrin or fibrinogen γC domain.

87. The multi-specific antigen binding protein of any one of the above claims, wherein the second antigen binding region comprises a heavy chain comprising a second variable heavy (VH) chain sequence comprising three heavy chain CDR sequences, CDR-H1, CDR-H2, and CDR-H3, and a light chain comprising a second variable light (VL) chain sequence comprising three light chain CDR sequences, CDR-L1, CDR-L2, and CDR-L3.

88. The multi-specific antigen binding protein of any one of the above claims, wherein the second antigen binding region binds one or more of VEGF-A, VEGF-B, VEGF-C and VEGF-D.

89. The multi-specific antigen binding protein of any one of the above claims, wherein the second antigen binding region comprises a VH amino acid sequence that is 90% or more, 95% or more, or 99% or more identical to the VH amino acid sequence set forth in SEQ ID NO: 269.

90. The multi-specific antigen binding protein of any one of the above claims, wherein the second antigen binding region comprises a VL amino acid sequence that is 90% or more, 95% or more, or 99% or more identical to the VL amino acid sequence set forth in SEQ ID NO: 268.

91. The multi-specific antigen binding protein of any one of the above claims, wherein the second antigen binding region binds an epitope of VEGF-A comprising at least one of amino acids 82-91 set forth in SEQ ID NO. 265.

92. The multi-specific antigen binding protein of any one claims 1-91, wherein the second antigen binding region comprises a sequence that is 90% or more, 95% or more, or 99% or more identical to the amino acid sequence set forth in SEQ ID NOs: 266 or 267.

93. The multi-specific antigen binding protein of any one of the above claims, wherein the second antigen binding region does not comprise an Fc domain.

94. The multi-specific antigen binding protein of any one of the above claims, comprising a heterodimeric Fc domain.

95. The multi-specific antigen binding protein of any one of the above claims, wherein the multi-specific antigen binding protein comprises a bi-specific antibody.

96. The multi-specific antigen binding protein of any one of the above claims, wherein the multi-specific antigen binding protein consists of a bi-specific antibody.

97. The multi-specific antigen binding protein of any one of the above claims, comprising one or more additional antigen binding domain that specifically binds to human fibrin or fibrinogen γC domain.

98. The multi-specific antigen binding protein of any one of the above claims, comprising one or more additional antigen binding domain that specifically binds to VEGF.

99. The multi-specific antigen binding protein of any one of the above claims, for use in the treatment of a disorder or condition of eye.

100. The multi-specific antigen binding protein of any one of the above claims, formulated for administration to a subject by intravitreal injection.

101. An isolated polynucleotide or set of polynucleotides encoding the first antigen binding region of any of the above claims, a VH thereof, a VL thereof, a light chain thereof, a heavy chain thereof, or an antigen-binding portion thereof; optionally wherein the polynucleotide or set of polynucleotides comprises cDNA.

102. An isolated polynucleotide or set of polynucleotides encoding the second antigen binding region of any of the above claims, a VH thereof, a VL thereof, a light chain thereof, a heavy chain thereof, or an antigen-binding portion thereof; optionally wherein the polynucleotide or set of polynucleotides comprises cDNA.

103. A vector or set of vectors comprising the polynucleotide or set of polynucleotides of claim 101 or 102.

104. A host cell comprising the polynucleotide or set of polynucleotides of claim 101 or 102 or the vector or set of vectors of claim 103.

105. A pharmaceutical composition comprising the multi-specific antigen binding protein of any one of claims 1-100 and a pharmaceutically acceptable excipient.

106. A kit comprising the multi-specific antigen binding protein of any one of claims 1-100 or a pharmaceutical composition of claim 105 and instructions for use

107. A method for treating a disorder or condition of the eye, the method comprising administering to a mammalian subject a therapeutically effective amount the multi-specific antigen binding protein of any one of claims 1-100 or a pharmaceutical composition of claim 105.

108. The method of claim 107, wherein the disorder or condition of the eye is selected from the group consisting of: retinitis pigmentosa, age-related macular degeneration, glaucoma, diabetic retinopathy, uveitis, and retinal detachment.

109. A method for treating a pathology associated with Mac-1 binding to fibrin or Mac-1 binding with fibrinogen, the method comprising administering to a mammalian subject a therapeutically effective amount the multi-specific antigen binding protein of any one of claims 1-100 or a pharmaceutical composition of claim 105.

110. A method of inhibiting microglia activation, the method comprising administering to a mammalian subject a therapeutically effective amount of multi-specific antigen binding protein of any one of claims 1-100 or a pharmaceutical composition of claim 105.

111. A method of producing a multi-specific antigen binding protein, the method comprising expressing a multi-specific antigen binding protein, or antigen binding region thereof in the host cell of claim 104 and isolating the expressed multi-specific antigen binding protein, or antigen binding region thereof.

112. A method of preventing a disorder or condition of the eye, the method comprising administering to a mammalian subject a therapeutically effective amount the multi-specific antigen binding protein of any one of claims 1-100 or the pharmaceutical composition of claim 105.

113. A method of treating a disorder or condition of the eye associated with increased vascularization in a subject in need thereof, comprising administering to the subject the multi-specific antigen binding protein of any one of claims 1-100 or the pharmaceutical composition of claim 105.

114. A method of preventing a disorder or condition of the eye associated with increased vascularization in a subject in need thereof, comprising administering to the subject the multi-specific antigen binding protein of any one of claims 1-100 or the pharmaceutical composition of claim 105.

115. The multi-specific antigen binding protein of any one of claims 1-100, wherein the first antigen binding region comprises the VH sequence set forth in SEQ ID NO: 256, and the VL sequence set forth in SEQ ID NO: 257.
